(19) United States
(12) Patent Application Publication
(10) Pub. No.: US 2026/0091162 A1
(43) Pub. Date: Apr. 2, 2026

(54) SPRAYABLE POLYMERIC SCAFFOLD LOADED WITH INFLAMMASOME-INHIBITING LIPID NANORODS FOR ANTI-INFLAMMATION THERAPY

(71) Applicants: Ashish Kulkarni, Arlington, MA (US); Dhanashree Surve, Miami, FL (US)

(72) Inventors: Ashish Kulkarni, Arlington, MA (US); Dhanashree Surve, Miami, FL (US)

(21) Appl. No.: 19/346,360

(22) Filed: Sep. 30, 2025

Related U.S. Application Data
(60) Provisional application No. 63/701,502, filed on Sep. 30, 2024.

(51) Int. Cl. A61L 27/54; A61L 27/18; A61L 27/22; A61L 27/26; A61L 27/44

(52) U.S. Cl. CPC A61L 27/54; A61L 27/18; A61L 27/227; A61L 27/26; A61L 27/44; A61L 2300/204; A61L 2300/41; A61L 2300/426; A61L 2300/432; A61L 2300/624; A61L 2300/802; A61L 2400/12

(57) ABSTRACT

A sprayable polymeric scaffold composition for localized anti-inflammatory therapy is disclosed. The composition comprises lipid nanorods encapsulating inflammasome inhibitors, a thermogelling agent, and a thickening agent. The lipid nanorods, which can be formed using pyridoxine dipalmitate and DSPE-PEG (2000) carboxylic acid, inhibit NLRP3 and AIM2 inflammasomes, reducing inflammation. The thermogelling agent, such as poloxamer 407, enables the composition to transition from a fluid sol state at room temperature to a semi-solid gel state at body temperature, forming a localized gel film upon topical application. The thickening agent, such as mucin, enhances viscosity and provides additional anti-inflammatory effects. The composition delivers sustained release of encapsulated inhibitors, effectively reducing inflammatory markers and symptoms in conditions such as psoriasis. The sprayable scaffold offers a novel, localized treatment platform with improved skin penetration, ease of administration, and reduced systemic side effects.

### PRIORITY

This application claims the benefit of the filing date of U.S. application No. 63/701,502, filed on Sep. 30, 2024, the disclosures of which is incorporated by reference herein.

### GOVERNMENT SUPPORT

This invention was made with government support under 2142917 awarded by the National Science Foundation. The government has certain rights in the invention.

### FIELD OF THE INVENTION

The present disclosure relates to pharmaceutical compositions for dermatological applications, specifically to sprayable polymeric scaffolds for localized anti-inflammatory therapy.

### BACKGROUND

Psoriasis is a chronic inflammatory disease affecting 125 million people worldwide and was evaluated as high in the global burden of disease study (1). Psoriasis is associated with increased inflammatory arthritis, metabolic disorders, and affects mental health substantially (1,2). Psoriasis involves the development of hyperkeratosis, vascular hyperplasia, and infiltration of immune cells (3,4). The keratinocytes are responsible for the initiation and maintenance phases of psoriasis (5). External stimuli expedite stressed keratinocyte formation and instigate the conversion of plasmacytoid dendritic cells (pDC) to myeloid dendritic cells (mDC) in the presence of inflammatory cytokines, including tumor necrosis growth factor alpha (TNF-α), interleukin-1-beta (IL-1β), and interferons (IFNα/γ) (5). The activated keratinocytes release chemokines, including chemokine (C—X—C motif) ligand 1/2/3 (CXCL1/2/3) and C—C motif chemokine ligand 2 (CCL2), to recruit leukocytes like T cells (adaptive immune cells), dendritic cells, and macrophages to induce innate immunity (5). Also, nucleotide-binding domain, leucine-rich-containing family, pyrin-domain-containing-1 (NLRP1), nucleotide-binding domain, leucine-rich-containing family, pyrin-domain-containing-3 (NLRP3), absent in melanoma 2 (AIM2) inflammasomes, nuclear factor kappa-light-chain-enhancer of activated B cells (NF-κB), and Gasdermin-D have been shown to aggravate psoriasis (6-9). Vitamin D3 derivatives (10), immunosuppressants (11), anti-inflammatory small molecules and biologics (12), steroids (13), retinoids (14), and phototherapy (15) are currently administered to treat psoriasis. However, these treatments suffer from drawbacks, including chronic and acute systemic intoxication, tachyphylaxis, localized irritation, pustulation, skin atropy, striations, telangiectasias, and diminished potency (13,16).

### SUMMARY

Inflammasomes, including nucleotide-binding domain, leucine-rich-containing family, pyrin-domain-containing-3 (NLRP3), and absent in melanoma 2 (AIM2), are responsible for aggravating psoriasis. Existing treatments for psoriasis suffer from drawbacks such as systemic immunosuppression, an inability to penetrate the stratum corneum, inflammatory responses to the delivery vehicle, and limited efficacy and off-target effects due to systemic administration of inflammasome inhibitors. A localized delivery of inflammasome inhibitors in phagocytic macrophages is a revolutionizing psoriasis treatment.

In one embodiment, the disclosure includes a sprayable polymeric scaffold composition for localized anti-inflammatory therapy comprising a plurality of lipid nanorods, each nanorod comprising one or more lipid nanoparticles encapsulating an inhibitor selected from the group consisting of NLRP3 inflammasome inhibitors, AIM2 inflammasome inhibitors, and combinations thereof; a thermogelling agent; and a thickening agent. The composition is in a fluid sol state at room temperature and transitions to a semi-solid gel state at body temperature upon topical spraying on skin.

In another embodiment, the lipid nanoparticles comprise a vitamin B6 compound or derivative such as pyridoxine dipalmitate, pyridoxine hydrochloride, pyridoxal, or pyridoxal phosphate, together with a pegylated phospholipid such as DSPE-PEG (2000) carboxylic acid, PEG-1000, PEG-3400, mPEG-DSPE, DPPE-PEG, or PEGylated cholesterol. The thermogelling agent is selected from methylcellulose, hydroxypropyl methylcellulose, chitosan and derivatives of chitosan, poly(N-isopropylacrylamide) copolymers, poly(ethylene oxide)-poly(propylene oxide)-poly(ethylene oxide) block copolymers, xyloglucan, gelatin-based systems, poly(organophosphazenes), PEG-PLGA-PEG triblock copolymers, alginate-based systems, poloxamer 188, and poloxamer 407. The thickening agent is selected from methylcellulose, hydroxypropyl methylcellulose, carboxymethylcellulose, microcrystalline cellulose, xanthan gum, guar gum, acacia gum, tragacanth gum, carbomers, polyvinylpyrrolidone, polyethylene glycol, pregelatinized starch, modified starch, sodium alginate, propylene glycol alginate, pectin, gelatin, colloidal silicon dioxide, bentonite clay, carrageenan, and mucin. The inflammasome inhibitor may be MCC950, parthenolide, bay 11-7082, glyburide, tranilast, CY-09, OLT1177, VX-765, oridonin, reversetrol, colchicine, auranofin, INF39, Fc11a-2, β-hydroxybutyrate, NLRP3/AIM2-IN-3, BAL-0028, or disulfuram. In some embodiments, the composition is packaged in a spraying applicator.

In a further embodiment, the disclosure includes a method for treating an inflammatory skin disorder in a subject in need thereof, comprising topically administering the sprayable polymeric scaffold composition to an affected area of skin, wherein the composition transitions from a fluid sol to a semi-solid gel upon application, forming a localized gel film that provides sustained release of the encapsulated inhibitor and thereby attenuates inflammation. The inflammatory skin disorder may be atopic dermatitis, psoriasis, contact dermatitis, rosacea, seborrheic dermatitis, acne vulgaris, lupus erythematosus, lichen planus, cellulitis, hidradenitis suppurativa, dermatomyositis, scleroderma, urticaria, vasculitis, or folliculitis.

These and other features will be more clearly understood from the following detailed description taken in conjunction with the accompanying drawings and claims.

### BRIEF DESCRIPTION OF THE DRAWINGS

The drawings illustrate generally, by way of example, but not by way of limitation, various embodiments discussed herein.

FIGS. 1A-1D. Design and engineering of sprayable polymeric scaffold loaded with non-spherical pathogen-like trojan-horse lipid nanoparticles. (a) Schematic for the synthesis of non-spherical pathogen-like trojan-horse lipid nanorods. The nanorods, nanoellipses, and nanospheres were prepared using 10:1, 5:1, and 2:1 molar ratios of pyridoxine dipalmitate and DSPE PEG 2000 (carboxylic acid). (b) Mechanism of non-spherical lipid nanoparticle cellular internalization and NLRP3 inflammasome inhibition. The non-spherical lipid nanoellipses/rods were internalized by macropinocytosis and clathrin-mediated endocytosis, while the nanospheres were internalized by phagocytosis. The nanorods delayed lysosomal rupture and significantly reduced the IL-1βlevel by attenuating ASC speck formation, restraining calcium influx, and NLRP3 inflammasome, whereas the nanoellipses and nanospheres reduced the mitochondrial ROS formation. (c) Scaffold design and synthesis. The NLRP3/AIM2-IN-3 (NA3) was loaded within the nanorods, and later, Poloxamer 407 (10% w/v; thermogelling agent) and mucin (2.5 mg/mL; thickening agent) were dissolved in the NA3 nanorods aqueous dispersion to form a solution for topical spraying. When sprayed, the poloxamer solution forms micelles and depicts temperature-dependent gelling properties. (d) In vivo effect of sprayable polymeric scaffold loaded with nanorods or NA3 nanorods on psoriasis. The NA3 nanorod polymeric scaffold, when sprayed, causes a thin film due to gelation. The NA3 nanorods and 8 mucins reduce inflammation, psoriatic chemokines, and keratinocyte proliferation due to their effect on NLRP3 and AIM2 inflammasome inhibition.

FIGS. 2A-2F. Evaluation of lipid nanoparticle shape on internalization and inflammasome inhibition in macrophages. (a) Synthesis and physicochemical characterization of non-spherical pathogen-like Trojan-horse lipid nanoparticles including Transmission electron microscopy, Particle size, and zeta potential distribution, and stability studies at 4° C. (b,c) Mechanism of internalization of nano-spheres/ellipses/rods using flow cytometer ((Mean+SD; n=3 data analyzed by two-way ANOVA and Tukey's multiple comparison test with * p<0.05, ** p<0.01, *** p<0.001, **** p<0.0001). (d,c) Internalization of nano-spheres/ellipses/rods through CD14 and TLR-4 determined by flow cytometry and RT-PCR, respectively (Mean+SD; n=6 and Mean+SEM; n>10 data analyzed by ordinary one-way ANOVA Tukey's multiple comparisons and Brown Forsythe Welche Dunnet's T3 multiple comparisons, respectively with *p<0.05, ** p<0.01, *** p<0.001, **** p<0.0001). (f) Effect of nano-spheres/ellipses/rods on NLRP3 inflammasomes in iBMDMs determined by IL-1βELISA. (Mean+SD; n=6 data analyzed by ordinary two-way ANOVA and Tukey's multiple comparison test with * p<0.05, ** p<0.01, *** p<0.001, **** p<0.0001).

FIGS. 3A-3I. Mechanistic evaluation of lipid nanoparticles (LNPs) shape on inflammasome inhibition in macrophages. (a) Schematic representation of the effect of LNP shapes on ASC speck formation. (b) Confocal microscopic images of ASC speck (CFP) in iBMDM with nano-16 spheres/ellipses/rods at 20λ. (c) ASC speck number per cell when treated with nano-spheres/ellipses/rods. (Mean+SD, n>10 using Brown Forsythe Welche ANOVA and Dunnett's T3 multiple comparison with * p<0.05, ** p<0.01). (d) Schematic representation of the effect of LNP shapes on lysosomal rupture. (c) Confocal microscopic images of intact lysosomes (TRITC) in iBMDM treated with nano-spheres/ellipses/rods at 20×. (f,g) Analysis of intact lysosomes normalized against LNP internalization and live cells as indicated by Cy5 and DAPI signal, respectively when treated with nano-spheres/ellipses/rods. Each data represented as mean±SD; n=7; data analyzed by ordinary one-way ANOVA and Tukey's multiple comparisons with * p<0.05, ** p<0.01, *** p<0.001, * p<0.0001). (h, i) Effect of nano-spheres/ellipses/rods on calcium influx and mitochondrial ROS formation determined by flow cytometer. (Each data represented as mean±SD; n=5 and n=10); data analyzed by Brown Forsythe Welche ANOVA with Dunnets T3 multiple comparison and ordinary one-way ANOVA, respectively with * p<0.05, ** p<0.01, *** p<0.001).

FIGS. 4A-4G. Effect of NLRP3-AIM2 inhibitor (NA3) loaded nanorods on inflammasome inhibition. (a) Schematic of NA3 nanorods synthesis. (b, c) Particle size and zeta potential distribution of NA3 loaded nanorods. (d) Time-dependent stability studies of NA3 nanorods at 4° C. (c) Drug loading and encapsulation efficiency of NA3 in nanorods determined by RP-HPLC. (f) Effect of NA3 loaded nanborods on inflammasome inhibition in iBMDMs determined by IL-1βELISA (Each data represented as mean±SEM; n=5 data analyzed by two-way ANOVA with * p<0.05, ** p<0.01, *** p<0.001, **** p<0.0001). (g) Effect of NA3 nanorods on ASC speck formation determined by confocal microscopy (Each data represented as mean±SEM; n=5 data analyzed by Brown Forsythe and Welche Test and Dunnett's multiple comparison test with * p<0.05, ** p<0.01, *** p<0.001, **** p<0.0001).

FIGS. 5A-5I. Effect of NLRP3-AIM-2 inhibitor (NA3) loaded nanorods polymeric scaffold on inflammasome inhibition. (a) Screening of thermogelling polymer concentration (Data represents mean±SD, n=3 analyzed by one-way ANOVA and Tukey's multiple comparison test with * p<0.05, *** p<0.001, **** p<0.0001). (b,c) Particle size and zeta potential distribution, respectively of NA3 loaded nanorods polymeric scaffold. (d) Time-dependent stability studies of NA3 nanorods polymeric scaffold at 4° C. (e) In-vitro drug release of NA3 from NA3-loaded nanorods polymeric scaffold in PBS and cell lysate. (Each data represents mean±SD; n=4 and analyzed by two-way ANOVA with * p<0.05, ** p<0.01). (f) Effect of NA3 loaded nanorods polymeric scaffold on inflammasome inhibition in iBMDM determined by IL-1βELISA. (Each data represents mean±SD; n=5 and analyzed by two-way ANOVA and Tukey's multiple 22 comparisons with ** p<0.01, **** p<0.0001). (g) Effect of NA3 loaded nanorods and blank nanorods polymeric scaffold on caspase-1 determined by western blot in iBMDM (1-untreated, 2-LPS (Negative control), 3-LPS+Nigericin (positive control), 4-Blank nanorods polymeric scaffold, 5-NA3 nanorods polymeric scaffold treated iBMDM) (Each data represents mean±SD; n=3 and analyzed by one-way ANOVA and Tukey's multiple comparisons with *** p<0.00). (h) Effect of NA3 nanorods polymeric scaffold on ASC speck formation determined by confocal microscopy. (Each data represented as mean±SD; n=5 and analyzed by Brown Forsythe Welche Dunnett's T3 multiple comparison with **** p<0.0001). (i) Rheological studies of NA3 nanorods polymeric scaffold.

FIGS. 6A-6G. In vivo evaluation in Imiquimod (IMQ) induced Psoriasis Balb/C mice model. (a) Study paradigm schematic representation. (b) Cumulative daily PASI score of back scaling, crythema, and thickness (Data represented as mean±SEM, n=4 and analyzed by ANOVA and Tukey's multiple comparisons with ** p<0.01 between NA3 nanorods and blank nanorods scaffold). (c) Daily body weight measurement of IMQ-induced psoriasis mice in each group. (d, c) Back thickness and spleen to body weight ratio of IMQ-induced mice from each group (Each data represented as mean±SD; n=4 data analyzed by two-way ANOVA and Tukey's multiple comparisons with * p<0.05, **** p<0.0001). (f) RNA expression of different cellular 29 components involved in NLRP3 and AIM-2 inflammasome activation extracted from the back skin of Balb/c mice in each group. (Each data represented for n=4 data analyzed by Brown Forsythe and Welche one-way ANOVA Test with * p<0.05, ** p<0.01, *** p<0.001, **** p<0.0001). (g) Western blot analysis of IL-17A expressed in the mice back skin from each group (Each data represented as mean±SD; n=4, data analyzed by one-way ANOVA and Tukey's multiple comparison test with * p<0.05, ** p<0.01).

FIGS. 7A-7D. Microscopic evaluation of in vivo skin samples from Imiquimod (IMQ) induced Psoriasis Balb/C mice model. (a) Light microscopic images of H&E, Ki67 (DAB), and CD31 antibody (DAB) stained preserved back skin transverse sections from euthanized mice on day 6 (Images represent Munro microabscess (black arrow), hyperplasia/hypogranulosis (blue arrow), Capillary proliferation (green arrow), Rete Ridges (orange arrow), Inflammatory infiltration (red arrow). (b) The integrated density of Ki67 DAB stain indicating cell proliferation measured from IHC sections imaged at 20×(n=4) (Data represented as mean±SD; and analyzed by ordinary one-way ANOVA with * p<0.05, ** p<0.01). (c) Mean grey value of CD31 marker indicating vascular hyperproliferation measured for IHC microscopic images at 20×and 40×(Data represented as mean±S.D; n=4 and analyzed by ordinary one-way ANOVA Tukey's multiple comparison with ** p<0.01, *** p<0.001). (d) The inflammatory cell infiltrate 31 count indicating cell proliferation for H&E images at 40× (n=4) (Data represented as mean±SD; and analyzed by Brown Forsythe Welche Dunnet's T3 multiple comparison with * p<0.05, ** p<0.01, *** p<0.001).

FIGS. 8A-8D. In vitro characterization of Trojan-horse LNP. (a) Evaluation of FITC CD14 antibody binding to the CD14 cell surface receptor (Mean+SD, n=6 with 3 biological replicates and 2 technical replicates statistically analyzed by one-way ANOVA). (b) Cytotoxicity studies by MTT assay of nano-spheres/ellipses/rods at different concentrations in iBMDM (data represents mean±SD, n=8 with 3 biological replicate and 3,3, and 2 technical replicate per biological replicate, statistically analyzed by two-way ANOVA and Dunnet's multiple comparisons) (c) Effect of nano-spheres/ellipses/rods on NLRP3 inflammasomes in iBMDM determined by IL-1βELISA when iBMDM were treated with Trojan-horse LNP post-LPS treatment. (data represents mean±SD, n=6 with 3 biological replicate and two technical replicate per biological sample statistically analyzed by one-way ANOVA) (d) Confocal Microscopic images of live and dead iBMDM stained with NucBlue (DAPI) and Propidium iodide (TRITC), respectively after treatment with nano-spheres/ellipse/rods, LPS, and Nigericin to quantify ASC specks (CFP) at 20× magnification.

FIGS. 9A-9C. Effect of nanoparticle shape on the NLRP3 inflammasomes. (a) Microscopic images of iBMDM stained for the nucleus and lysosome with NucBlue (DAPI) and Lysotracker red (TRITC), respectively after treatment with DiD (Cy5) loaded nano-spheres/ellipse/rods, LPS and Nigericin to quantify lysosomes at 20× magnification. (b) Overlay depicting the shift in Calcium influx measured by Fluo-4AM post-treatment of iBMDM with nanospheres/ellipse/rods (c) Overlay depicting the shift in mitochondrial ROS measured by MitoSoX Red post-treatment of iBMDM with nano-spheres/ellipse/rods, LPS, and Nigericin.

FIGS. 10A-10C. Viscosity, chromatogram, and microscopy data detailing the biophysical characteristics of the nanorod-polymeric scaffold composition. (a) Effect of different temperatures on viscosity of thermogelling agent at 10% w/v and 15% w/v, respectively. (b) RP-HPLC chromatogram of NA3 (1000 μg/mL). (c) Microscopic ASC speck images of iBMDM treated with NA3/blank nanorods and their scaffolds which were observed as 60×.

FIGS. 11A-11F. Use of the nanorod-polymeric scaffold composition to treat a mouse model of psoriasis. (a) Representative images of mice treated with Imiquimod (5% w/w; 62.5 mg; Positive control), Vaseline (62.5 mg; Negative control), blank nanorods scaffold, and NA3 nanorods scaffold, respectively on day 0 (before IMQ treatment), day 5 (last day of treatment), and day 6 (before mice were euthanized). (b) IL-1βELISA was analyzed in plasma samples obtained from individual animals on day 6 post euthanasia (n=4 for all the groups and 3 for negative control). (c-e) The average PASI score for scaling (p=0.0059 and 0.0011 on day 4 and 5, respectively for blank nanorods treated group compared with NA3 nanorods scaffold treated group), erythema (p=0.0059 on day 4 for blank nanorods treated group compared with NA3 nanorods scaffold treated group), and back thickness, respectively for individual groups from days 0-6 (n=4 for all the groups and 3 for negative control, data was statistically analyzed by one-way ANOVA and Tukey's multiple comparison test). (f) Representative H&E images (40×) of the back skin transverse section from each group depicting inflammatory cell infiltrate encircled in red.

FIGS. 12A-12C. Representative gating strategy on untreated control group which was applied onto treatment groups. (a) Cellular internalization of trojan-horse nanoparticles in presence of various chemical inhibitors. (b) Effect of nanospheres/ellipses/rods on Calcium influx. (c) Effect of nanospheres/ellipses/rods on Mitochondrial ROS generation.

FIGS. 13A-13F. Effects of nanostructures on mitochondrial ROS formation. (a-d) Effect of nano-spheres/ellipses/rods on mitochondrial ROS formation for four different experiments determined by flow cytometer. (Each data represented as mean±SD; n=2,2,3,3; data analyzed by ordinary one-way ANOVA). (c) Schematic representing the mechanism of cellular internalization of Trojan-horse LNP and its effect on different cellular assemblies impacting NLRP3 inflammasome inhibition. (f) Representative Western Blot images for Caspase-1, IL-17A and β-actin. The separation between pro-caspase-1 (top) and active-caspase-1 (bottom) (n=3 technical replicates). The very high active-caspase-1 expression in the nigericin positive control treatment group is shown to confirm the identity of the lower band as active-caspase-1. Left to right-Untreated, LPS, LPS+Nigericin, Nanorods, NA3. Representative Western blot images of IL-17A (n=4, samples of 2 animals/sample were pooled and two different samples were run for western blot analysis) and β-actin run on mice skin samples treated with different control groups specified as Left to right-IMQ treated (positive control) 1, IMQ treated (positive control) 2, Blank nanorods 1, Blank nanorods 2, NA3 nanorods 1, NA3 nanorods 2, negative control 1, negative control 2.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Psoriasis is a chronic inflammatory skin disorder characterized by keratinocyte hyperproliferation, vascular hyperplasia, and immune cell infiltration, which collectively lead to erythema, scaling, and thickened skin. Current therapeutic approaches, including vitamin D3 derivatives, corticosteroids, retinoids, immunosuppressants, biologics, and phototherapy, have demonstrated limited efficacy and are often associated with significant drawbacks. These include systemic immunosuppression, localized irritation, skin atrophy, tachyphylaxis, and diminished potency over time. Furthermore, existing treatments primarily target keratinocytes and T-cells, overlooking the role of macrophages in perpetuating psoriatic inflammation. Macrophages, particularly tissue-resident macrophages, contribute to the persistence of inflammation through inflammasome activation, releasing cytokines such as IL-1β, which exacerbate keratinocyte proliferation and immune cell recruitment. Despite their importance, macrophage-targeted therapies remain underexplored, and conventional inflammasome inhibitors often lack specificity, potency, and the ability to penetrate the hydrophobic stratum corneum, further limiting their therapeutic potential.

The present disclosure addresses these limitations by introducing a sprayable polymeric scaffold composition loaded with lipid nanorods encapsulating dual inflammasome inhibitors targeting NLRP3 (UniProt accession Q96P20 provides the human NLRP3 amino acid sequence and is incorporated herein by reference) and AIM2 (“Absent in Melanoma; UniProt accession O14862 provides the human AIM2 amino acid sequence and is incorporated herein by reference). The described approach leverages non-spherical lipid nanoparticles, specifically nanorods, which mimic pathogen-like shapes to enhance macrophage adherence and internalization via macropinocytosis and clathrin-mediated endocytosis. This targeted delivery mechanism promotes efficient inhibition of inflammasome activation within macrophages, converting pro-inflammatory phenotypes to anti-inflammatory states. The nanorods are formulated using, for example, pyridoxine dipalmitate, a known inflammasome inhibitor, and DSPE-PEG (2000) carboxylic acid, which stabilizes the nanoparticles and facilitates their self-assembly. To address the challenges of topical delivery, the nanorods are incorporated into a thermogelling polymeric scaffold containing, for example poloxamer 407 and mucin. This scaffold transitions from a fluid sol state at room temperature to a semi-solid gel state at body temperature upon application, forming a localized gel film that provides sustained release of the encapsulated inhibitors. The inclusion of mucin further enhances anti-inflammatory effects and improves skin penetration.

By simultaneously targeting NLRP3 and AIM2 inflammasomes, the described approach achieves synergistic inhibition of inflammatory pathways, reducing significant markers such as IL-1β, caspase-1, CXCL2, and IL-17A, while avoiding systemic side effects. The sprayable scaffold offers a novel, localized treatment platform that addresses the unmet need for safe, effective, and macrophage-targeted anti-psoriatic, and other inflammatory conditions, therapy, paving the way for improved patient outcomes and broader applications in inflammatory dermatological disorders.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R.J. Lewis, John Wiley & Sons, New York, N. Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

A “compound,” as used herein, refers to any type of substance or agent that is commonly considered a drug, or a candidate for use as a drug, as well as combinations and mixtures of the above.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, an “instructional material” includes a publication, a recording, a diagram, or any other medium of expression which can be used to communicate the usefulness of the peptide/protein of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

The term “regulate” refers to either stimulating or inhibiting a function or activity of interest.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this invention.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

### Inflammatory Skin Disorders

An inflammatory skin disorder is a condition characterized by inflammation of the skin, which involves redness, swelling, itching, and sometimes pain or irritation. This inflammation results from an immune system response to various triggers such as infections, allergens, or autoimmune processes.

Examples of inflammatory skin disorders include, but are not limited to, atopic dermatitis (eczema), psoriasis (including plaque psoriasis, guttate psoriasis, pustular psoriasis), contact dermatitis, allergic urticaria (hives), rosacea, seborrheic dermatitis, acne vulgaris, lupus erythematosus (cutaneous lupus), lichen planus, dermatitis herpetiformis (gluten allergy-related), cellulitis, hidradenitis suppurativa, dermatomyositis, scleroderma, urticaria (hives), vasculitis, pyoderma gangrenosum or folliculitis.

Provided herein are novel compositions and methods to treat inflammatory skin disorders.

### Nanorods/Nanoparticles

In embodiments provided herein, the compositions comprise nanorods comprising one or more lipid nanoparticles (LNPs; ranging from about 10 to 1000 nanometers in diameter, composed primarily of lipids), including, for example a pegylated phospholipid, including, but not limited to, phospholipids, cholesterol, ionizable cationic lipids, and polyethylene glycol (PEG)-derived lipids (PEGylated lipids), DSPE PEG (2000) (carboxylic acid), PEG 1000, PEG 3400, mPEG-DSPE, DPPE PEG, PEGylated cholesterol or combination thereof.

In embodiments provided herein, the compositions comprise nanoparticle that comprises vitamin B6 or a derivative thereof, including, but not limited to, pyridoxine dipalmitate, pyridoxine hydrochloride, pyridoxal. pyridoxal phosphate or combination thereof.

### Inhibitors

An inflammasome is a cytosolic multiprotein complex that plays a role in the innate immune system by detecting pathogenic microorganisms and cellular stress signals. It acts as a central regulator of inflammation by assembling in response to specific triggers such as pathogen-associated molecular patterns (PAMPs) or damage-associated molecular patterns (DAMPs). Upon activation, inflammasomes promote the activation of caspase-1, which in turn cleaves and activates pro-inflammatory cytokines like interleukin-1ß (IL-1β) and interleukin-18 (IL-18), leading to an inflammatory response.

Inflammasomes are composed of a sensor protein (e.g., NOD-like receptors such as NLRP3), an adaptor protein (ASC), and the effector enzyme pro-caspase-1. Activation of the inflammasome can also induce a form of inflammatory cell death called pyroptosis, which helps to limit pathogen replication and promote immune defense.

Inflammasome inhibitors are substances or molecules that block or reduce the activation of inflammasomes. By inhibiting inflammasome activation, these inhibitors help to prevent the release of pro-inflammatory cytokines such as IL-1βand IL-18 and reduce inflammatory responses that can contribute to various diseases. Provide herein is a combination NLRP3 inflammasome inhibitors, AIM2 inflammasome inhibitors.

NLRP3 inflammasome inhibitors are compounds or molecules that specifically block or reduce the activation of the NLRP3 inflammasome, a multiprotein complex that plays a key role in the innate immune system by detecting danger signals and triggering inflammation through activation of caspase-1 and subsequent release of pro-inflammatory cytokines IL-1βand IL-18. Inhibiting NLRP3 prevents the excessive inflammatory response associated with many inflammatory and autoimmune diseases.

Examples of NLRP3 inflammasome inhibitors include, but are not limited to, INF39 (a reversible inhibitor of the NLRP3 inflammasome), MCC950 (small-molecule inhibitor that selectively blocks NLRP3 inflammasome activation), CY-09 (Inhibits the ATPase activity of NLRP3), oridonin, parthenolide and VXA-765.

AIM2 inflammasome inhibitors are substances or molecules that block the activation of the AIM2 inflammasome, a cytosolic sensor primarily responsible for detecting double-stranded DNA (dsDNA) in the cytoplasm. The AIM2 inflammasome plays a role in the immune response by assembling upon binding cytosolic dsDNA, which leads to activation of caspase-1 and the release of pro-inflammatory cytokines IL-1βand IL-18, as well as pyroptotic cell death.

Examples of AIM2 inflammasome inhibitors include, but are not limited to, 4-sulfonic calixarenes (compounds that block dsDNA binding to AIM2 and prevent its activation by trapping AIM2), PYD-only proteins (POPs) (decoy proteins that inhibit inflammasome assembly by occupying the pyrin domain (PYD) of AIM2 and its adaptor ASC, blocking necessary protein-protein interactions for inflammasome formation), and small molecule inhibitors.

In some embodiment, the NLRP3 inflammasome inhibitors and/or AIM2 inflammasome inhibitors comprise MCC950 (CRID3; IUPAC name: 1-(1,2,3,5,6,7-hexahydro-s-indacen-4-yl)-3-[4-(2-hydroxypropan-2-yl) furan-2-yl]sulfonylurea; SMILES: COC1=CC═C (C═C1) CC(═O) NC2═CC(═CC═C2) O (for related functional groups), Molecular formula: C20H23N205S.Na; selectively bind and inhibit the NACHT domain of NLRP3, blocking its activation and inflammatory signaling), parthenolide ((1S,2S.4R.7E. 11S)-4,8-dimethyl-12-methylidene-3,14-dioxatricyclo [9.3.0.0²,⁴] tetradec-7-en-13-one), bay 11-7082 (IUPAC name is (2E)-3-[(4-methylphenyl) sulfonyl]prop-2-enenitrile. Its SMILES representation is: C1=CC═C (C═C1) S(═O) (═O) C═CC #N), glyburide (1-[[p-[2-(5-chloro-o-anisamido)-ethyl]phenyl]-sulfonyl]-3-cyclohexylurea), tranilast (N-(3,4-Dimethoxycinnamoyl) anthranilic acid), CY-09 (4-[[4-oxo-2-thioxo-3-[[3-(trifluoromethyl)phenyl]methyl]-5-thiazolidinylidene] methyl]benzoic acid), OLT1177 (dapansutrile; 3-(methylsulfonyl) propanenitrile.), VX-765 (belnacasan; (2S)-1-[(2S)-2-[(4-amino-3-chlorobenzoyl)amino]-3,3-dimethylbutanoyl]-N-[(2R.3S)-2-ethoxy-5-oxooxolan-3-yl] pyrrolidine-2-carboxamide), oridonin ((1S,2S.5S,8R,9S,10S,11R,15S,18R)-9,10,15,18-tetrahydroxy-12,12-dimethyl-6-methylidene-17-oxapentacyclo [7.6.2.1\hat{ }5,8.0\hat{ }1, 11.0\hat{ }2,8] octadecan-7-one.), reversetrol (5-[(E)-2-(4-hydroxyphenyl) ethenyl]benzene-1.3-diol), colchicine (N-[(7S)-1,2,3,10-tetramethoxy-9-oxo-6,7-dihydro-5H-benzo[a] heptalen-7-yl]acetamide), auranofin ([(2R,3R,4S,5R,6S)-3,4,5-tris(acetyloxy)-6-{[(triethyl-lambda5-phosphanylidene) aurio] sulfanyl} oxan-2-yl]methyl acetate), INF39 (ethyl 2-(2-chlorobenzyl) acrylat), Fc11a-2, B-Hydroxybutyrate (BHB) (1-ethyl-5-methyl-2-phenyl-1H-benzo[d]imidazole, 1-ethyl-5-methyl-2-phenyl-1H-benzo[d]imidazole), NLRP3/AIM2-IN-3 (NA3; N-(3-hydroxyphenyl)-2-(1H-indol-6-yl) acetamide), NLRP3 Inflammasome Inhibitor I (also known as MCC950), BAL-0028 (Wilhelmsen et al. J Exp Med. 2025 Sep. 2;222 (11): e20242403. doi: 10.1084/jem.20242403), or disulfuram (N,N-diethylcarbamodithioic diamide (also called N,N-diethyl [(diethylcarbamothioyl)disulfanyl]carbothioamide), NT-0796 (IUPAC name: propan-2-yl (2R)-2-(1,2,3,5,6,7-hexahydro-s-indacen-4-ylcarbamoyloxy)-3-pyrimidin-2-ylpropanoate); IC100 (a humanized monoclonal IgG4 antibody that specifically targets and inhibits the adaptor protein ASC (apoptosis-associated speck-like protein containing a caspase recruitment domain), which is a component in multiple types of inflammasomes (Desu et al. J Neuroinflammation. 2020 May 4; 17:143. doi: 10.1186/s12974-020-01826-0)); obovatol (5-(2-Propenyl)-3-[4-(2-propenyl) phenoxy]-1.2-benzenediol; IUPAC name: 5-(prop-2-en-1-yl)-3-[4-(prop-2-en-1-yl) phenoxy] benzene-1,2-diol) or a combination thereof.

### Topical Compositions

Topical compositions may include topical treatments for wounds and dermatological conditions and can be categorized into several types including, but not limited to: Ointments and creams (e.g., Antibiotic ointments (e.g., bacitracin, neomycin), Corticosteroid creams and/or Moisturizing creams); Gels (e.g., Hydrogels and/or Silicone gels); Foams (e.g., Antimicrobial foams and/or Steroid foams); Sprays (e.g., Antiseptic sprays, Analgesic sprays and/or Treatment application/sprays); Powders (e.g., Antifungal powders and/or Absorbent powders), Patches and dressings (e.g., Hydrocolloid dressings and/or Medicated patches); Solutions (e.g., Antiseptic solutions and/or Medicated solutions); Lotions (e.g., Emollient lotions and/or Medicated lotions); Pastes (e.g., Zinc oxide pastes and/or Calamine lotion); Oils (e.g., Essential oils and/or Mineral oils)′

The topical treatments vary in their formulations, active ingredients, and specific applications, allowing for targeted therapy of various wounds and dermatological conditions.

The use of “applicators and devices for spraying” refers to pump and aerosol spraying applicators comprising of several components, including, but not limited to, Pump Sprayers (e.g., container (holds the liquid formulation, dip tube: extends into the container to draw up the liquid, pump mechanism (e.g., spring-loaded piston or diaphragm; creates pressure to expel the liquid), actuator (button or lever that activates the pump), nozzle (shapes the liquid into a spray pattern), and/or check valves (control liquid flow direction)) or Aerosol Sprayers (pressurized canister (contains the formulation and propellant), propellant (liquefied gas that creates pressure (e.g., hydrofluoroalkanes)), dip tube (extends into the canister to draw up the mixture), metering valve (controls the amount of spray released), actuator (button that opens the valve), spray nozzle (shapes the aerosol into a specific spray pattern) and/or vapor tap (helps mix the propellant and formulation)). Both types may include additional features like: overcaps: (protect the nozzle and prevent accidental discharge, safety mechanisms (prevent unintended activation) and/or adjustable nozzles (allow for different spray patterns).

The topical application can comprise thermogelling, thickening agents or a combination thereof.

### Thermogelling Agents

The “thermogelling agent” in a pharmaceutical composition works by undergoing a temperature-dependent sol-gel transition. At lower temperatures, the composition exists as a free-flowing liquid (sol state). As the temperature increases to body temperature (typically around 37° C.), the thermogelling agent causes the composition to transform into a semi-solid gel state.

This transition occurs due to the unique molecular structure of thermogelling agents, which often consist of amphiphilic block copolymers. These polymers have both hydrophilic and hydrophobic segments. At lower temperatures, the polymers are fully hydrated and exist as individual molecules or small micelles in solution. As the temperature rises, the hydrophobic segments become more dominant, causing the polymers to aggregate and form a three-dimensional network structure, resulting in gelation.

The gel formation provides several benefits in pharmaceutical applications, including, but not limited to, controlled drug release (the gel matrix can entrap drug molecules, allowing for sustained and controlled release over time), increased residence time (the gel adheres to the application site, prolonging the drug's contact with the target tissue, case of administration (the composition can be easily applied as a liquid and then forms a gel in situ, improving patient compliance and case of use) and/or localized drug delivery (the gel formation helps confine the drug to a specific area, reducing systemic exposure and potential side effects).

Common examples of thermogelling agents used in pharmaceutical compositions include poloxamers (e.g., Poloxamer 407), certain cellulose derivatives, and chitosan-based systems. Other examples of thermogelling agents include, but are not limited to, methylcellulose, hydroxypropyl methylcellulose (HPMC), chitosan and its derivatives, poly(N-isopropylacrylamide) (PNIPAM) and its copolymers, poly(ethylene oxide)-(propylene oxide)-poly(ethylene oxide) (PEO-PPO-PEO) block copolymers, xyloglucan, gelatin-based systems, poly(organophosphazenes), poly(ethylene glycol)-poly(lactic acid-co-glycolic acid)-poly(ethylene glycol) (PEG-PLGA-PEG) triblock copolymers, alginate-based systems, poloxamer 188, or poloxamer 407.

### Thickening Agents

The “thickening agent” in a pharmaceutical composition works by increasing the viscosity of the formulation. When added to a liquid or semi-liquid preparation, the thickening agent interacts with the solvent (usually water) to form a network structure that restricts the flow of the liquid, resulting in a more viscous or gel-like consistency. The mechanism of action for thickening agents typically involves one or more of the following processes: hydration (many thickening agents, such as cellulose derivatives and natural gums, absorb water and swell, increasing the overall volume and viscosity of the composition), polymer entanglement (long-chain polymers, like high molecular weight cellulose derivatives, can form physical entanglements, creating a three-dimensional network that increases viscosity), hydrogen bonding (some thickeners form hydrogen bonds with water molecules and other components in the formulation, leading to increased viscosity) and/or particle-particle interactions (colloidal thickeners, such as silica, can form a network of particles that increases the resistance to flow).

The increased viscosity provided by thickening agents offers several benefits in pharmaceutical compositions, including, but not limited to, improved stability (thickeners can prevent sedimentation or separation of suspended particles or emulsions), enhanced drug delivery (the increased viscosity can prolong contact time with the target tissue, potentially improving drug absorption), better spreadability (for topical formulations, thickeners can improve the case of application and adherence to the skin), controlled release (the thickened matrix can modulate the release rate of active ingredients) and/or improved palatability (in oral liquid formulations, thickeners can mask unpleasant tastes and improve mouthfeel).

The choice of thickening agent depends on the specific requirements of the pharmaceutical composition, including the desired viscosity, pH stability, compatibility with other ingredients, and the intended route of administration.

Examples of thickening agents include, but are not limited to, methylcellulose, hydroxypropyl methylcellulose (HPMC), carboxymethylcellulose (CMC), microcrystalline cellulose, xanthan gum, guar gum, acacia gum (gum arabic), tragacanth gum, carbomers, polyvinylpyrrolidone (PVP), polyethylene glycol (PEG), pregelatinized starch, modified starch, sodium alginate, propylene glycol alginate, pectin, gelatin, colloidal silicon dioxide, bentonite clay, carrageenan, or mucin.

### EXAMPLE

The disclosure can be better understood by reference to the following example which is offered by way of illustration. The disclosure is not limited to the example given herein.

### Example 1

### Introduction

The inflammasome is a cluster of cytosolic proteins that are activated due to the oligomerized pathogen recognition receptors (PRR), including the nucleotide-binding domain and leucine-rich-repeat containing family (NLR) or absent in melanoma-2 (AIM2) like receptor (ALR) when encountered with the damage-associated molecular patterns (DAMP) or pathogen-associated molecular patterns (PAMP). It involves the formation of a protein complex with the pyrin domain of apoptosis-associated speck-like protein (ASC), the DAMP/PAMP, and the NLR/ALR. The protein complex activates caspase-1 downstream, releasing inflammatory cytokines and pyroptotic cell death (17,18). The pathogen- and host-derived triggers stimulate canonical NLRP3 inflammasomes, unlike other NLR-based inflammasomes. The NLRP3 and AIM2 inflammasomes are also expressed by proliferating keratinocytes within psoriatic skin. However, the role of macrophages in the long-term persistence of psoriasis inflammation locally and systemically has been neglected. The persistence of psoriatic lesions is largely caused due to significantly higher expression of macrophage inducible C-type lectin (mincle) associated with pro-inflammatory macrophages (M1) (19). Prokineticin-2 (PK2) is a neuroendocrine peptide associated with psoriatic skin (5,20). The PK2 activates NLRP3 inflammasomes in macrophages as an innate immune response, releasing IL-1β and exacerbating keratinocyte proliferation angiogenesis and psoriatic lesions (20,21). Macrophages are players in sensing the IL-1βand maintaining the PK-2-IL-1βpositive feedback loop (20). The tissue-resident macrophages (Langerhans cells) exist as a lineage from bone marrow from which the majority are lost due to disappearance reactions during the initial stages of the disease. Later, constant proliferation of remaining Langerhans cells occurs in response to persistent psoriatic inflammation (22). The tissue-resident macrophages are present since birth and multiply due to local triggers (DAMP/PAMP) instead of recruitment from the blood circulation, unlike the dendritic cells (22). Blood monocytes sense the inflammatory cytokine and circulate systemically causing vasculitis (22,23). Therefore, it is needed to stop the proliferation of the long-ignored tissue-resident macrophages to attenuate local inflammation. Given their enhanced activity and role in the pathogenesis of psoriasis, attenuating inflammasome activation within macrophages will serve as a novel therapy for psoriasis treatment. However, existing inflammasome inhibitors lack specificity, potency, and ability to cross the hydrophobic stratum corneum, reducing efficacy (24). Additionally, simultaneously targeting and inhibiting multiple types of inflammasomes can lead to enhanced efficacy against psoriasis. Nanoparticles have been utilized to enhance the cellular delivery of drugs for inflammatory diseases (25,26). Nanoparticles loaded with inflammasome inhibitor can provide efficacious inhibition and alleviate psoriasis symptoms.

Lipid nanoparticles (LNPs) have been explored for topical delivery of drugs for psoriasis treatment (27,28). However, recent reports demonstrated that LNPs consisting of phosphatidylcholine and PEGylated lipids, with or without cationic and ionizable lipids, activate inflammasomes or the immune response (29,30). Studies have also explored the effect of physicochemical properties of nanoparticles, including particle size, zeta potential, and formulation composition, on NLRP3 inflammasome activation (31,32). There are reports elaborating on the effect of LNPs on NLRP3 inflammasomes and toll-like receptors-2/4 (TLR-2/TLR-4) when the LNPs were modified for their chemical structure and surface charge (33-36). These studies indicate the inflammatory nature of lipidic carriers and hence, the challenges associated with utilizing conventional LNPs that could be inflammatory in nature and counterproductive for effective inflammasome inhibition and the delivery of inflammasome inhibitors. Therefore, LNPs were designed with self-inflammasome-inhibiting lipids and loaded with inflammasome inhibitors for synergistic inflammasome attenuation in macrophages. Dendritic cells and macrophages (Langerhans cells) are distinguished largely based on their function. The dendritic cells are a key player in the activation of adaptive immunity by presenting PAMP/DAMP to the T cells. On the contrary, macrophages are phagocytic cells. Therefore, the LNPs would be first phagocytosed by the macrophages. Inhibiting the inflammasomes in macrophages residing within psoriatic skin using LNPs would convert the pro-inflammatory phenotype (MI) into anti-inflammatory (M2) macrophages thereby, inhibiting chemokine and cytokine release, and other immune cells, which would serve as a negative feedback signal (37).

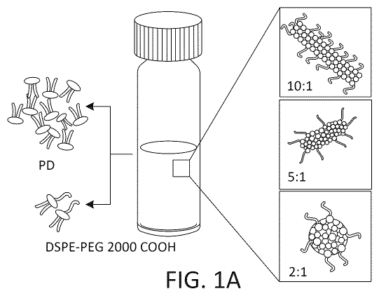

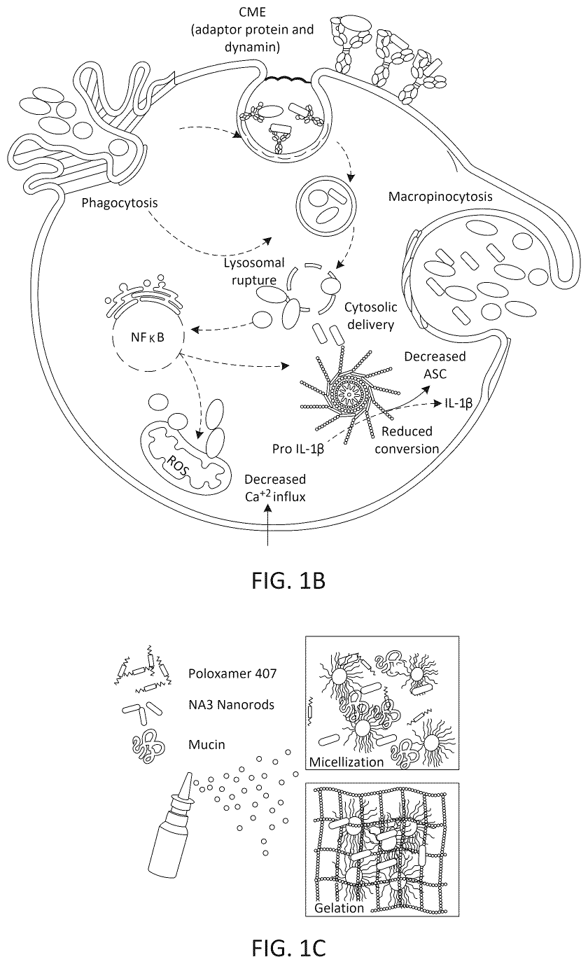

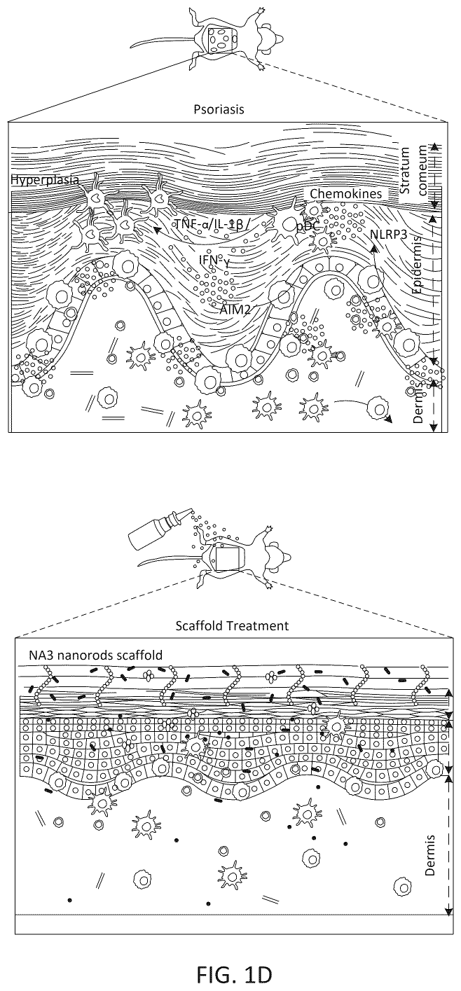

Inspired by the recent finding that non-spherical pathogens elicit enhanced adherence to macrophages (38,39), scientists engineered non-spherical (nanorods and nanoellipses) and spherical LNPs using inflammasome inhibitory lipids (trojan horse) (FIG. 1a) and studied their inflammasome inhibitory efficacy in macrophages. The study involved the development of non-spherical LNP with different shapes (nano-spheres/ellipses/rods) to evaluate the impact of shapes on enhanced skin penetration, macrophage adherence, recognition, specificity, and their efficacy in inhibiting inflammasome. To understand their mechanism of action, various internalization pathways of the non-spherical LNPs were investigated in macrophages (FIG. 1b) and their effect on cellular targets, thereby inhibiting inflammasomes (FIG. 1b). It was observed that the nanoellipses/rods were internalized by macropinocytosis and clathrin-mediated endocytosis, while the nanospheres were majorly phagocytosed. The receptors involved in cellular internalization varied based on the shape of the non-spherical LNP. It was also observed that the inhibition of the NLRP3 inflammasomes was significantly higher in macrophages treated with nanorods compared with nanoellipses and nanospheres. Therefore, nanorods were further utilized to develop an efficacious NLRP3-inhibiting anti-psoriatic drug delivery platform.

Recently, hyaluronic acid nanoparticles were demonstrated to possess an anti-psoriatic effect (40). Various synthetic or plant extracts inhibiting NLRP3/AIM2 inflammasome were also explored against psoriasis (41-45). However, the inhibitors and the hyaluronic acid nanoparticles would elicit systemic immunosuppressive effects due to intravenous or intragastric administration or be water soluble and may not penetrate the stratum corneum. To overcome these challenges, the present work involved the development of a sprayable polymeric scaffold loaded with lipid nanorods for localized delivery with enhanced skin penetration. Moreover, a synergistic effect was targeted by incorporating an NLRP3 and AIM2 dual inhibitor (NLRP3/AIM2-IN-3; NA3) in NLRP3 inflammasome inhibiting lipid nanorods, delivered topically. Poloxamer-407 (thermogelling agent) and mucin (thickening agent) were utilized to develop a topical spray (FIG. 1c). Mucin was shown to downregulate TLR-4 receptors and inhibit NLRP3 inflammasomes as per previous reports (46). Here, the synergy of NA3, trojan-horse nanorods, and mucin is showcased, which caused dual NLRP3- and AIM2-inflammasome inhibition in the Imiquimod (IMQ)-induced psoriasis-like mice model (FIG. 1d). The NA3-loaded nanorods delayed lysosomal rupture, and elicited NLRP3 and AIM2 inflammasome inhibition with reduced ASC speck formation. Interestingly, the sprayable NA3 nanorod-loaded polymeric scaffolds elicited an anti-psoriatic effect by localized reduction of CXCL2, NLRP3-, AIM2 inflammasome, IL-1β, Caspase-1, Gasdermin-D, TLR-7, and TLR-8 RNA levels along with IL-17A protein expression level in vivo in IMQ-induced psoriasis-like mice model. In summary, the sprayable NA3 nanorod-loaded polymeric scaffold served as a novel yet simple platform for localized anti-psoriatic delivery, elicited synergy by targeting NLRP3 and AIM2 dual inhibition, and avoided harmful systemic effects.

### Materials & Methods

### Materials/Reagents

Pyridoxine-3,4-Dipalmitate (PD) and 1,2-distearoyl-sn-glycero-3-phosphocthanolamine-N-(carboxyt (polyethylene glycol)-2000| (sodium salt) (DSPE-PEG (2000) carboxylic acid) were procured from Tokyo Chemical Industry America™ (Portland, USA), and Avanti Polar Lipids Inc. (Alabama, USA), iBMDM was a kind gift sample from Fitzgerald Lab, UMass Medical School, Worcester. Dulbecco's Modified Eagles Medium, Penicillin streptomycin, Fetal bovine serum (FBS), Trypan Blue, and Hank's balanced salt solution 1× from Gibco, Life Technologies (Billings, USA), Lipopolysaccharide (LPS) and Nigericin were procured from Invivogen and Sigma Aldrich, respectively. Poly (dA: dT) was purchased from Invivogen. Sulfuric acid was procured from Macron Chemicals (Radnor, USA), and a 96-well Corning ELISA plate was procured from Corning Incorporation (Durham, USA). IL-1βmouse uncoated ELISA kit, (5-(and-6)-Carboxyfluorescein Diacetate, Succinimidyl Ester) (CSFE), 1,1′-Diocatdecyl-3,3,3′,3′-Tetramethylindodicarbocyanine, 4-chlorobenzene sulfonate salt (DiD), MitoSox Red, Fluo-4 AM, Lyso Tracker Red DND 99, NucBlue™ Live ready probes™ Reagent (Hoechst 3342), and Trizol reagent were procured from Invitrogen Corporation (Waltham, USA). 96- and 12-well sterile treated plates were procured from Falcon (Glendale, USA). Phosphate Buffer Saline (PBS; 10×), Methylene dichloride, and Chlorpromazine hydrochloride (CPM) were purchased from ThermoScientific (Waltham, MA). Dynasore and Nocodazole from Selleckchem (Houston, USA), 5-(N-ethyl-N-isopropyl)-Amiloride (EIPA), and Cytochalasin D were obtained from Cayman Chemicals (Michigan, USA), and Sigma Aldrich Co. (St. Louis, USA), Cell-culture DMSO and 1-(4,5-Dimethyl-2-thiazolyl)-3,5-diphenyl formazan (MTT) was obtained from Fisher Biorcagents (Wisconsin, USA) and Millipore Corp. (Burlington, USA), respectively. FITC anti-mouse CD14 antibody was procured from Biolegend (San Diego, USA). High-Capacity cDNA reverse transcriptase kit and TaqMan Fast Advanced Master Mix were procured from Applied Biosystems. The primers of B-actin, Caspase-1, NLRP3, and AIM2 inflammasome, TLR4/7/8, Gasdermin-D, CXCL2, and IL-1βwere procured from Applied Biosystems. The Western blot antibodies for IL-17A and anti-mouse B-actin were procured from Cell Signaling Technology (Denver, USA) and caspase-1 was obtained from Adipogen Life Sciences (San Diego, USA). Anti-mouse and antirabbit IgG conjugated to HRP was procured from Cell Signaling Technology (Denver, USA). The RIPA lysis buffer, and halt protease & phosphatase inhibitor cocktail (100×) were purchased from ThermoScientific. The TGX strain-free FastCast Acrylamide Kit, 10%, and Clarity One ECL substrate were purchased from Biorad (California, USA). The TRIZMA base, sodium dodecyl sulfate, glycine, sodium chloride, tween 20, bovine serum albumin (BSA), methanol, and 2-mercaptoethanol were procured from Sigma Aldrich (St.Louis, USA). The DAB Ki67 and CD31 antibody was procured from Abcam (Waltham, MA). The Balb/c mice for in-vivo studies were obtained from Charles River Laboratories (Protocol no. 4895).

Formula for NLRP3/AIM2-IN-3 (NA3):

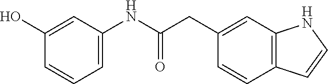 [SMILES: O=C(Cc1ccc2cc[nH]c2c1)Nc1cccc(O)c1]

(CAS No.: 1787787-60-3). Other inhibitors than can be used in the practice of the invention are NLRP3 Inflammasome Inhibitor I. MCC950. BMS986299. BAL-0028. Disulfuram or a combination thereof.

TABLE 1
qPCR primers

| Species | Gene | Assay ID |
|---|---|---|
| Mouse | B-actin | Mm02619580_g1 |
| | NLRP3 | Mm00840904_m1 |
| | IL-1β | Mm01336189_m1 |
| | AIM2 | Mm01295719_m1 |
| | TLR4 | Mm00445273_m1 |
| | CXCL2 | Mm00436450_m1 |
| | Caspase-1 | Mm00438023_m1 |
| | Gasdermin-D | Mm00509958_m1 |
| | TLR7 | Mm00446590_m1 |
| | TLR8 | Mm04209873_m1 |

TABLE 2
Mice study groups for in vivo efficacy
studies in IMQ-induced psoriasis

| Sr. | | |
|---|---|---|
| no | Animal group | Treatment |
| 1 | Negative control | — |
| 2 | Positive control | Imiquimod (5 days) |
| 3 | Only Nanorod | Imiquimod (5 days) + Blank nanorods (equivalent |
| | treatment | to 800 μM NA3) + gelling agent (10% w/v) |
| | | (5 days) |
| 4 | NA3 Nanorods | Imiquimod (5 days) + NA3 loaded nanorods |
| | | (equivalent to 800 μM NA3) + Poloxamer 407 |
| | | (10% w/v) + mucin (5 days) |

TABLE 3
PASI scoring

| Sr no | Severity | Score |
|---|---|---|
| 1 | None | 0 |
| 2 | Mild | 1 |
| 3 | Moderate | 2 |
| 4 | Severe | 3 |
| 5 | Very severe | 4 |

### Results

### Synthesis and Characterization of Nano-Spheres/Ellipses/Rods

It is established that PRR, including but not limited to TLR-4 and cluster of differentiation 14 (CD14), recognize PAMP and stimulate NLRP3 inflammasome activation (47). The pathogens are differently shaped, including spherical and ellipsoidal cocci or rod- and spiral or flagellated bacilli (48). The rod-shaped pathogens cling to the macrophage surface and enhance internalization compared to non-spherical pathogens (38). It was hypothesized that the shape of the pathogens could be recognized and reminisced by the macrophage receptors and the internalization pathway would differ for non-spherical pathogens compared to spherical ones. Therefore, the objective involved developing pathogen-mimicking lipid-based nano-spheres, ellipses, and rods to study their effect on macrophage targeting, internalization pathway, and inflammasomes inhibition (FIG. 1a,b). Activated inflammasomes are implicated in psoriasis pathogenesis and progression (49). Pyridoxine (Vitamin B6) is a well-known inflammasome inhibitor and can be utilized as a part of a lipid such as pyridoxine dipalmitate (PD) to facilitate the self-assembly to lipid nanoparticles (50). Nanorods encompassing PD were developed and transformed into a topical anti-inflammatory formulation eliciting enhanced internalization and targeted delivery to macrophages. It was rationalized that the nanorods containing PD would result in trojan-horse pathogen-like LNPs and elicit an anti-inflammatory response.

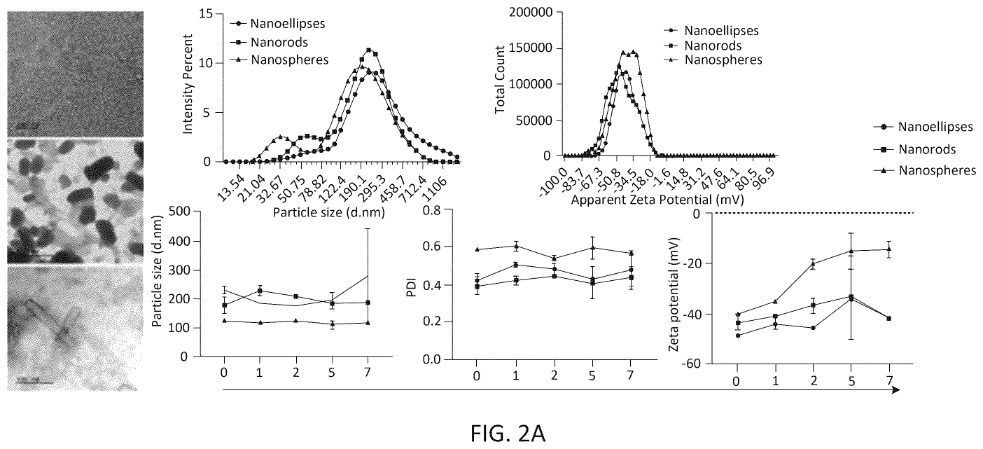

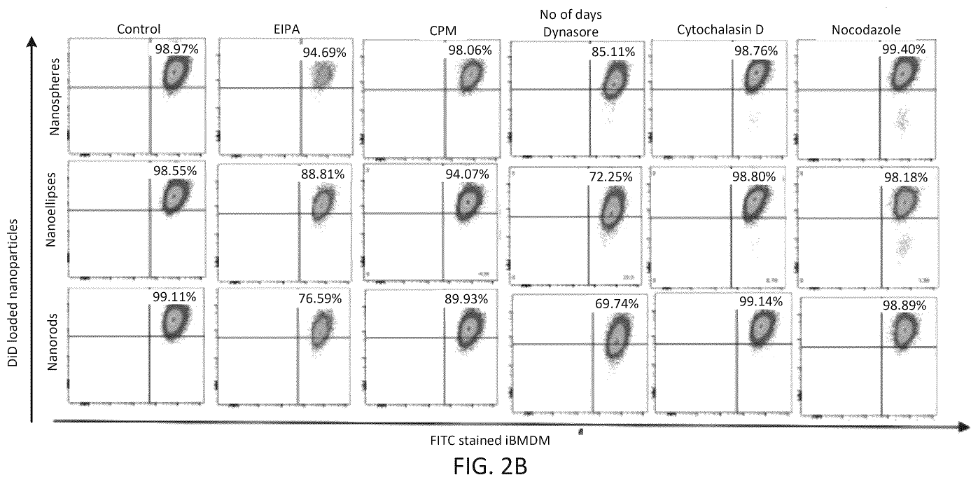

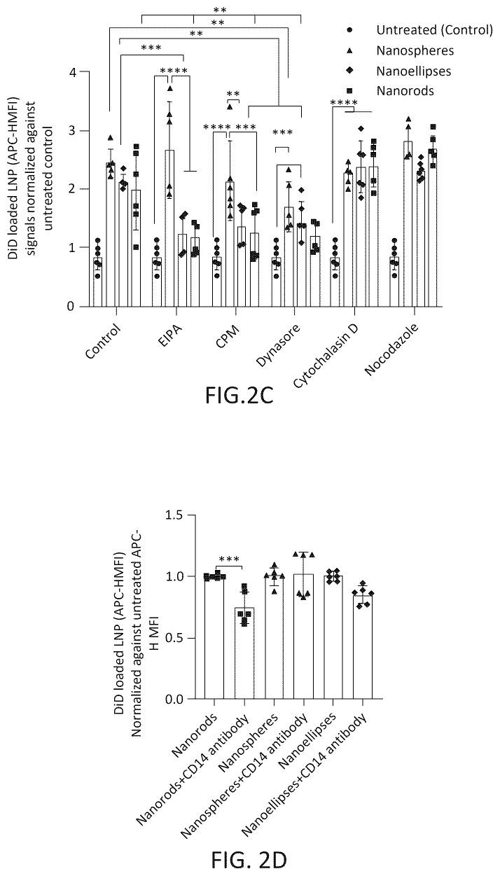

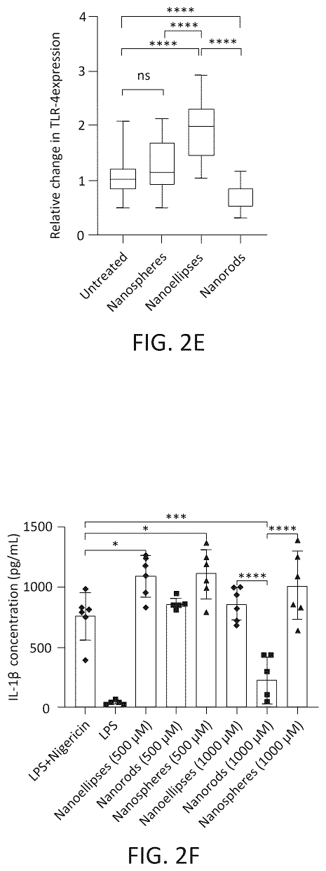

First, self-assembled nanoparticles with increasing aspect ratio from spheres to ellipses and rods were developed by using anti-inflammatory PD and DSPE-PEG (2000) carboxylic acid as a stabilizing phospholipid to study the effect of nanoparticle shape on inflammasomes (50,51). Upon increasing the molar ratio of PD: DSPE-PEG (2000) carboxylic acid from 2:1 to 5:1 and 10:1 increased the aspect ratio of the nanoparticle leading to the formation of nanospheres (124.70+0.43 nm), nanoellipses (231.85±12.37 nm), and nanorods (176.6±28.443 nm), respectively (FIG. 2a). It is hypothesized that the increased hydrophobicity of the core and lower curvature of PD in the case of nanoellipses and nanorods, along with the repulsive forces of the PEG chain, led to an increase in the aspect ratio of the nanoparticle to form nanoellipses and nanorods (52). The non-spherical morphology of the LNPs would have reduced Gibb's free energy and enhanced the thermodynamic stability of the system (53,54). A similar effect was observed previously when the molar ratio of ascorbyl dipalmitate increased the aspect ratio of the nanoparticles to reduce the Gibbs free energy of mixing (52,55,56). Interestingly, bimodal particle size distribution was observed for nanospheres compared with unimodal particle size distribution for nanoellipses and nanorods (FIG. 2a). The bimodal distribution of nanospheres correlated with previous reports when ascorbyl dipalmitate and DSPE-PEG 2000 were used in 0.125:1 molar ratio (52). The particle size distribution graph corroborated with the polydispersity index (PDI). The PDI decreased with an increase in the nanoparticle aspect ratio from nanospheres (0.5805±0.006) compared with nanoellipses (0.422±0.039) and nanorods (0.392±0.048), respectively. Furthermore, the particle size and PDI of the nanospheres, nanoellipses, and nanorods remained stable for up to 7 days at 4° C. (FIG. 2a). On the contrary, the zeta potential of the nanospheres increased 2-fold on day 1 and remained stable until day 7. The zeta potential for nanoellipses and nanorods remained consistent until day 7.

### Evaluation of Cellular Internalization Pathway of Nano-Spheres/Ellipses/Rods.

DiD-loaded nano-spheres/ellipses/rods were developed to unveil the nanoparticle shape-memory effect of PRR and the mechanism of cellular internalization in immortal bone marrow-derived macrophages (iBMDMs). PD, DSPE-PEG 2000 carboxylic acid, and DID (0.067% w/w) were dissolved in dichloromethane (DCM) and evaporated to form a thin film. The thin film was rehydrated using Milli-Q to form the DiD-loaded nano-spheres/ellipses/rods. The DiD-loaded nano-spheres/ellipses/rods were internalized efficiently by the iBMDMs (FIGS. 2b and c). Apart from phagocytosis, various internalization pathways exist in iBMDM cells, including macropinocytosis, caveolae-, and clathrin-mediated endocytosis (57). The mean fluorescence intensity of DiD-loaded nano-spheres, ellipses, and rods was measured in iBMDM pretreated with macropinocytosis, phagocytosis, clathrin-(CME), dynamin-dependent endocytosis inhibitors to elucidate the cellular internalization pathway. It was observed that the internalization decreased with an increase in an aspect ratio of nanoparticles from spheres to ellipses and rods in iBMDMs pretreated with EIPA, CPM, and Dynasore inhibiting macropinocytosis, CME, and dynamin-dependent endocytosis, respectively. The cellular uptake of DiD nanoellipses was 3.2-, 1.9-, and 1.6-fold lower compared with nanospheres in iBMDMs pretreated with EIPA, CPM, and Dynasore (FIGS. 2b and c). Whereas the DiD-loaded nanorods depicted reduced internalization by 3.2-, 2.3-, and 2.03-folds compared with nanospheres in iBMDMs pretreated with EIPA, CPM, and Dynasore, respectively (FIG. 2b-c). On the contrary, the cellular uptake was unaffected by the pre-treatment of cytochalasin D 10 (actin polymerization inhibitor affecting phagocytosis) and Nocodazole (microtubule and spindle formation inhibitor) (FIG. 2c) These results demonstrated that the nanoellipses and nanorods were internalized primarily by macropinocytosis and clathrin-mediated endocytosis in iBMDMs due to their significant reduction in internalization in the presence of EIPA (p<0.0001) and CPM (p<0.001 and 0.01), respectively. In contrast, the nanospheres were internalized majorly by phagocytosis due to their reduced internalization in iBMDMs pretreated with Dynasore. The clathrin pits endocytose the receptors; therefore, the non-spherical LNPs would be recognized through specific receptors before endocytosis through clathrin pits.

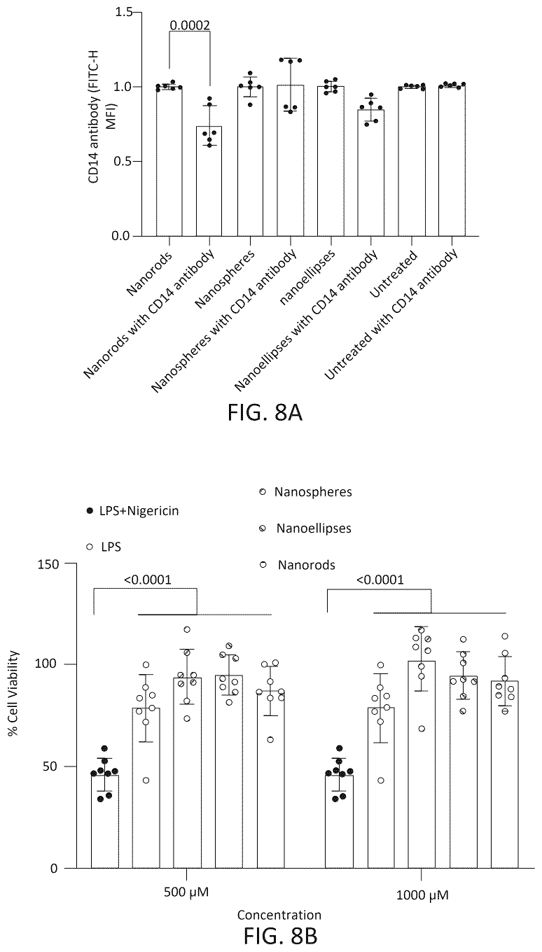

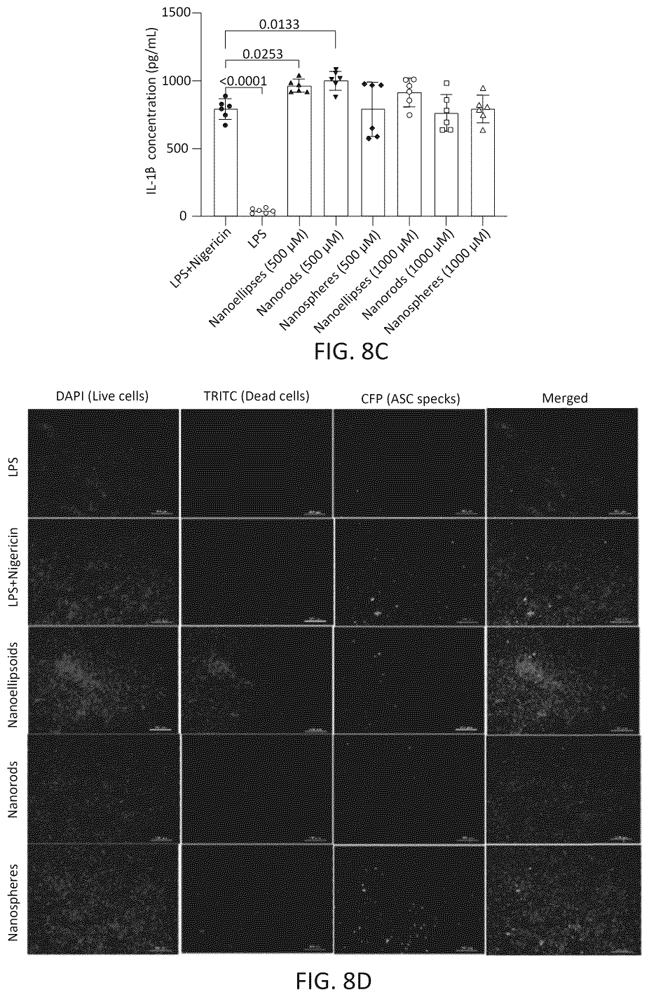

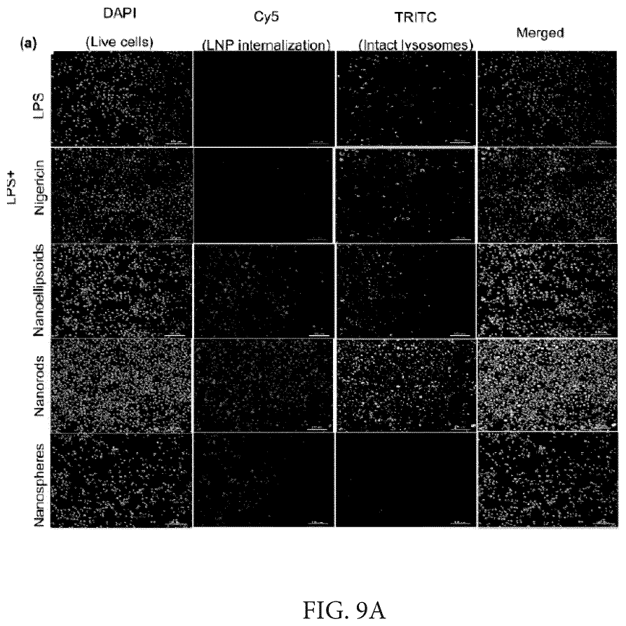

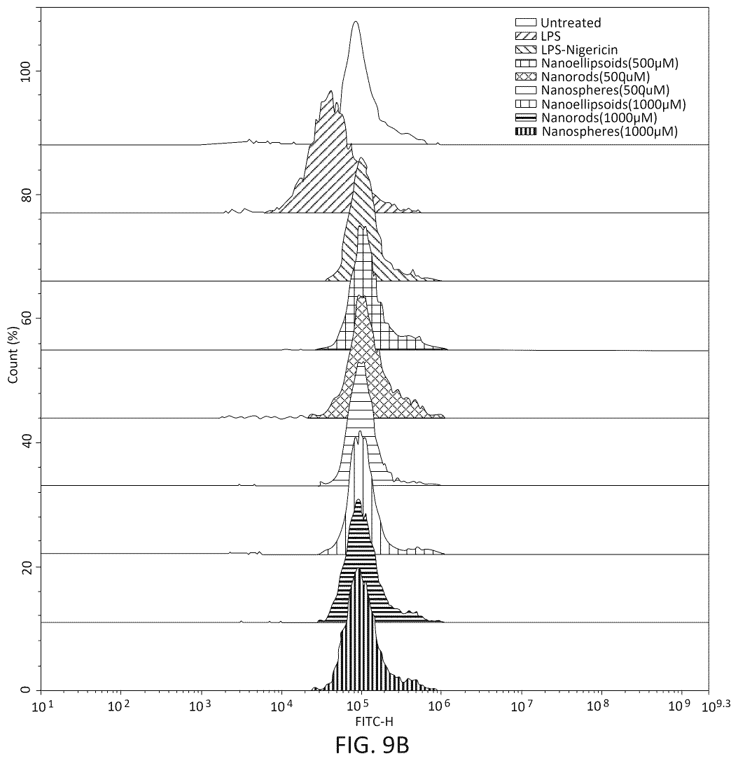

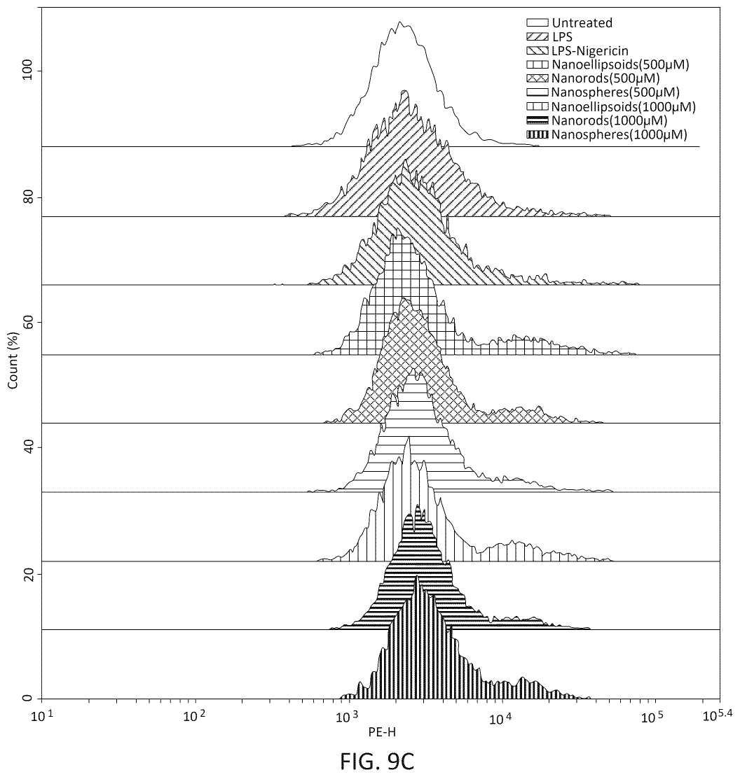

CD14 protein recognizes damage-associated molecular protein (DAMP) and pathogen-associated molecular protein (PAMP), activating canonical and non-canonical inflammasome pathways (58). The literature revealed that lipopolysaccharide (LPS; PAMP) is recognized by both CD14 and TLR-4, while oxidized phosphorylcholine (ox-PAPC) derivatives (DAMP) are recognized only by CD14 without the involvement of TLR-4 activating the NLRP3 inflammasome (59). Therefore, the involvement of the CD14 and TLR-4 in the internalization of nano-spheres/ellipses/rods was further explored. The CD14 receptor was blocked using anti-CD14 fluorescein isothiocyanate (FITC) antibody. The FITC signals increased significantly in anti-CD14 antibody treated iBMDMs (FIG. 8a). Interestingly, the internalization of nanorods was significantly reduced by 1.36- and 1.2-fold compared with nano-spheres (p<0.0001) and nanoellipses, respectively when the CD14 receptors were blocked using anti-CD14 FITC antibody. Moreover, the internalization of nanorods was decreased significantly (p<0.001) compared with iBMDMs without CD14 FITC antibody treated group (FIG. 2d). Therefore, the CD14 receptors could exclusively recognize the non-spherical nanorods compared with nano-spheres and nano-ellipses. Furthermore, it was observed that the relative expression of TLR-4 increased in iBMDMs treated with nanoellipses compared with nanorods (p<0.0001) (FIG. 2e). These results demonstrated that the nanoellipses were internalized through CD14 and TLR-4, whereas the internalization of nanorods occurred only through the CD14 receptor. In comparison, the expression of CD14 and TLR-4 remained unaffected in iBMDMs treated with nanospheres, which were primarily phagocytosed.

Cytotoxicity studies of nano-spheres/ellipses/rods by MTT assay.

The cellular IL-1βinflammatory cytokine levels largely depend upon cellular viability. IL-1βbinds to IL-1 receptors on the surface of immune cells and triggers further inflammatory processes (60). However, prolonged NLRP3 inflammasome activation expedites pyroptosis, which might diminish IL-1βlevels (61). The Trojan-horse nanoparticles were anticipated to elicit an inhibitory effect on NLRP3 inflammasomes without causing cytotoxicity. The cytotoxic effect of the nanospheres, ellipses, and rods in iBMDMs was determined by MTT assay. Combined activation by LPS (signal 1) and Nigericin (Signal 2) caused pyroptosis with 45.86±7.2% iBMDM cell viability. On the contrary, the cell viability was significantly higher (p<0.0001) in iBMDMs treated with LPS (90.07±19.12%), and 500 μM or 1000 μM of nano-spheres (94.0±13% and 100±20%), ellipses (95.0±10% and 94.64±11.6%), and rods (87.15±11.8% and 92.03±11.9%) compared with LPS and Nigericin treated iBMDMs (FIG. 8b). Therefore, the nano-spheres, ellipses, and rods at 500 μM and 1000 μM concentration levels were considered safe for iBMDMs.

Effect of nano-spheres/ellipses/rods on in vitro NLRP3 inflammasome inhibition.

The inhibitory effect of the nanoparticles on NLRP3 inflammasomes in the LPS and Nigericin (positive control) (62) activated iBMDMs would be evident with reduced IL-1βlevels. It was observed that nanospheres and nanoellipses (500 μM and 1000 μM) did not inhibit the inflammasome activation in Nigericin-treated iBMDMs (FIG. 2f). Moreover, the inhibitory effect of the nanorods diminished in iBMDMs activated with LPS (FIG. 8c). The LPS is known to bind the TLR4 along with co-receptor CD14 and furnishes signal 1 for NLRP3 inflammasome activation (47). It was reported that oxidized phospholipid 1-palmitoyl-2-13 arachidonoyl-sn-glycero-3-phosphorylcholine (ox-PAPC, cellular DAMP) utilizes CD14 to internalize within the cells and activate NLRP3 inflammasomes (59). Therefore, based on the nanorods' cellular internalization mechanism involving LPS recognizing CD14 receptor protein (FIG. 2d), it is hypothesized that nanorods might mimic the ox-PAPC as trojan horse and successfully inhibit NLRP3 inflammasomes.

Significant concentration dependent NLRP3 inflammasome inhibition was observed when iBMDM was treated with nanorods followed by LPS. Also, 1.3-fold (500 μM) and 3.8-fold (1000 μM) NLRP3 inflammasome attenuation was observed in nanorods pretreated iBMDM compared with nanoellipses (FIG. 2f). To conclude, the aspect ratio of the trojan-horse nanoparticles was directly proportional to the extent of NLRP3 inflammasome inhibition in iBMDMs. Moreover, upregulation of TLR-4 RNA expression in nanoellipse-treated iBMDMs, as observed previously (FIG. 2e), would have resulted in lower NLRP3 inflammasome inhibition than nanorod-treated iBMDMs. Additionally, Pyridoxine elicits an antioxidant and anti-inflammatory effect. Therefore, the reduced IL-1βlevel caused by the trojan-horse nanorods could be attributed to higher PD compared with nano-spheres and ellipses (50). These results elucidate further the various mechanisms resulting in the inhibitory effect on NLRP3 inflammasomes. Greater adherence of the nanorods onto the macrophage might diminish protracted LPS-induced signal 1 stimulation required for activation of NLRP3 inflammasome (63,64).

Effect of nano-spheres/ellipses/rods on ASC speck and lysosome.

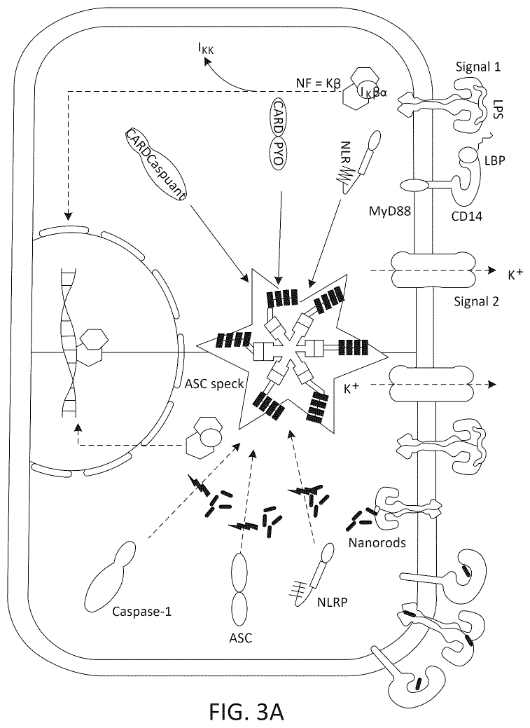

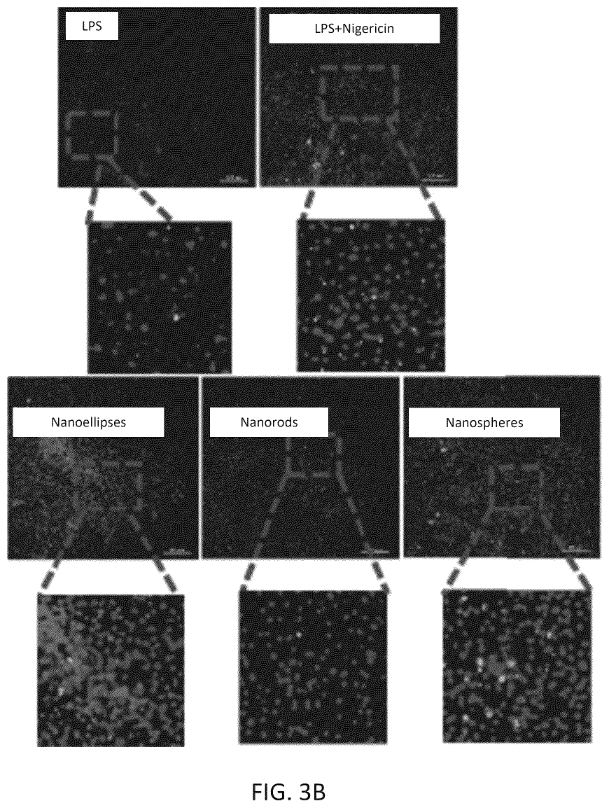

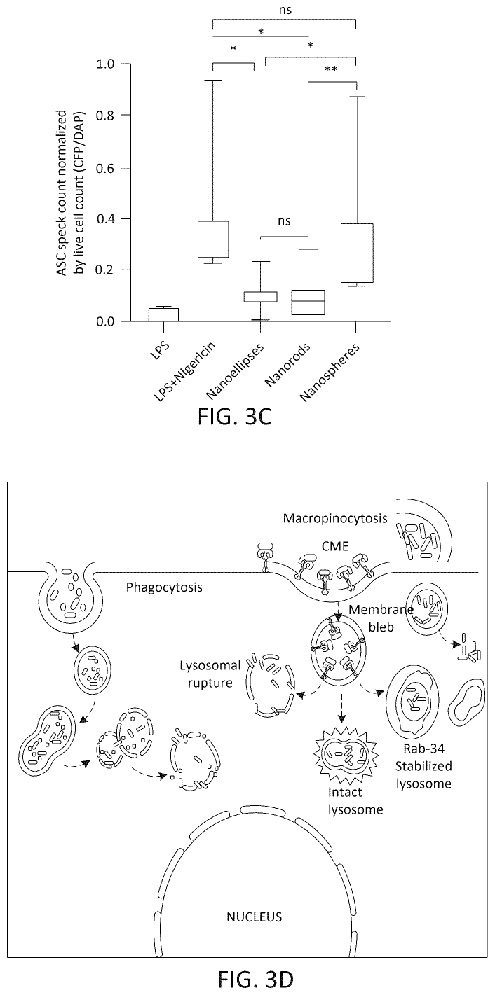

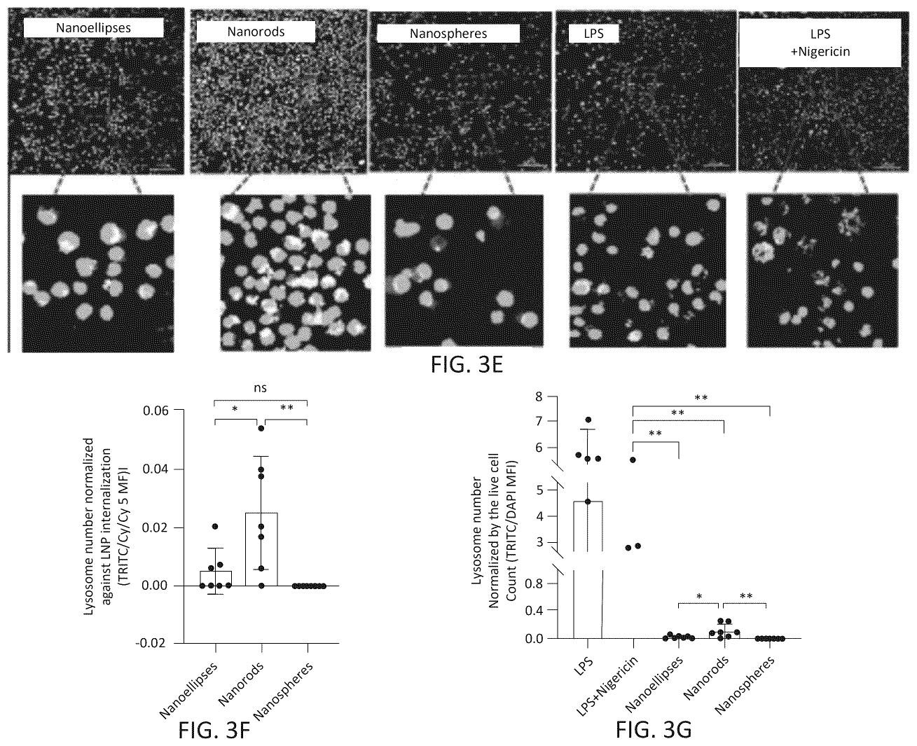

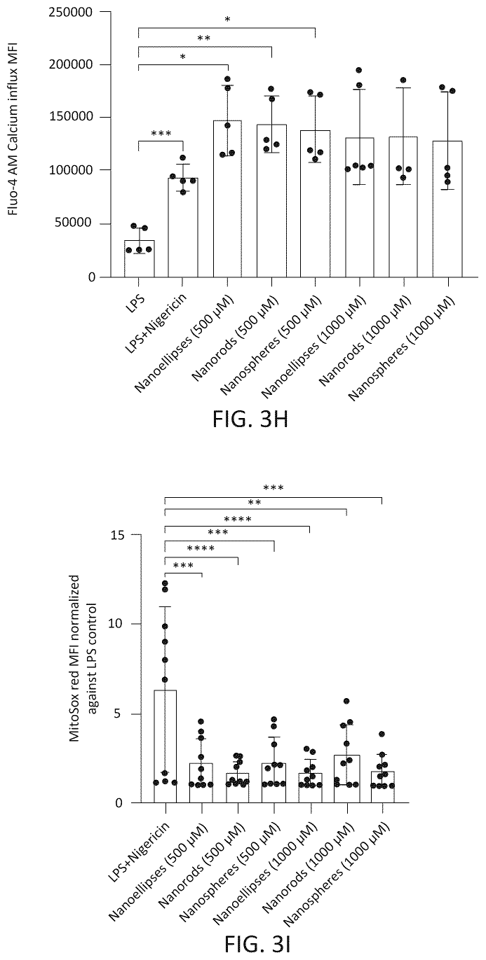

The NLRP3 inflammasome is a multi-protein complex that when activated causes oligomerization of adaptor-protein apoptosis-associated speck-like protein containing pyrin domain (ASC) and causes caspase activation and recruiting domain (CARD) (65). The ASC further oligomerizes and activates pro-caspase-1, which upon auto-proteolysis cleaves to caspase-1. Caspase-1 stimulates the release of cytokines, gasdermin D pore formation, and pyroptosis (61). After that, the released ASC and caspase-1 further activate the NLRP3 inflammasomes in other cells (FIG. 3a) (66). Given the significance of ASC in NLRP3 inflammasome activation, an orthogonal technique of determining the ASC speck formation could further confirm the ability of the nano-spheres/ellipses/rods to inhibit the NLRP3 inflammasomes. The number of ASC specks per live cell was significantly reduced in iBMDMs pretreated with nanoellipses (p<0.01) and nanorods (p<0.001) compared with iBMDMs treated with positive control (FIGS. 3b and c). Moreover, no significant difference was observed in the 14 iBMDMs treated with nanospheres compared with positive control. The ASC speck per live cell was 3.2- and 11.5-fold higher in iBMDMs treated with nanospheres compared with nanoellipses (p<0.001) and nanorods (p<0.0001), respectively. Therefore, increasing the aspect ratio of the nanoparticles effectively inhibits ASC speck formation required for downstream inflammatory signaling cascade.

The DAMP and nanoparticle associated molecular pattern (NAMP) are internalized and released into the cytosol by lysosomal rupture, triggering the oligomerization of ASC and NLRP3 activation (FIG. 3d) (32,61). The majority of bacteria (67,68) and viruses (69) endocytose, resulting in lysosomal rupture and cathepsin B release activating NLRP3 inflammasomes (70). Interestingly, the pathogen-like nanorods depicted reduced lysosomal rupture compared with nanoellipses (p<0.05) and nanospheres (p<0.01) (FIG. 3c-g). Although nano-ellipses and nanorods could reduce the ASC speck formation, only nanorods efficiently delayed lysosomal rupture compared with nano-ellipses which could cause significant impact on the inhibition of NLRP3 inflammasomes. Moreover, Nigericin is a well-known signal 2 that activates inflammasome without lysosomal rupture by increasing potassium efflux (71). Therefore, significantly higher intact lysosomes were observed in LPS and Nigericin-treated iBMDMs (FIG. 3g). Evasion of lysosomal rupture through membrane-bound blebs by macropinocytosis is one of the primary mechanisms for virus entry (72). The inhibition of lysosomal rupture observed in the case of nano-ellipses and rods compared with nanospheres could be attributed to their internalization through macropinocytosis (similar to the mechanism of virus entry) in addition to Clathrin-mediated endocytosis (FIG. 3d, FIG. 2c) as trojan horses.

Effect of nano-spheres/ellipses/rods on Calcium influx and mitochondrial reactive oxygen species.

Under ordinary conditions, the cytosolic calcium is significantly lower than the extracellular (<200,000-folds) and endoplasmic reticulum or lysosome (<5000-folds) (73). However, the membrane depolarization and endoplasmic reticulum/lysosomal membrane permeabilization cause a continuous inflow of calcium due to an electrochemical gradient (74). Since the developed trojan horse LNPs caused lysosomal membrane permeabilization, observing the effect on calcium influx would be noteworthy. It was observed that Nano-spheres, ellipses, and rods restrained the intracellular calcium in iBMDM cells similar to the LPS and Nigericin (positive control) treated iBMDMs. Potassium efflux is associated with calcium influx through the cell membrane channel (75). Nigericin is a known NLRP3 signal 2 molecule that increases potassium influx through the cellular membrane, resulting in calcium efflux upstream of NLRP3 inflammasomes. Therefore, the increase in calcium influx for Nano-spheres/ellipses/rods could be attributed to the combined effect of Nigericin-induced calcium influx through the cell membrane and the release of lysosomal calcium stores. (FIG. 3h).

Identification of DAMP, NAMP, or PAMP is associated with various cellular responses, including increased cell volume, membrane permeabilization, and mitochondrial ROS (mtROS) generation (76). Therefore, the effect of nano-spheres, ellipses, and rods was evaluated for their efficacy against mtROS generation. The nanospheres (p<0.001), nanoellipses (p<0.001 and 0.0001), and nanorods (p<0.0001 and 0.01) were effective in inhibiting the mtROS generation in iBMDMs by 2.3-3.9-folds when compared with positive control (FIG. 3i). Each group treated with the nano-spheres/ellipses/rods were activated by LPS (signal 1) and Nigericin (signal 2) and the efficacy of the nano-spheres/ellipses/rods to attenuate mtROS was determined. Nigericin elicits an effect on mtROS generation through NLRP3-dependent and stress-induced severe mitochondrial dysfunction (NLRP3 independent pathway) 77. However, the current study aimed to elucidate whether the effect of nano-spheres/ellipses/rods on mtROS generation was caused due to activated NLRP3 inflammasomes.

An increase in lysosomal membrane permeabilization leads to calcium efflux, signaling the endoplasmic reticulum and mitochondrion. This leads to the generation of mtROS production. However, PD is an antioxidant that could inhibit the ROS (50,78). Therefore, the increased mtROS inhibition observed in the case of nano-ellipses and spheres could be attributed to higher lysosomal rupture in iBMDMs treated with nano-ellipses and spheres, causing enhanced cytosolic PD concentration and mtROS inhibition. Therefore, the nanorods were efficacious compared with nano-spheres and ellipses in NLRP3 inflammasome attenuation due to decrease in TLR-4 upregulation, ASC speck formation, NLRP3 oligomerization, and lysosomal rupture.

NA3 nanorod-loaded scaffold design and characterization.

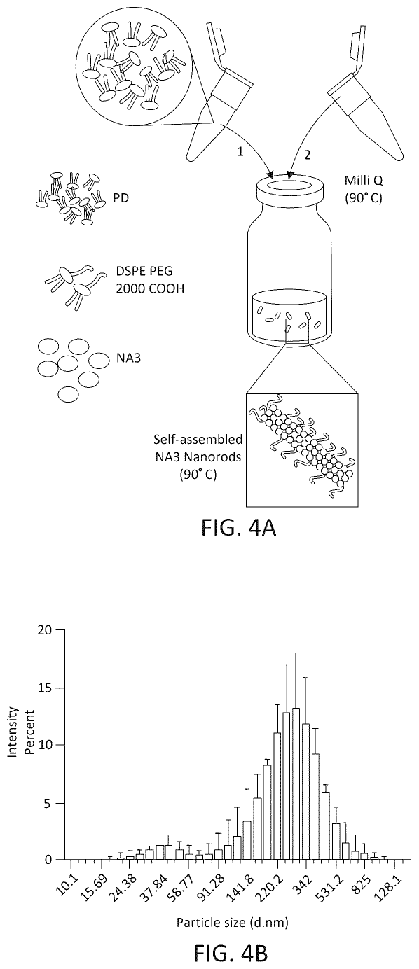

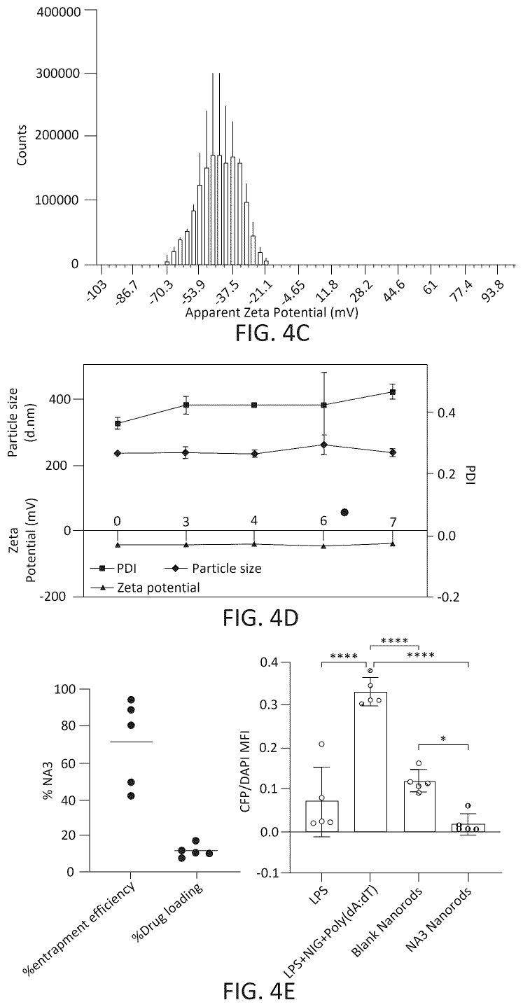

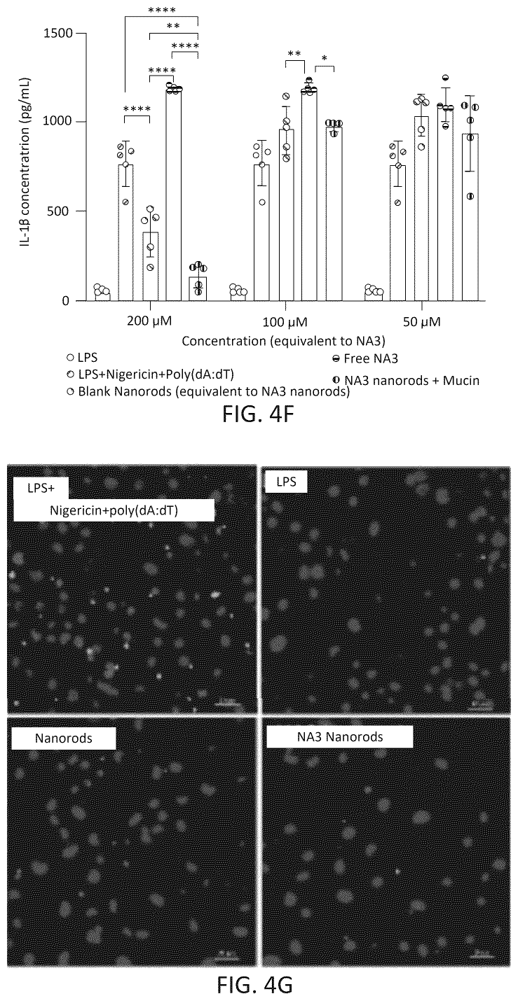

Exacerbated keratinocyte proliferation and psoriatic lesions are caused by the combination of NLRP3 and AIM2 inflammasomes (5,79). Administration of immunosuppressants would enhance the risk of comorbidities in the case of psoriasis (80). Therefore, localized simultaneous modulation of NLRP3 and AIM2 inflammasomes in macrophages of psoriatic skin coax safe recovery from psoriasis. The NA3 was loaded into the nanorods to enhance the efficacy toward both NLRP3 and AIM2 (FIG. 4a). The process parameters, including sonication time from 10 min to 30 mins and lipid concentrations such as 2:1 to 5:1 and 10:1, were optimized to yield stable NA3 nanorods (234.6±4.7 nm, 0.42±0.02,-43.3±1.3 mV) (FIGS. 4b and c). The NA3 nanorods were stable for 7 days at 4° C. (FIG. 4d) with the NA3 loading and % entrapment efficiency of 11.70%±3.5% and 71.11%+23.6%, respectively (FIG. 4c).

One study compared the efficacy of NA3 on NLRP3 and AIM2 inflammasomes in both J774A.1 cells and bone marrow-derived macrophages (BMDMs) (81). NA3 depicted lower efficacy on AIM2 inflammasomes in J774A.1 cells compared with BMDMs while the drug elicited significantly higher efficacy on NLRP3 inflammasomes in J774A. 1 cells (IC50-19.62 μM) compared with BMDMs (IC50-48.98 μM) (81). However, in presence of the Poly dA: dT (AIM2 stimulating signal 2) the NA3 loaded nanorods elicited enhanced efficacy (p<0.0001) against NLRP3 and AIM2 inflammasomes in iBMDMs compared with free NA3 and blank nanorods. It was observed that the in-vitro efficacy of the NA3 loaded nanorods (equivalent to 200 μM NA3) depicted 21.5- and 59-fold reduction in IL-1βlevels compared with blank nanorods and free NA3, respectively (FIG. 4f). Therefore, the efficacy of NA3 was improved compared with free NA3 when incorporate within nanorods. Since the activation of both NLRP3 and AIM2 results in ASC speck formation, the effect of NA3-loaded nanorods on ASC speck was studied further. The results corroborated with the IL-1βELISA with an 11.9-fold reduction in ASC speck formation compared with blank nanorods (FIG. 4g).

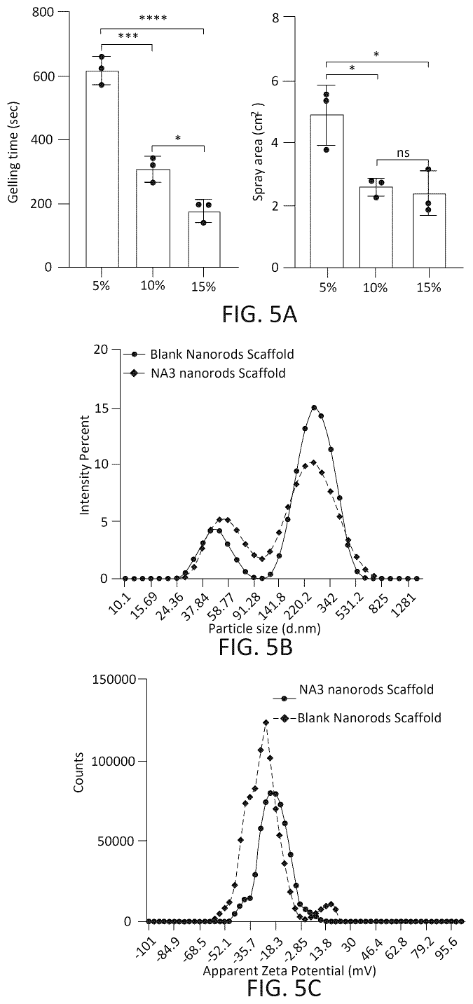

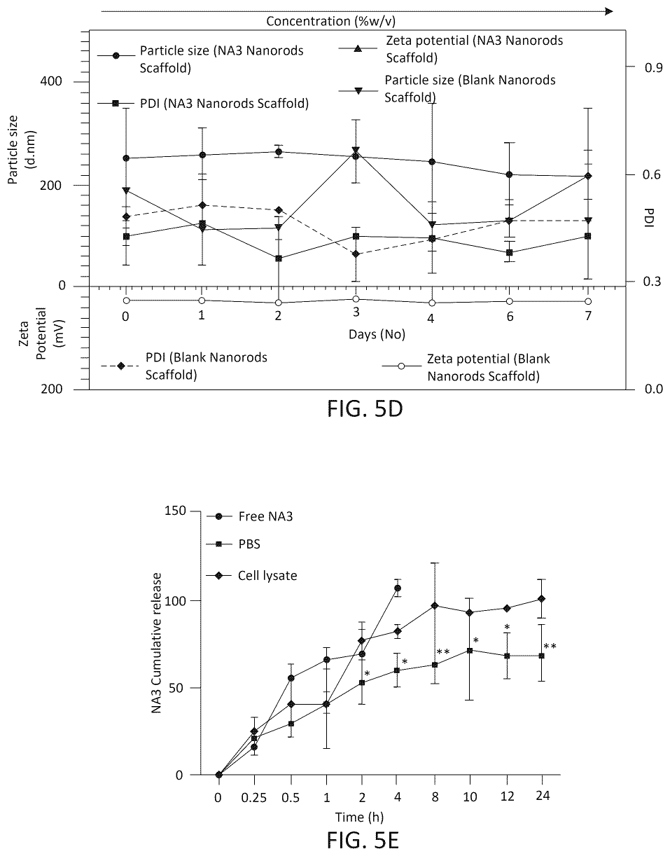

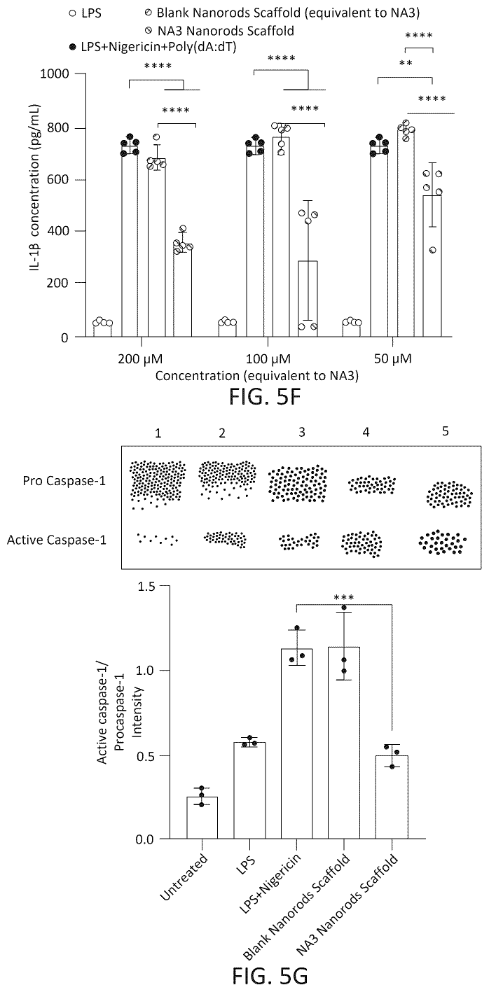

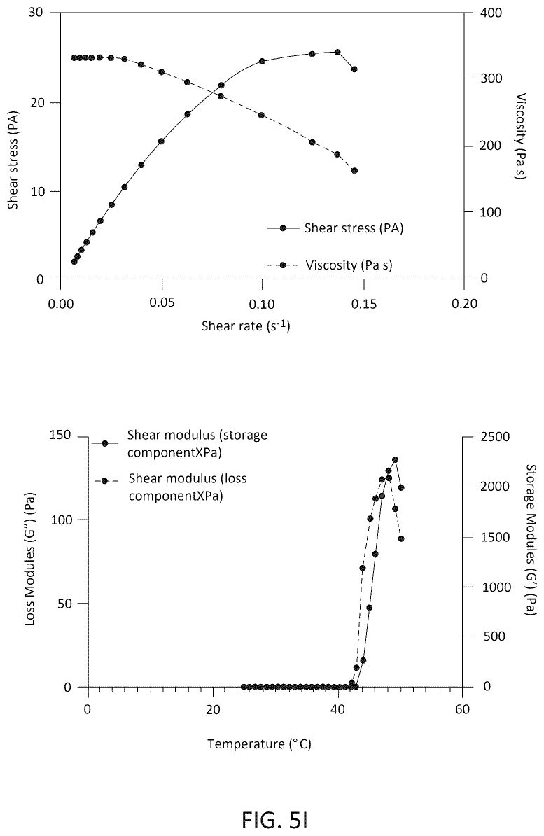

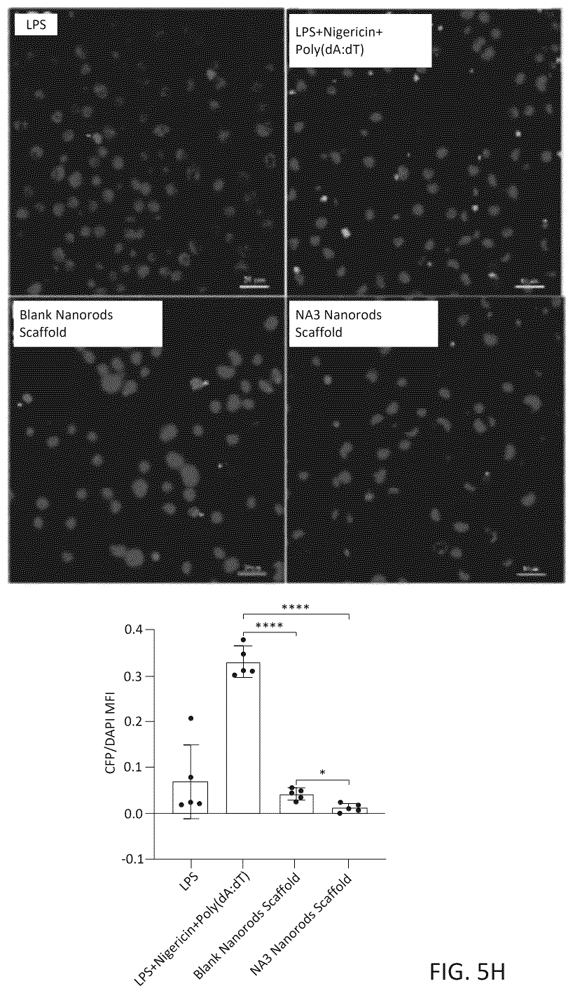

To facilitate the topical application of NA3-loaded nanorods, a thermogelling excipient was used. Poloxamer 407, due to the presence of poly(propylene oxide) (PPO) and poly(ethylene oxide) (PEO) groups, elicits micellization and gelation at 37° C. and ensures low toxicity due to its non-ionic nature (82,83). It is reported that Poloxamer 407 was used in topical formulations with up to 20% w/v concentration (84). It was observed that the gelling time decreased from 620 s±43.9 s to 177.3 s±35.8 s with increased Poloxamer 407 concentration (5%-15 w/v) (FIG. 5a). Similarly, the spray area of 5% w/v poloxamer solution was significantly higher (4.8±0.95 cm²) compared with 10% w/v (2.6±0.27 cm²) and 15% w/v (2.4±0.71 cm²) (p<0.05) (FIG. 5a). Considering the required consistency, a sprayable NA3-loaded scaffold containing 10% w/v Poloxamer 407 was utilized for topical administration. Mucin (2.5 mg/mL) was used as a thickening agent to enhance the viscosity further and improve the anti-inflammatory effect (FIG. 1c). The non-ionic surfactant property of Poloxamer 407 was predicated to enhance the penetration of the NA3 nanorods and hydrophilic mucin (FIGS. 1c and d) within the thickened psoriatic skin. The NA3 nanorod polymeric scaffold (252.75±95.8 nm, 0.426±0.02,−27.5±0 mV) and the blank nanorods polymeric scaffold (187.7±57.13 nm, 0.48±0.14,−26±3.7 mV) were stable for 7 days at 4° C. (FIG. 5b-d). The sprayable NA3 nanorod polymeric scaffold led to sustained NA3 release (69.09±16.27%) up to 24h in PBS (FIG. 5c). Furthermore, the drug release kinetics of NA3 nanorods polymeric scaffold was done in the presence of cell lysate.

The cumulative drug release was found to be 96.67±23.9% within 8 hours in cell lysate. Therefore, the NA3 nanorods would lead to quick release once within the macrophages. On the contrary, 107.1±4.5% NA3 was released within 4 hours in the case of free drug. The in vitro efficacy of NLRP-3 and AIM2 inhibition by sprayable NA3 nanorods polymeric scaffold was evaluated by IL-1βELISA in iBMDMs. Dual inhibition of NLRP3 and AIM2 inflammasomes in iBMDMs led to significantly lower IL-1β concentration (p<0.0001) compared with blank nanorods scaffold (FIG. 5f). Moreover, the NA3 nanorods scaffold (50-200 μM NA3 equivalent) caused 1.8-2.2-fold reduction IL-1βlevels directly correlating with reduced NLRP3 and AIM2 inflammasome activation compared with blank nanorods. In contrast, only a 1.5-fold reduction in IL-1βlevel indicating the NLRP3/AIM2 inflammasome reduction was observed in iBMDMs treated with blank nanorods scaffold (equivalent to 200 μM NA3) compared with the positive control (p<0.0001) (FIG. 5f). It was observed that nanorods caused downregulation of TLR-4 RNA expression, delayed lysosomal rupture, and ASC speck formation. However, their effect on active caspase-1 was not elucidated. The NA3 nanorod polymeric scaffold significantly reduced active caspase-1 formation compared with positive control (p<0.001). On the contrary, the blank nanorods did not have any effect on active caspase-1 (FIG. 5g). Therefore, the NA3 nanorod-loaded polymeric scaffold caused significantly lower IL-1βlevels resulting from decreased NLRP3 and AIM2 inflammasome formation compared with the blank nanorod-loaded scaffold due to the inhibition of caspase-1. The NA3 nanorod-loaded and blank nanorod-loaded polymeric scaffolds reduced the ASC speck formation compared with the positive control group (p<0.01). When the number of ASC specks per live cell was analyzed by confocal microscopy, a 2-fold reduction in ASC speck formation was observed in iBMDMs treated with NA3 nanorod-loaded polymeric scaffold compared with blank nanorod-loaded polymeric scaffold (FIG. 5h).

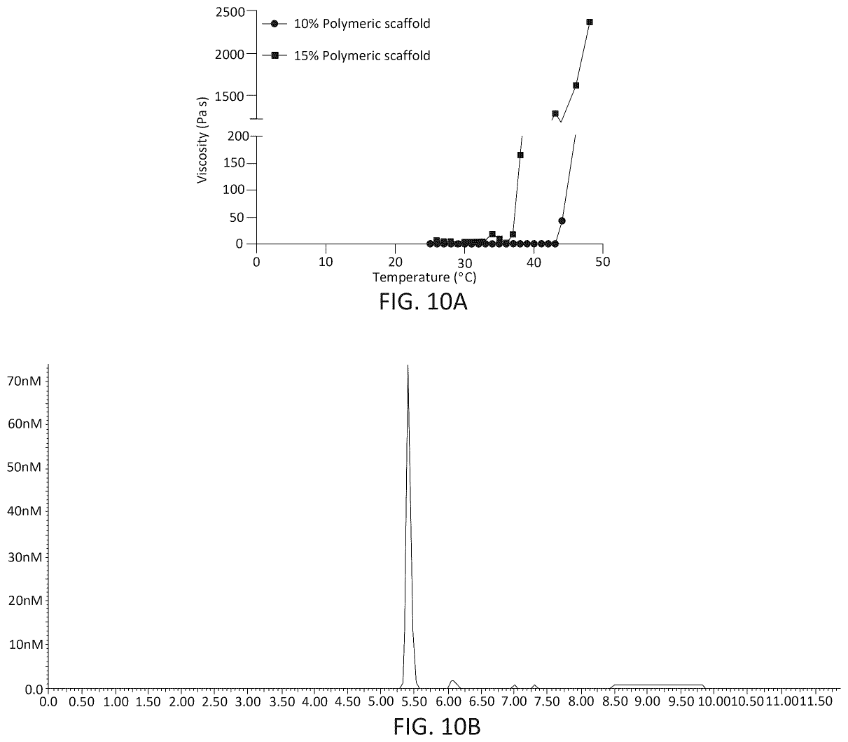

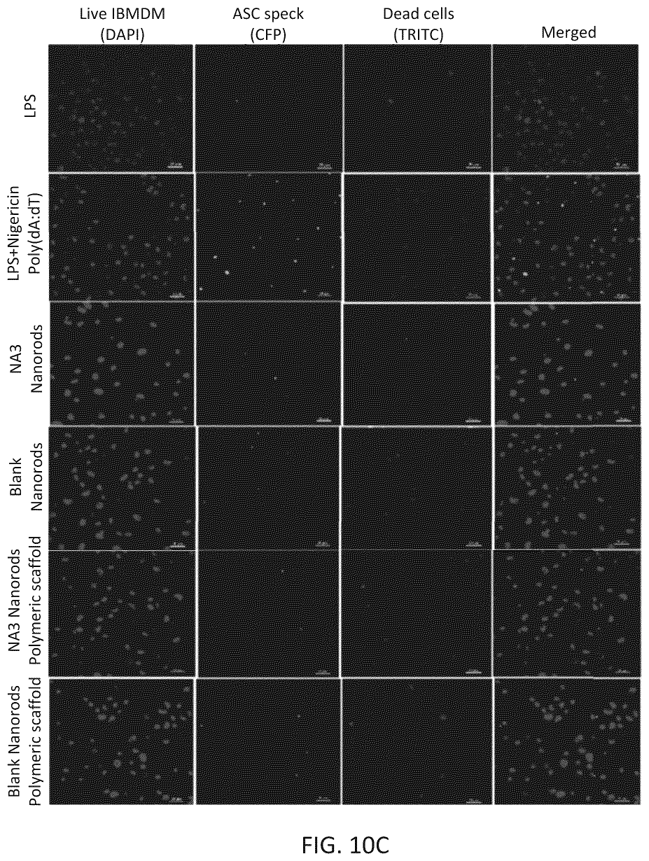

The development of topical spray was instigated to support ease of administration and patient compliance. However, a reversible shear thinning, and solid-like behavior is desirable for quick administration through the spray nozzle (85). Poloxamer 407 aqueous solution is widely used as a fluid that encompasses temperature-dependent yield stress, the ability to reduce viscosity on the application of shear stress and forms a weak gel when in contact with a surface (84). Moreover, the anti-fouling effect and anti-protein adsorption make it suitable for topical spray (86). The viscosity at a sol-gel temperature of Poloxamer 407 was 12.09 and 165 Pa·s at 10% w/v and 15% w/v, respectively (FIG. 10a). However, the higher viscosity of the gel reduces the penetration ability of the nanoparticles within the skin when applied topically. For instance, an increase in viscosity of cellulose from 16.3±1.05 Pa·s to 40.4±3.11 Pa·s reduced penetration of Sulphadiazene sodium by 1.66 folds (87). Therefore, an optimal 10% w/v Poloxamer 407 concentration was used to develop a sprayable scaffold. The mucin provided an additive effect that formed a mesh-like network above 1.875 mg/ml concentration (88). Poloxamer 407 and mucin (as an anti-inflammatory thickening agent) were utilized to develop a sprayable scaffold. An increase in shear rate caused decreased viscosity of the sprayable NA3 nanorod polymeric scaffold, eliciting shear thinning behavior when passed through the spray bottle nozzle (FIG. 5i). The NA3 nanorod scaffold showed sol-gel transition at 43° C. with an increase in viscosity from 2.41 Pa·s (43° C.) to 361.4 Pa·s (49° C.). As per the previous reports, the storage/elastic modulus (G′) and loss/viscous modulus (G″) increased rapidly at the sol-gel transition, eliciting the thermogelling nature of the scaffold (FIG. 5i).

In vivo efficacy of sprayable NA3 nanorod-loaded polymeric scaffold in imiquimod (IMQ)-induced psoriasis-like Balb/c mice.

As per the literature, the 2D and 3D in vitro human cell-based models have been developed using human keratinocytes co-cultured with T-cells/macrophages and inflammatory cytokines aggravating keratinocyte proliferation (89,90). Certain drawbacks with in vitro models include morphological dysfunction of necrosis, spongiosis, and parakeratosis even before losing keratinocyte integrity (91), improper selection of culture media mimicking the fluid volume in the psoriatic skin tissue, dose extrapolation, loss of disease phenotype during cell expansion (90), lack of reproducibility (92) and scarcity of the source of psoriatic lesional skin (90), inability to estimate the vascular inflammation and hypervascularization (90), and inability to predict the systemic effect. On the contrary, the in-vivo model exactly illustrates the involvement of inflammatory mediators, the communication between innate and adaptive immune systems, the role of resident cells, and the evaluation of new therapies (93). Therefore, the efficacy of NA3 nanorods and blank nanorod-loaded polymeric scaffold were investigated in the mice psoriasis model.

Animal models should be selected based on species that develop similar diseases, augmentation of key molecule or cell type, and/or inhibition of target molecule or cell type (93). Therefore, the appropriate animal model for the present study should include an aggravated inflammasome response causing chronic psoriasis-like symptoms which is observed in IMQ-induced or IL-23 induced psoriasis like mice model. IMQ stimulates the activation of NLRP3 and AIM2 inflammasomes through TLR 7/8 receptors (94). Similarly, IL-36 and Keratin-14 (K14) promoters cause psoriasis-like symptoms by activation of NLRP3 inflammasome and IL-17/IL23 axis (95). However, an IMQ-induced psoriasis-like mice model was used in the current study considering its acceptability, case of psoriasis induction, and cost (96). Furthermore, IMQ-induced psoriasis-like models closely resemble human psoriasis including infiltration of both innate and adaptive immune cells (Th17 and Th22 responsible for IL-17A/IL-23 axis activation), dendritic cells, macrophages, keratinocytes leading to parakeratosis, acanthosis, rete-ridges, and vascular hyperproliferation, and hyperplasia (90). Therefore, the difference in the efficacy of blank nanorods, NA3 nanorods treated polymeric scaffold versus IMQ-treated and Vaseline (positive and negative control) were more pronounced in the IMQ-induced psoriasis-like model.

Furthermore, other in vivo models are associated with several disadvantages that includes dermal infiltration including mast cells, macrophages, and neutrophils without the involvement of cross-talk between innate and adaptive immune cells (T-cells), no-efficacy against cyclosporin (calcineurin inhibitor) in spontaneous mutation model (93), complexity in identification and modification of genes to establish chronic skin inflammation in genetically modified mice psoriasis model (97), variation in the penetrance of the transferred T-cell phenotypes and prioritizing only T-cells in immune cell induced psoriasis-like model (93), unpredictable phenotype and inappropriately developed skin model representing more like atopic dermatitis than psoriasis in IL-23 induced psoriasis model (98) indicate IMQ-induced Balb/c mice psoriasis model as a suitable platform to evaluate anti-psoriatic efficacy.

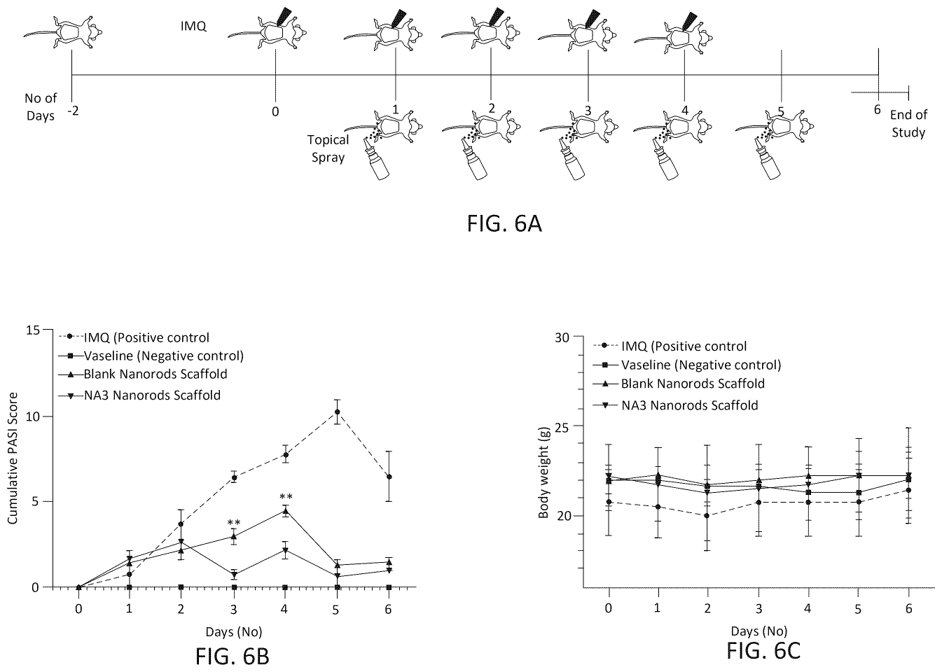

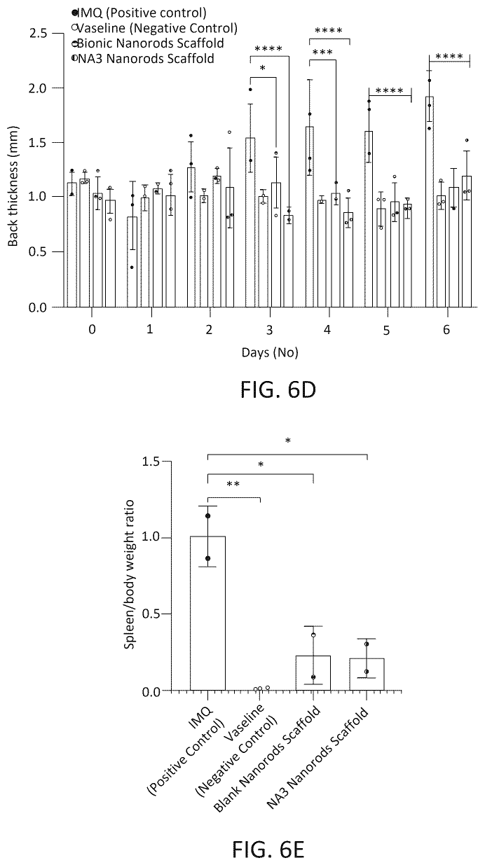

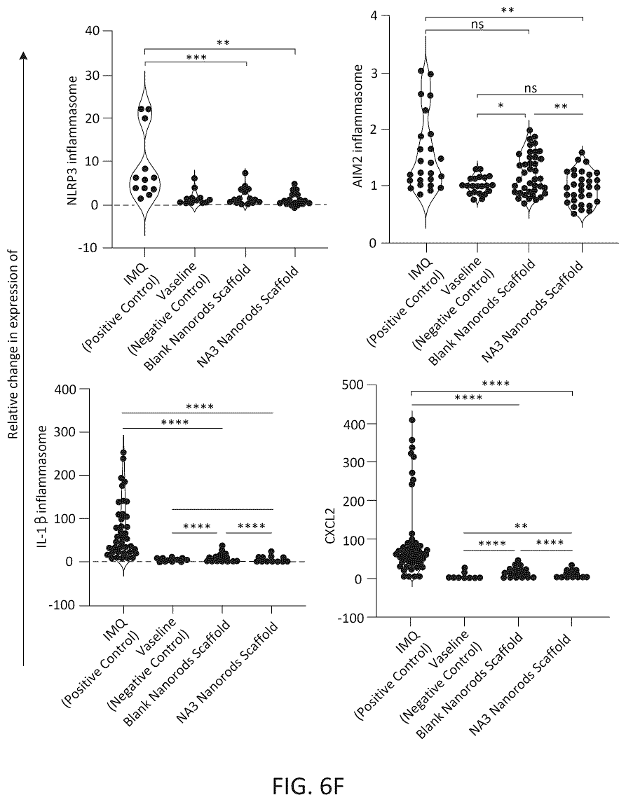

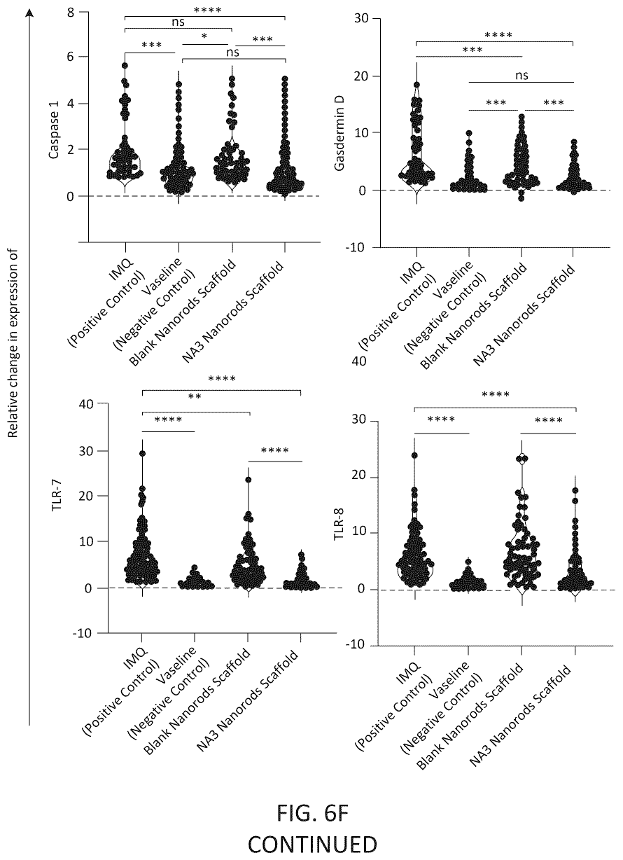

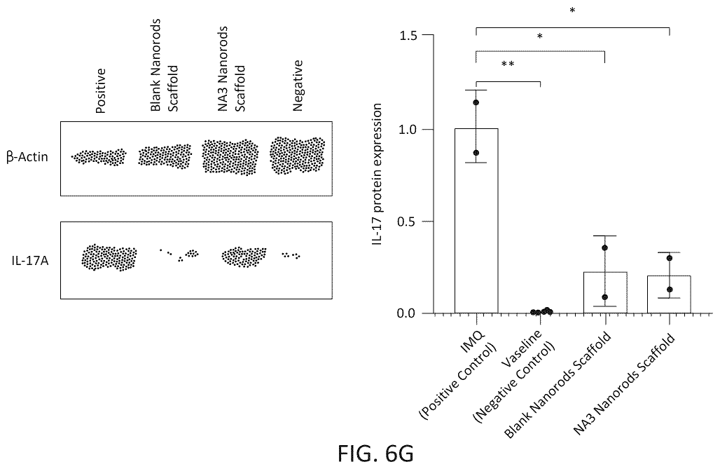

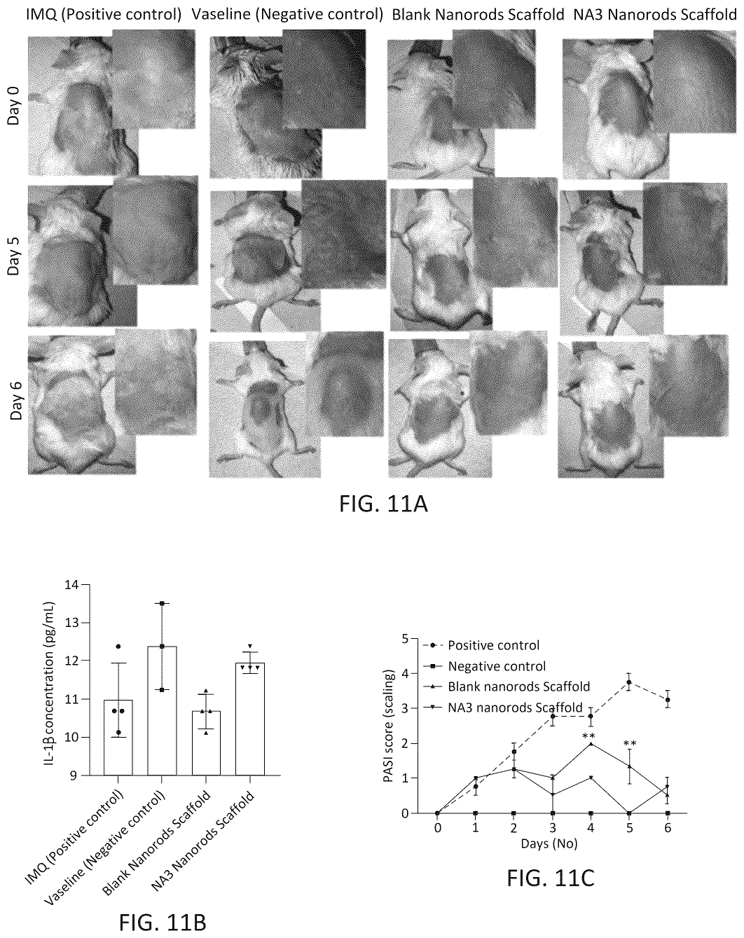

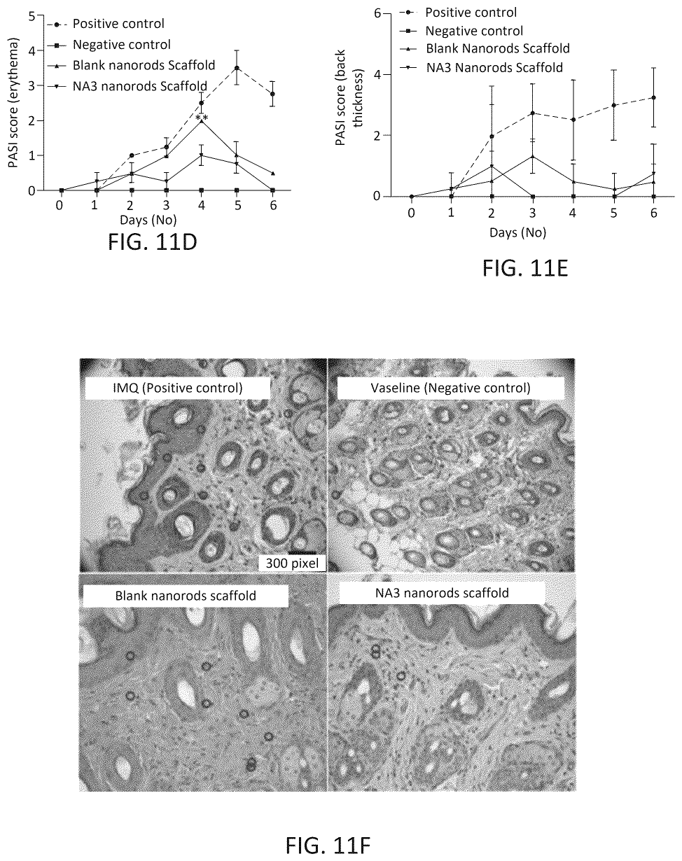

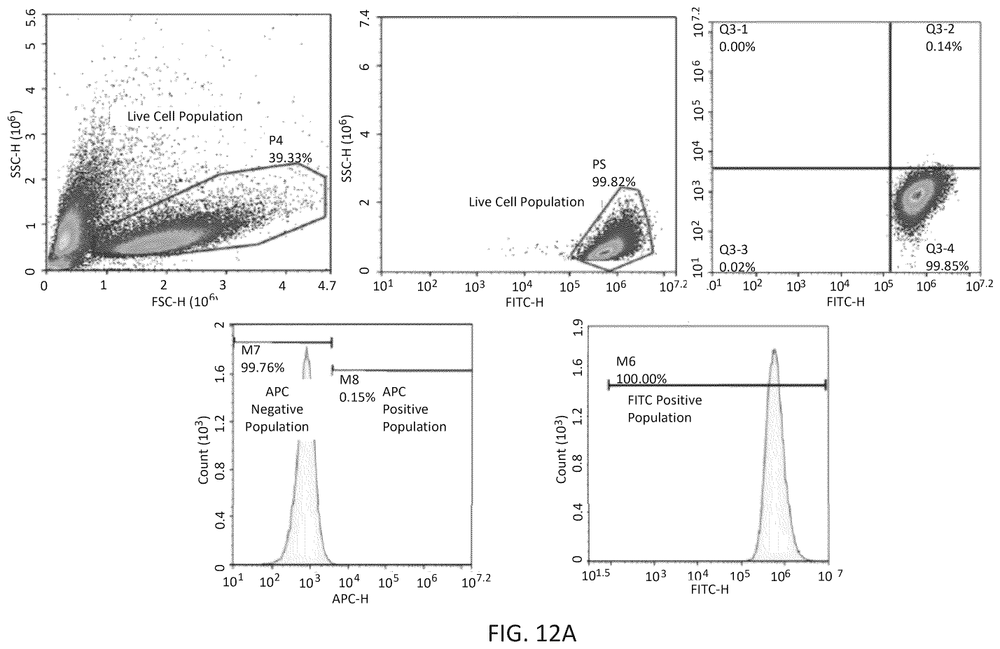

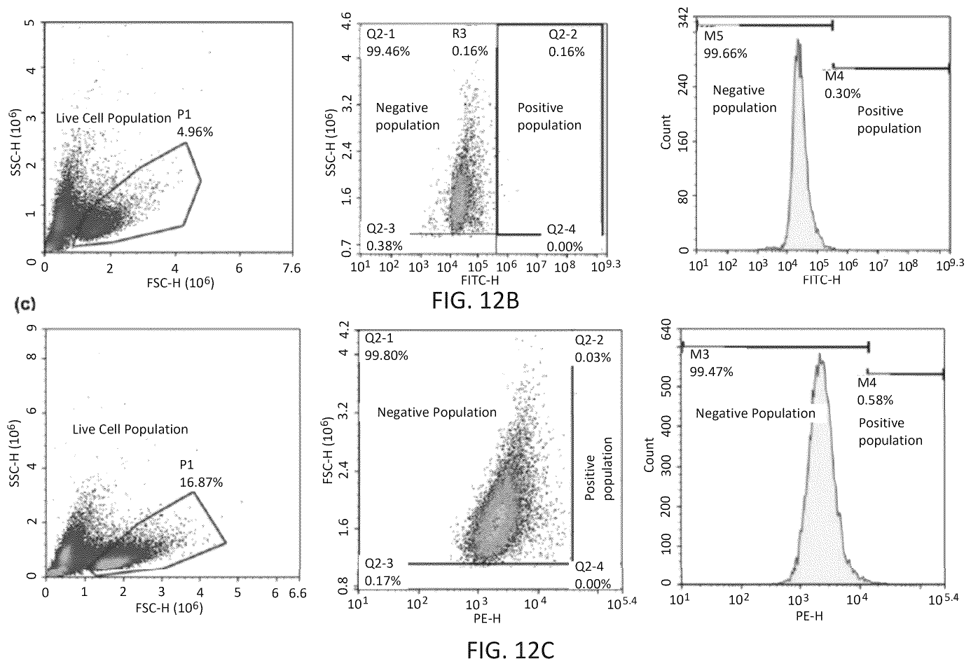

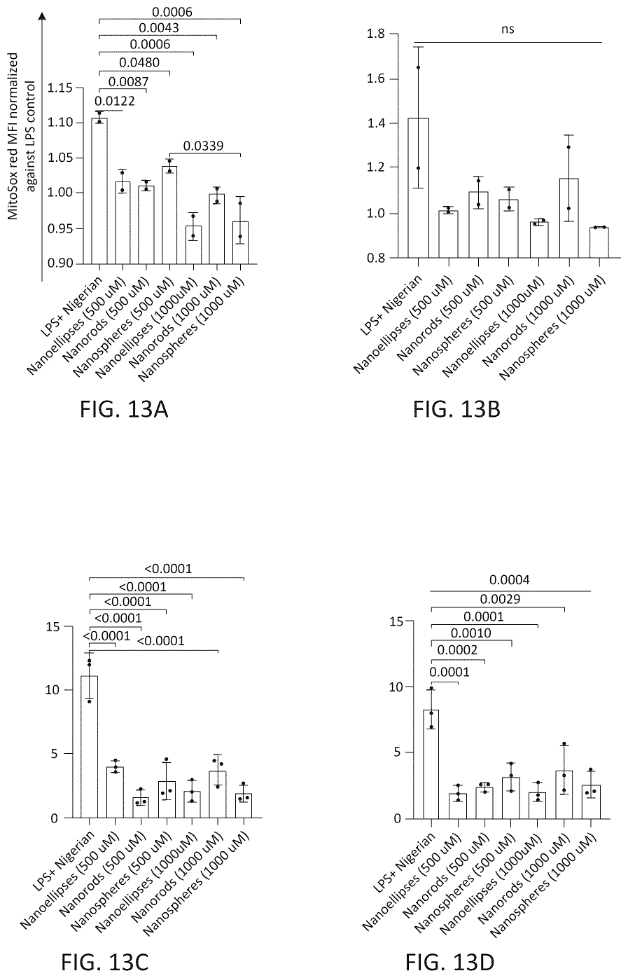

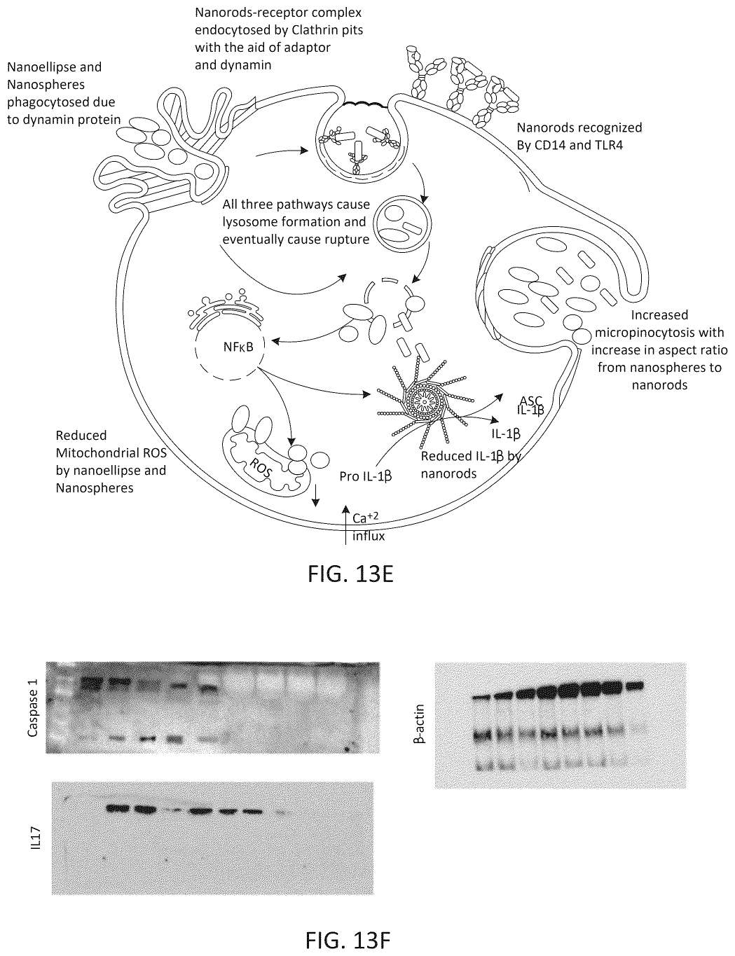

The mice in groups 1 and 2 were treated topically with Vaseline (negative control) and IMQ (62.5 mg/day; positive control) on the shaved back for 5 days. Whereas the mice in groups 3 and 4 were treated with sprayable NA3 loaded polymeric scaffold (equivalent to 800 μM NA3) and sprayable blank nanorod loaded polymeric scaffold (equivalent to 800 μM NA3), respectively post 8h daily treatment with IMQ (62.5 mg/day) until 6 days (FIG. 6a). The mice were observed daily for indication of psoriatic scaling. It was observed that the psoriatic scaling on the mice back skin reduced in the order of IMQ treated (positive control)>blank nanorods scaffold >NA3 nanorods scaffold >Vaseline treated (negative control) (FIG. 11a). The mice in each group were evaluated for erythema, scaling, and thickness using psoriatic area and severity index (PASI) scoring. It was observed that the cumulative PASI score of the mice in group 2 increased to 10.25±1.25 on day 5. The cumulative score on day 5 was 0, 1.33±0.577, and 0.66±0.577 for mice in the negative control, Blank nanorod scaffold, and NA3 nanorod scaffold treated groups, respectively (FIG. 6b). No significant difference was observed in the body weight of the animals (FIG. 6c). The results corroborated previous findings that depicted species and strain-specific effects of IMQ-induced psoriasis on body weight (99).

The back thickness of mice treated with a blank nanorod-loaded polymeric scaffold decreased gradually compared with the NA3 nanorod-loaded polymeric scaffold, indicating higher efficacy of the NA3 nanorod-loaded polymeric scaffold (FIG. 6d). Prolonged inflammation causes an increase in the size and weight of the spleen. Interestingly, the spleen weight of the mice, when normalized against body weight in the NA3 nanorods polymeric scaffold and blank nanorods scaffold treated group, was significantly less (p<0.05) compared with positive control (FIG. 6c). On day 6, the animals were euthanized, and the change in RNA expression levels relative to B-actin of NLRP3, AIM2, IL-1β, Caspase-1, and Gasdermin-D were evaluated from the back skin to elucidate the effect of NA3 nanorod/blank nanorod polymeric scaffold on NLRP3 and AIM2 inflammasomes. The results of qPCR corroborated with the in-vitro efficacy studies. Both NA3 nanorods polymeric scaffold and blank nanorods polymeric scaffold reduced the levels of caspase-1 and NLRP3 inflammasome, resulting in decreased IL-1βcytokine and gasdermin-D release compared with the positive control (FIG. 6f). The reduced inflammatory cytokine also reduced the T-cell recruiting CXCL2 chemokine levels. Although the nanorods were internalized using CD14 receptor, IMQ is endocytosed through TLR7 and TLR8 which enhances the Th17 cell recruitment and increased IL-17A cytokine (100). The TLR 7 and 8 are also increased in presence of single stranded viral RNA and small synthetic compounds (101).

The effect of the blank nanorods/NA3 loaded nanorods scaffold on the TLR7 and TLR8 was studied. The upregulation of TLR7 was alleviated by 1.4- and 4.3-fold in blank nanorods polymeric scaffold and NA3 nanorods polymeric scaffold treated group, respectively compared with IMQ treated group (FIG. 6f). Additionally, the NA3 nanorods scaffold could alleviate the TLR8 by 1.8-fold compared with IMQ treated group (FIG. 6f). The enhanced efficacy of NA3 nanorods polymeric scaffold compared with blank nanorods polymeric scaffold was because of NA3 on AIM2 inflammasome and active caspase-1 where the blank nanorods lacked efficacy compared with positive control (FIG. 6f). Therefore, the RNA expression levels of AIM2 (p<0.01), IL-1β(p<0.0001), Caspase-1 (p<0.001), CXCL2 (p<0.0001), Gasdermin-D (p<0.001), and TLR 7 and 8 (p<0.0001) were significantly reduced in NA3 nanorods polymeric scaffold compared with blank nanorod polymeric scaffold treated mice skin. Moreover, the IL-17A plays a central role in psoriatic inflammation, keratinocyte proliferation, hyperplasia, and micro-abscess formation providing a positive feedback loop in exacerbating immune response (102-104). The Th 17 cells in the psoriatic skin secrete IL-17A which attracts other inflammatory cells including the T-cells, natural killer cells, neutrophils, and mast cells (103). The effect of NA3 nanorods on IL-17A was observed, where IL-17A protein expression from the mice back skin treated with blank nanorods and NA3 nanorods was significantly reduced (p<0.05) compared with IMQ treated mice back skin (positive control) when analyzed by western blot (FIG. 6g). Therefore, it was hypothesized that targeting NLRP3 inflammasomes in macrophages (innate immune cells) serves as negative feedback loop to the Th17 cells reducing the TLR7, TLR8, and IL-17A levels. As expected, localized delivery led to no significant difference in the inflammasome activity systemically when confirmed with IL-1β levels in plasma samples (FIG. 11b).

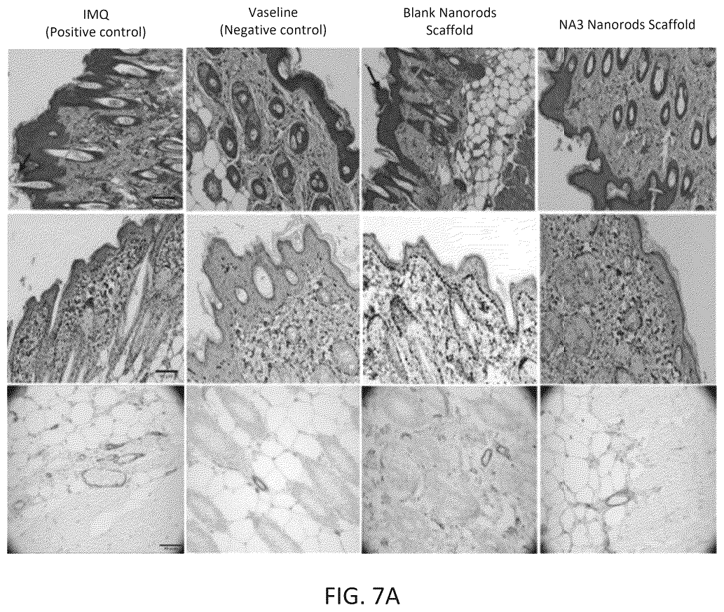

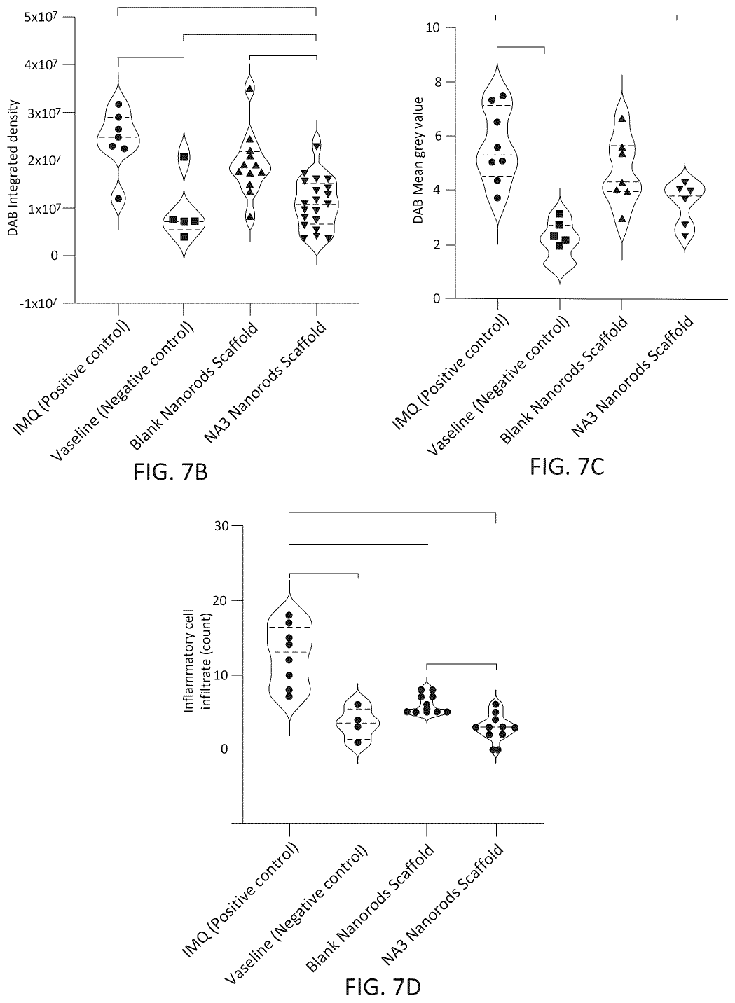

Psoriasis is a dermatological disorder involving epidermal hyperplasia and erythema (105). The commencement of psoriasis includes edema in blood vessels and infiltration of neutrophils and lymphocytes (4). Furthermore, it leads to parakeratosis due to incomplete maturation of keratinocyte retention of nuclei and release of extracellular lipids responsible for adhesion. Therefore, the stratum corneum becomes flaky. It involves the thickening of the epidermis, leading to acanthosis called Rete ridges. The severe form leads to hypogranulosis and migration of neutrophils in the intracorneal region into the stratum corneum called ‘Munro abscess’ and similar accumulation in the epidermis called ‘pustule of Kogoj’ (106,107). The positive control (IMQ treated) mice depicted the characteristic features of psoriatic skin as per the H&E transverse section (FIG. 7a). On the contrary, faded rete ridges, inflammatory infiltration, and capillary inflammation were evident in blank nanorod-loaded polymeric scaffold-treated mice. The epidermal thickness and appearance were similar to negative control in NA3 nanorod-loaded polymeric scaffold-treated mice except for the presence of few inflammatory infiltrates (FIG. 7a).

The mitotic cell cycle phase is higher in psoriatic skin (108). The Ki-67 are nuclear proteins that are increased in rapidly proliferating cells (109). The immunohistochemical analysis of the 3,3′-Diaminobenzidine (DAB) antigen Keil 67 (Ki67) stained samples corroborated with the histopathological analysis. The DAB integrated density of IHC images was 1.8-folds higher in blank nanorods polymeric scaffold treated mice compared with NA3 nanorods polymeric scaffold. Therefore, the IHC analysis further uncovered the synergistic efficacy of NA3, nanorods, and mucin embedded in the polymeric scaffold (FIG. 7a-b). The H&E images and anti-CD31 DAB-stained back skin transverse sections were analyzed microscopically to confirm the vascular hyperproliferation (FIGS. 7a and c) and the extent of inflammatory cell infiltration (FIGS. 7d and 11f). It was observed that the inflammatory cell infiltrates were 2.1-fold and 4.1-fold lower in blank nanorods polymeric scaffold and NA3 nanorods polymeric scaffold treated mice, respectively when compared with the mice treated with IMQ (FIG. 7d). Moreover, the mean grey value of DAB stained anti-CD31 antibody was reduced by 1.6-folds in NA3 nanorods polymeric scaffold treated mice compared with IMQ treated mice (FIG. 7c). To conclude, despite the reduction in the NLRP3 inflammasome, the blank nanorod-loaded polymeric scaffold delayed the reduction in keratinocyte proliferation compared with the NA3 nanorod-loaded polymeric scaffold.

### DISCUSSION

Existing anti-psoriatic therapy majorly consists of vitamin D3, retinoids, corticosteroids, calcineurin inhibitors, biologics, and phototherapy. Systemic, oral, and phototherapy are indicated for patients eliciting failure to first-line topical therapy, and in case of moderate-to-severe psoriasis with a PASI area of >10% (110). Phototherapy involves frequent physician visits decreasing patient compliance. Moreover, it could increase the risk of patient skin cancer and sensitivity to UV light. An increased susceptibility to secondary infections was observed due to systemic immunosuppression (111,112). This leaves the topical localized anti-psoriatic therapy as a suitable treatment approach.

The topical therapy has been majorly explored to inhibit proliferation in T-cells and keratinocytes. The potent or super-potent corticosteroids that inhibit cell proliferation were considered suitable due to their efficacy (113). However, corticosteroids increased the risk of adverse events including atrophy, striae, telangactiaceas, purpura, iatrogenic Cushing syndrome, corticosteroid-related adison crises, growth retardation, mineralocorticoid effect (114,115), psoriatic relapse, and corticosteroid withdrawal symptoms (116) decreasing quality of life in psoriasis patients. Therefore, steroid-sparing agents were highly recommended and promoted for anti-psoriatic effects even if they would elicit delayed but consistent effects (113). Other topical treatment regimens include vitamin D3 derivatives and retinoids targeting keratinocyte differentiation and proliferation (13,117). Only, 14-50% clinical efficacy was obtained with vitamin D3 derivative or tazarotene (retinoid) monotherapy (113) due to their limitations including pH and UV-light sensitivity (Vitamin D3) (113), and erythema, pruritis, and burning associated with tazoretene (13). Another approach includes T-cell targeted topical therapy including Calcineurin inhibitors. Various antibodies were developed with advances in the immunopathology of psoriasis. For instance, bimekizumab (IL-17A and IL-17F blocker) elicited clinical efficacy in 91% of psoriatic patients (118).

The development of antibodies against various receptors and cytokines would lead to an increased cost and unavoidable systemic immunomodulation. Given the drawbacks of existing therapies, there was an urgent need for a safe, efficacious, and cost-effective anti-psoriatic treatment. The majority of the existing drugs fail to target the inflammatory macrophages within psoriatic skin. The NA3 nanorod polymeric scaffolds decreased the IL-1β levels released by macrophages in psoriatic skin which would prevent further activation of Th17 keratinocyte proliferation (81,119). Current work was directed toward the development of novel macrophage-targeted anti-psoriatic therapy which may elicit delayed yet consistent response. To avoid the repeatability as per the 3R principle, considering the adverse effects of existing therapies, and the novelty of the developed NA3 nanorods polymeric scaffold, the blank nanorods polymeric scaffold and NA3 nanorods polymeric scaffold were compared with IMQ treated mice (positive control) to evaluate their ability to improve psoriasis in mice without causing systemic effects.

The non-spherical LNPs were successfully developed, varying the molar ratio of PD and DSPE-PEG 2000 (carboxylic acid) from 2:1 to 10:1 by self-assembled thin-film hydration method. The morphology and stability of the nano-spheres/ellipses/rods were confirmed by transmission electron microscopy (TEM) and dynamic light scattering, respectively. The pathogen-like non-spherical trojan horse LNPs were internalized by macropinocytosis and clathrin-mediated endocytosis. The nanorods were internalized using CD14 while, the nanoellipses were internalized through TLR-4 receptors, respectively. The trojan-horse nanorods attenuated the NLRP3 inflammasomes due to the incorporation of PD. Moreover, it reduced ASC speck, lysosomal rupture, restrained calcium influx, and mitochondrial ROS formation. Developing a sprayable polymeric scaffold for delivering NA3-loaded nanorods elicited case of administration, a simple manufacturing process, and synergistic inhibition of NLRP3 and AIM2 inflammasomes formation along with caspase-1 inhibition. The enhanced efficacy of the NA3 nanorod polymeric scaffold could be attributed to the combination effect of nanorods, NA3, and mucin. The evaluation of sprayable NA3-loaded nanorods in IMQ-induced psoriasis mice model depicted reduced psoriasis as confirmed by qPCR and immunohistopathology. Moreover, the reduced proliferation marker (Ki-67), vascularization, and inflammatory infiltrates confirmed the prodigious anti-psoriatic treatment potential of sprayable NA3-loaded nanorods polymeric scaffold.

To conclude, non-spherical trojan horse nanorods incorporated within a sprayable polymeric scaffold were a novel platform delivery system with anti-inflammatory and anti-psoriatic properties. The sprayable NA3 nanorods-loaded polymeric scaffold paved the way for efficacious and localized delivery against psoriasis, eliminating drawbacks with existing therapies, including systemic immunosuppression and poor skin penetration. This approach could potentially revolutionize psoriasis therapy, eliciting benefits including case of administration, tolerability, and scale-up. The novel prospects of the platform technology can be well explored for other inflammatory dermatological disorders with clinical translation.

### CONCLUSIONS

Inspired by non-spherical pathogens in nature and their ability to target macrophages, anti-inflammatory, non-spherical lipid nanoparticles (LNPs) were synthesized using an anti-inflammatory lipid (pyridoxine dipalmitate) as a trojan horse. The effect of different LNP shapes on the inhibition potential of NLRP3 inflammasomes was investigated. In vitro studies demonstrated that nanoellipses and nanorods were internalized by macropinocytosis and clathrin-mediated endocytosis. In contrast, the nanospheres were phagocytosed by macrophages. The nanorods inhibited NLRP3 inflammasomes by 3.8- and 4.5-fold compared with nanoellipses and nanospheres. Nanorods reduced apoptosis-associated speck-like protein and lysosomal rupture, restrained calcium influx, and mitochondrial reactive oxygen species (ROS) generation. Incorporation of the dual NLRP3 and AIM2 inhibitor (NA3) within nanorods elicited synergistic inhibition with 21.5- and 59-fold reduction in Interleukin-1B (IL-1β) levels compared with nanorods and free NA3, respectively alongside caspase-1 inhibition. The NA3 nanorods were transformed into a sprayable technology using poloxamer 407 (thermogelling agent) and mucin (thickening agent) for localized application. The NA3 nanorod polymeric scaffold simultaneously and effectively inhibited the RNA levels of NLRP3, AIM2, caspase-1, chemokine (C—X—C motif) ligand-2 (CXCL2), gasdermin-D, IL-1β, toll-like receptor (TLR) 7 and 8, and IL-17A by 6.4-, 1.6-, 2.0-, 13.0-, 4.2-, 24.4-, 4.3-, and 1.82-fold, respectively in psoriatic skin when compared with Imiquimod (IMQ) positive control group in an in vivo psoriasis-like mice model. The NA3 nanorod polymeric scaffold reduced the cumulative psoriatic area severity index (PASI) score for thickness, erythema, and scaling by 15.4-fold compared with the IMQ positive control group, respectively without causing an effect on plasma IL-1βlevel. In summary, the sprayable NA3 nanorod-loaded polymeric scaffold served as a simple platform for localized anti-psoriatic delivery, elicited synergy by targeting NLRP3 and AIM2 dual inhibition, and avoided harmful systemic side effects.

### BIBLIOGRAPHY

(1) Armstrong, A. W.; Read, C. Pathophysiology, Clinical Presentation, and Treatment of Psoriasis: A Review. JAMA-J. Am. Med. Assoc. 2020, 323 (19), 1945-1960. https://doi.org/10.1001/jama.2020.4006.
(2) Global Report on Psoriasis; 2016.
(3) Rendon, A.; Schäkel, K. Psoriasis Pathogenesis and Treatment. Int. J. Mol. Sci. 2019, 20 (6), 1-28. https://doi.org/10.3390/ijms20061475.
(4) Yamanaka, K.; Yamamoto, O.; Honda, T. Pathophysiology of Psoriasis: A Review. J. Dermatol. 2021, 48 (6), 722-731. https://doi.org/10.1111/1346-8138.15913.
(5) Zhou, X.; Chen, Y.; Cui, L.; Shi, Y.; Guo, C. Advances in the Pathogenesis of Psoriasis: From Keratinocyte Perspective. Cell Death Dis. 2022, 13 (1). https://doi.org/10.1038/s41419-022-04523-3.
(6) Verma, D.; Fekri, S. Z.; Sigurdardottir, G.; Bivik Eding, C.; Sandin, C.; Enerback, C. Enhanced Inflammasome Activity in Patients with Psoriasis Promotes Systemic Inflammation. J. Invest. Dermatol. 2021, 141 (3), 586-595.e5. https://doi.org/10.1016/j.jid.2020.07.012.
(7) Ciążyńska, M.; Olejniczak-Staruch, 1.; Sobolewska-Sztychny, D.; Narbutt, J.; Skibińska, M.; Lesiak, A. The Role of NLRP1, NLRP3, and AIM2 Inflammasomes in Psoriasis: Review. Int. J. Mol. Sci. 2021, 22 (11), 1-11. https://doi.org/10.3390/ijms22115898.
(8) Wang, H.; Syrovets, T.; Kess, D.; Büchele, B.; Hainzl, H.; Lunov, O.; Weiss, J. M.; Scharffetter-Kochanek, K.; Simmet, T. Targeting NF-κB with a Natural Triterpenoid Alleviates Skin Inflammation in a Mouse Model of Psoriasis. J. Immunol. 2009, 183 (7), 4755-4763. https://doi.org/10.4049/jimmunol.0900521.
(9) Nowowiejska, J.; Baran, A.; Hermanowicz, J. M.; Pryczynicz, A.; Sieklucka, B.; Pawlak, D.; Flisiak, I. Gasdermin D (GSDMD) Is Upregulated in Psoriatic Skin-A New Potential Link in the Pathogenesis of Psoriasis. Int. J. Mol. Sci. 2023, 24 (17). https://doi.org/10.3390/ijms241713047.
(10) Stanescu, A. M. A.; Simionescu, A. A.; Diaconu, C. C. Oral Vitamin D Therapy in Patients with Psoriasis. Nutrients 2021, 13 (1), 1-12. 34 https://doi.org/10.3390/nu13010163.
(11) Sadeghinia, A.; Daneshpazhooh, M. Immunosuppressive Drugs for Patients with Psoriasis during the COVID-19 Pandemic Era. A Review. Dermatol. Ther. 2021, 34 (1), 1-9. https://doi.org/10.1111/dth.14498.
(12) Jiang, Y.; Chen, Y.; Yu, Q.; Shi, Y. Biologic and Small-Molecule Therapies for Moderate-to-Severe Psoriasis: Focus on Psoriasis Comorbidities. BioDrugs 2023, 37 (1), 35-55. https://doi.org/10.1007/s40259-022-00569-z.
(13) Timis, T. L.; Florian, I. A.; Vesa, S. C.; Mitrea, D. R.; Orasan, R. I. An Updated Guide in the Management of Psoriasis for Every Practitioner; 2021; Vol. 75. https://doi.org/10.1111/ijcp.14290.
(14) Szymański, Ł.; Skopek, R.; Palusińska, M.; Schenk, T.; Stengel, S.; Lewicki, S.; Kraj, L.; Kamiński, P.; Zelent, A. Retinoic Acid and Its Derivatives in Skin. Cells 2020, 9 (12), 1-14. https://doi.org/10.3390/cells9122660.
(15) van de Kerkhof, P. C. M.; de Gruijl, F. R. Phototherapy in the Perspective of the Chronicity of Psoriasis. J. Eur. Acad. Dermatology Venereol. 2020, 34 (5), 926-931. https://doi.org/10.1111/jdv.16245.
(16) Bos, J. D.; Spuls, P. I. Topical Treatments in Psoriasis: Today and Tomorrow. Clin. Dermatol. 2008, 26 (5), 432-437. https://doi.org/10.1016/j.clindermatol.2007.10.025.
(17) Vanaja, S. K.; Rathinam, V. A. K.; Fitzgerald, K. A. Mechanisms of Inflammasome Activation: Recent Advances and Novel Insights. Trends Cell Biol. 2015, 25 (5), 308-315. https://doi.org/10.1016/j. tcb.2014.12.009.
(18) Nandi, D.; Farid, N. S. S.; Karuppiah, H. A. R.; Kulkarni, A. Imaging Approaches to Monitor Inflammasome Activation. J. Mol. Biol. 2022, 434 (4), 167251. https://doi.org/10.1016/j. jmb.2021.167251.
(19) Tan, R. zhi; Zhong, X.; Han, R. yue; Xie, K. huan; Jia, J.; Yang, Y.; Cheng, M.; Yang, C. yan; Lan, H. yao; Wang, L. Macrophages Mediate Psoriasis via Mincle-Dependent Mechanism in Mice. Cell Death Discov. 2023, 9 (1). https://doi.org/10.1038/s41420-023-01444-8.
(20) He, X.; Shen, C.; Lu, Q.; Li, J.; Wei, Y.; He, L.; Bai, R.; Zheng, J.; Luan, N.; Zhang, Z.; Rong, M.; Lai, R. Prokineticin 2 Plays a Pivotal Role in Psoriasis. EBioMedicine 35 2016, 13, 248-261. https://doi.org/10.1016/j.ebiom.2016.10.022.
(21) Tu, Q.; Yu, X.; Xie, W.; Luo, Y.; Tang, H.; Chen, K.; Ruan, Y.; Li, Y.; Zhou, J.; Yin, Y.; Chen, D.; Song, Z. Prokineticin 2 Promotes Macrophages-Mediated Antibacterial Host Defense against Bacterial Pneumonia. Int. J. Infect. Dis. 2022, 125, 103-113. https://doi.org/10.1016/j.ijid.2022.10.003.
(22) Davies, L. C.; Rosas, M.; Jenkins, S. J.; Liao, C. Te; Scurr, M. J.; Brombacher, F.; Fraser, D. J.; Allen, J. E.; Jones, S. A.; Taylor, P. R. Distinct Bone Marrow-Derived and Tissue-Resident Macrophage Lineages Proliferate at Key Stages during Inflammation. Nat. Commun. 2013, 4 (0). https://doi.org/10.1038/ncomms2877.
(23) Yang, L.; Fu, J.; Han, X.; Zhang, C.; Xia, L.; Zhu, R.; Huang, S.; Xiao, W.; Yu, H.; Gao, Y.; Liang, Q.; Li, W.; Zhou, Y. Hsa_circ_0004287 Inhibits Macrophage-Mediated Inflammation in an N6-Methyladenosine-Dependent Manner in Atopic Dermatitis and Psoriasis. J. Allergy Clin. Immunol. 2022, 149 (6), 2021-2033. https://doi.org/10.1016/j.jaci.2021.11.024.
(24) Zahid, A.; Li, B.; Kombe, A. J. K.; Jin, T.; Tao, J. Pharmacological Inhibitors of the Nlrp3 Inflammasome. Front. Immunol. 2019, 10 (OCT), 1-10. https://doi.org/10.3389/fimmu.2019.02538.
(25) Tuoda, M. A. T.; Tu, M.; Ana, A. T. A.; Att, T.; Hati, H. A. N. Lipid Nanoparticles Comprising Lipophilic Anti-Inflammatory Agents and Methods of Use Thereof. 2017, 1.
(26) Dipika, N.; Maharshi, D.; James, F. I.; Ankit, P.; Ashish, K. Nanoparticle-Mediated Co-Delivery of Inflammasome Inhibitors Provide Protection against Sepsis. Nanoscale 2024. https://doi.org/10.1039/D3NR05570A.
(27) Zamansky, M.; Yariv, D.; Feinshtein, V.; Ben-Shabat, S.; Sintov, A. C. Cannabidiol-Loaded Lipid-Stabilized Nanoparticles Alleviate Psoriasis Severity in Mice: A New Approach for Improved Topical Drug Delivery. Molecules 2023, 28 (19), 1-16. https://doi.org/10.3390/molecules28196907.
(28) Rahmanian-Devin, P.; Askari, V. R.; Sanei-Far, Z.; Baradaran Rahimi, V.; Kamali, H.; Jaafari, M. R.; Golmohammadzadeh, S. Preparation and Characterization of Solid Lipid Nanoparticles Encapsulated Noscapine and Evaluation of Its Protective Effects 36 against Imiquimod-Induced Psoriasis-like Skin Lesions. Biomed. Pharmacother. 2023, 168 (June), 115823. https://doi.org/10.1016/j.biopha.2023.115823.
(29) Tenchov, R.; Bird, R.; Curtze, A. E.; Zhou, Q. Lipid Nanoparticles from Liposomes to MRNA Vaccine Delivery, a Landscape of Research Diversity and Advancement. ACS Nano 2021, 15 (11), 16982-17015. https://doi.org/10.1021/acsnano.1c04996.
(30) Hou, X.; Zaks, T.; Langer, R.; Dong, Y. Lipid Nanoparticles for MRNA Delivery. Nat. Rev. Mater. 2021, 6 (12), 1078-1094. https://doi.org/10.1038/s41578-021-00358-0.
(31) Maharshi, D.; James, F. I.; Anujan, R.; Kulkarni, As. Protein Corona Formation on Lipid Nanoparticles Negatively Affects the NLRP3 Inflammasome Activation. Bioconjug. Chem. 2023, 34 (10), 1766-1779.
(32) Forster, J.; Nandi, D.; Kulkarni, A. MRNA-Carrying Lipid Nanoparticles That Induce Lysosomal Rupture Activate NLRP3 Inflammasome and Reduce MRNA Transfection Efficiency. Biomater. Sci. 2022, 10 (19), 5566-5582. https://doi.org/10.1039/d2bm00883a.
(33) Li, T.; He, J.; Horvath, G.; Próchnicki, T.; Latz, E.; Takeoka, S. Lysine-Containing Cationic Liposomes Activate the NLRP3 Inflammasome: Effect of a Spacer between the Head Group and the Hydrophobic Moieties of the Lipids. Nanomedicine Nanotechnology, Biol. Med. 2018, 14 (2), 279-288. https://doi.org/10.1016/j.nano.2017.10.011.
(34) Lonez, C.; Bessodes, M.; Scherman, D.; Vandenbranden, M.; Escriou, V.; Ruysschaert, J. M. Cationic Lipid Nanocarriers Activate Toll-like Receptor 2 and NLRP3 Inflammasome Pathways. Nanomedicine Nanotechnology, Biol. Med. 2014, 10 (4), 775-782. https://doi.org/10.1016/j.nano.2013.12.003.
(35) Granot, Y.; Peer, D. Delivering the Right Message: Challenges and Opportunities in Lipid Nanoparticles-Mediated Modified MRNA Therapeuti—s-An Innate Immune System Standpoint. Semin. Immunol. 2017, 34 (July), 68-77. https://doi.org/10.1016/j.smim.2017.08.015.
(36) Lee, Y.; Jeong, M.; Park, J.; Jung, H.; Lee, H. Immunogenicity of Lipid Nanoparticles and Its Impact on the Efficacy of MRNA Vaccines and Therapeutics. Exp. Mol. Med. 2023, 55 (10), 2085-2096. https://doi.org/10.1038/s12276-023-01086-x. 37
(37) Zhu, X.; Xu, Y.; Wang, J.; Xuc, Z.; Qiu, T.; Chen, J. Loss of NLRP3 Reduces Oxidative Stress and Polarizes Intratumor Macrophages to Attenuate Immune Attack on Endometrial Cancer. Front. Immunol. 2023, 14 (April), 1-10. https://doi.org/10.3389/fimmu.2023.1165602.
(38) Ni, D.; Qing, S.; Ding, H.; Yuc, H.; Yu, D.; Wang, S.; Luo, N.; Su, Z.; Wei, W.; Ma, G. Biomimetically Engineered Demi-Bacteria Potentiate Vaccination against Cancer. Adv. Sci. 2017, 4 (10). https://doi.org/10.1002/advs.201700083.
(39) Doshi, N.; Mitragotri, S. Macrophages Recognize Size and Shape of Their Targets. PLOS One 2010, 5 (4), 1-6. https://doi.org/10.1371/journal.pone.0010051.
(40) Wang, L.; Rho, J. G.; Yang, Y.; Seulbi, L.; Sohui, K.; Kim, K.-M.; Yoon, J.; CHoi, H.; Lee, E.; Kim, S. H.; You, S.; Song, Y.; Oh, Y. S.; Kim, H.; Han, H.-S.; Han, J. H.; Jung, M.; Park, Y. H.; Choi, Y. S.; Han, S.; Lec, J.; Choi, S.; Kim, J.-W.; Park, J. H.; Lec, E. K.; Song, W. K.; Kim, E.; Kim, W. Hyaluronic Acid Nanoparticles as a Topical Agent for Treating Psoriasis. ACS Nano 2022, 16 (12), 20057-20074.
(41) Tsuji, G.; Hashimoto-Hachiya, A.; Yen, V. H.; Takemura, M.; Yumine, A.; Furue, K.; Furue, M.; Nakahara, T. Metformin Inhibits IL-1βSecretion via Impairment of NLRP3 Inflammasome in Keratinocytes: Implications for Preventing the Development of Psoriasis. Cell Death Discov. 2020, 6 (1). https://doi.org/10.1038/s41420-020-0245-8.
(42) Deng, G.; Chen, W.; Wang, P.; Zhan, T.; Zheng, W.; Gu, Z.; Wang, X.; Ji, X.; Sun, Y. Inhibition of NLRP3 Inflammasome-Mediated Pyroptosis in Macrophage by Cycloastragenol Contributes to Amelioration of Imiquimod-Induced Psoriasis-like Skin Inflammation in Mice. Int. Immunopharmacol. 2019, 74 (June), 105682. https://doi.org/10.1016/j.intimp.2019.105682.
(43) Chung, I. C.; Yuan, S. N.; OuYang, C. N.; Hu, S. I.; Lin, H. C.; Huang, K. Y.; Lin, W. N.; Chuang, Y. T.; Chen, Y. J.; Ojcius, D. M.; Chang, Y. S.; Chen, L. C. EFLA 945 Restricts AIM2 Inflammasome Activation by Preventing DNA Entry for Psoriasis Treatment. Cytokine 2020, 127 (November 2019), 154951. https://doi.org/10.1016/j.cyto.2019.154951.
(44) Qiao, P.; Zhang, C.; Yu, J.; Shao, S.; Zhang, J.; Fang, H.; Chen, J.; Luo, Y.; Zhi, D.; Li, Q.; Ma, J.; Fu, M.; Dang, E.; Yin, W.; Wang, G. Quinolinic Acid, a Tryptophan Metabolite of the Skin Microbiota, Negatively Regulates NLRP3 Inflammasome 38 through AhR in Psoriasis. J. Invest. Dermatol. 2022, 142 (8), 2184-2193.e6. https://doi.org/10.1016/j.jid.2022.01.010.
(45) Gaire, B. P.; Lee, C. H.; Kim, W.; Sapkota, A.; Lee, D. Y.; Choi, J. W. Lysophosphatidic Acid Receptor 5 Contributes to Imiquimod-Induced Psoriasis-Like Lesions through NLRP3 Inflammasome Activation in Macrophages. Cells 2020, 9 (8). https://doi.org/10.3390/cells9081753.
(46) Dhar, P.; McAuley, J. The Role of the Cell Surface Mucin MUCI as a Barrier to Infection and Regulator of Inflammation. Front. Cell. Infect. Microbiol. 2019, 9 (APR), 1-8. https://doi.org/10.3389/fcimb.2019.00117.
(47) Ciesielska, A.; Matyjek, M.; Kwiatkowska, K. TLR4 and CD14 Trafficking and Its Influence on LPS-Induced pro-Inflammatory Signaling. Cell. Mol. Life Sci. 2021, 78 (4), 1233-1261. https://doi.org/10.1007/s00018-020-03656-y.
(48) Nejadmansouri, M.; Eskandari, M. H.; Yousefi, G. H.; Riazi, M.; Hosseini, S. M. H. Promising Application of Probiotic Microorganisms as Pickering Emulsions Stabilizers. Sci. Rep. 2023, 13 (1), 1-18. https://doi.org/10.1038/s41598-023-43087-w.
(49) Tang, L.; Zhou, F. Inflammasomes in Common Immune-Related Skin Diseases. Front. Immunol. 2020, 11 (May), 1-15. https://doi.org/10.3389/fimmu.2020.00882.
(50) Zhang, P.; Tsuchiya, K.; Kinoshita, T.; Kushiyama, H.; Suidasari, S.; Hatakeyama, M.; Imura, H.; Kat, N.; Suda, T. Vitamin B6 Prevents IL-1β Protein Production by Inhibiting NLRP3 Inflammasome Activation. J. Biol. Chem. 2016, 291 (47), 24517-24527. https://doi.org/10.1074/jbc.M116.743815.
(51) Paolino, D.; Accolla, M. L.; Cilurzo, F.; Cristiano, M. C.; Cosco, D.; Castelli, F.; Sarpietro, M. G.; Fresta, M.; Celia, C. Interaction between PEG Lipid and DSPE/DSPC Phospholipids: An Insight of PEGylationDegree and Kinetics of de-PEGylation. Colloids Surfaces B Biointerfaces 2017, 155, 266-275. https://doi.org/10.1016/j.colsurfb.2017.04.018.
(52) Higashi, K.; Mibu, F.; Saito, K.; Limwikrant, W.; Yamamoto, K.; Moribe, K. Composition-Dependent Structural Changes and Antitumor Activity of ASC-DP/DSPE-PEG Nanoparticles. Eur. J. Pharm. Sci. 2017, 99, 24-31. https://doi.org/10.1016/j.ejps.2016.11.029. 39
(53) Kowalska, M.; Broniatowski, M.; Mach, M.; Płachta, Ł.; Wydro, P. Effect of Lipopolymer (DSPE-PEG750) on Phospholipid Monolayers and Bilayers Differing in the Structure of the Polar Head Group. J. Mol. Liq. 2021, 344, 117715. https://doi.org/10.1016/j.molliq.2021.117715.
(54) Kowalska, M.; Broniatowski, M.; Mach, M.; Płachta, Ł.; Wydro, P. The Effect of the Polyethylene Glycol Chain Length of a Lipopolymer (DSPE-PEGn) on the Properties of DPPC Monolayers and Bilayers. J. Mol. Liq. 2021, 335, 116529. https://doi.org/10.1016/j.molliq.2021.116529.
(55) Chen, Z.; Higashi, K.; Shigehisa, Y.; Ueda, K.; Yamamoto, K.; Moribe, K. Understanding the Rod-to-Tube Transformation of Self-Assembled Ascorbyl Dipalmitate Lipid Nanoparticles Stabilized with PEGylated Lipids. Nanoscale 2022, 15 (6), 2602-2613. https://doi.org/10.1039/d2nr04987b.
(56) Chen, Z.; Higashi, K.; Shidara, R.; Ueda, K.; Morita, T.; Limwikrant, W.; Yamamoto, K.; Moribe, K. The Nanostructure of Rod-like Ascorbyl Dipalmitate Nanoparticles Stabilized by a Small Amount of DSPE-PEG. Int. J. Pharm. 2021, 602 (April), 120599. https://doi.org/10.1016/j.ijpharm.2021.120599.
(57) Ferreira, S. A.; Correia, A.; Madureira, P.; Vilanova, M.; Gama, F. M. Unraveling the Uptake Mechanisms of Mannan Nanogel in Bone-Marrow-Derived Macrophages. Macromol. Biosci. 2012, 12 (9), 1172-1180. https://doi.org/10.1002/mabi.201200075.
(58) Vasudevan, S. O.; Russo, A. J.; Kumari, P.; Vanaja, S. K.; Rathinam, V. A. A TLR4-Independent Critical Role for CD14 in Intracellular LPS Sensing. Cell Rep. 2022, 39 (5), 110755. https://doi.org/10.1016/j.celrep.2022.110755.
(59) Zanoni, I.; Tan, Y.; Di Gioia, M.; Springstead, J. R.; Kagan, J. C. By Capturing Inflammatory Lipids Released from Dying Cells, the Receptor CD14 Induces Inflammasome-Dependent Phagocyte Hyperactivation. Immunity 2017, 47 (4), 697-709.e3. https://doi.org/10.1016/j.immuni.2017.09.010.
(60) Madej, M. P.; Töpfer, E.; Boraschi, D.; Italiani, P. Different Regulation of Interleukin-1 Production and Activity in Monocytes and Macrophages: Innate Memory as an Endogenous Mechanism of IL-1 Inhibition. Front. Pharmacol. 2017, 8 (JUN), 1-12. https://doi.org/10.3389/fphar.2017.00335. 40
(61) Nandi, D.; Shivrayan, M.; Gao, J.; Krishna, J.; Das, R.; Liu, B.; Thayumanavan, S.; Kulkarni, A. Core Hydrophobicity of Supramolecular Nanoparticles Induces NLRP3 Inflammasome Activation. ACS Appl. Mater. Interfaces 2021, 13 (38), 45300-45314. https://doi.org/10.1021/acsami.lc14082.
(62) Yang, Y.; Wang, H.; Kouadir, M.; Song, H.; Shi, F. Recent Advances in the Mechanisms of NLRP3 Inflammasome Activation and Its Inhibitors. Cell Death Dis. 2019, 10 (2). https://doi.org/10.1038/s41419-019-1413-8.
(63) Makvandi, P.; Zarepour, A.; Zheng, X.; Agarwal, T.; Ghomi, M.; Sartorius, R.; Zare, E. N.; Zarrabi, A.; Wu, A.; Maiti, T. K.; Smith, B. R.; Varma, R. S.; Tay, F. R.; Mattoli, V. Non-Spherical Nanostructures in Nanomedicine: From Noble Metal Nanorods to Transition Metal Dichalcogenide Nanosheets. Appl. Mater. Today 2021, 24, 101107. https://doi.org/10.1016/j.apmt.2021.101107.
(64) Lin, H. C.; Chiang, H. P.; Jiang, W. P.; Lan, Y. H.; Huang, G. J.; Hsieh, M. T.; Kuo, S. C.; Lo, C. L.; Chiang, Y. T. Exploitation of a Rod-Shaped, Acid-Labile Curcumin-Loaded Polymeric Nanogel System in the Treatment of Systemic Inflammation. Mater. Sci. Eng. C 2021, 133 (December 2021), 112597. https://doi.org/10.1016/j.msec.2021.112597.
(65) Blevins, H. M.; Xu, Y.; Biby, S.; Zhang, S. The NLRP3 Inflammasome Pathway: A Review of Mechanisms and Inhibitors for the Treatment of Inflammatory Diseases. Front. Aging Neurosci. 2022, 14 (June), 1-27. https://doi.org/10.3389/fnagi.2022.879021.
(66) Yu, T.-G.; Cha, J. S.; Kim, G.; Sohn, Y.-K.; Yoo, Y.; Kim, U.; Song, J.-J.; Cho, H.-S.; Kim, H.-S. Oligomeric States of ASC Specks Regulate Inflammatory Responses by Inflammasome in the Extracellular Space. Cell death Discov. 2023, 9 (1), 142. https://doi.org/10.1038/s41420-023-01438-6.
(67) Zhao, Y.; Jiang, S.; Zhang, J.; Guan, X. L.; Sun, B. G.; Sun, L. A Virulent Bacillus Cereus Strain from Deep-Sea Cold Seep Induces Pyroptosis in a Manner That Involves NLRP3 Inflammasome, JNK Pathway, and Lysosomal Rupture. Virulence 2021, 12 (1), 1362-1376. https://doi.org/10.1080/21505594.2021.1926649.
(68) Mathur, A.; Kay, C.; Xue, Y.; Pandey, A.; Lee, J.; Jing, W.; Tuipulotu, D. E.; Pilato, J. Lo; Feng, S.; Ngo, C.; Zhao, A.; Shen, C.; Rug, M.; Miosge, L. A.; Atmosukarto, 1. I.; 41 Price, J. D.; Ali, S. A.; Gardiner, E. E.; Robertson, A. A. B.; Awad, M. M.; Lyras, D.; Kaakoush, N. O.; Man, S. M. Clostridium Perfringens Virulence Factors Are Nonredundant Activators of the NLRP 3 Inflammasome. EMBO Rep. 2023, 1-20. https://doi.org/10.15252/embr.202254600.
(69) Antushevich, H. Interplays between Inflammasomes and Viruses, Bacteria (Pathogenic and Probiotic), Yeasts and Parasites. Immunol. Lett. 2020, 228 (September), 1-14. https://doi.org/10.1016/j.imlet.2020.09.004.
(70) Campden, R. I.; Zhang, Y. The Role of Lysosomal Cysteine Cathepsins in NLRP3 Inflammasome Activation. Arch. Biochem. Biophys. 2019, 670 (February), 32-42. https://doi.org/10.1016/j.abb.2019.02.015.
(71) Jing, W.; Lo Pilato, J.; Kay, C.; Man, S. M. Activation Mechanisms of Inflammasomes by Bacterial Toxins. Cell. Microbiol. 2021, 23 (4). https://doi.org/10.1111/cmi.13309.
(72) Mercer, J.; Helenius, A. Virus Entry by Macropinocytosis. Nat. Cell Biol. 2009, 11 (5), 510-520. https://doi.org/10.1038/ncb0509-510.
(73) Feng, X.; Junsheng, Y. Lysosomal Calcium in Neurodegeneration. Messenger (Los Angel) 2017, 5 (1-2), 56-66. https://doi.org/10.1053/j.gastro.2016.08.014.CagY.
(74) Brough, D.; Feuvre, R. A. Le; Wheeler, R. D.; Solovyova, N.; Hilfiker, S.; Rothwell, N. J.; Verkhratsky, A. Ca 2+Stores and Ca 2+ Entry Di Erentially Contribute to the Release of IL-1βand IL-1α from Murine Macrophages. J Immunol 2003, 170 (6), 3029-3036.
(75) Yaron, J. R.; Gangaraju, S.; Rao, M. Y.; Kong, X.; Zhang, L.; Su, F.; Tian, Y.; Glenn, H. L.; Meldrum, D. R. K+ Regulates Ca2+ to Drive Inflammasome Signaling: Dynamic Visualization of Ion Flux in Live Cells. Cell Death Dis. 2015, 6 (10), 1-11. https://doi.org/10.1038/cddis.2015.277.
(76) Qiu, Y.; Huang, Y.; Chen, M.; Yang, Y.; Li, X.; Zhang, W. Mitochondrial DNA in NLRP3 Inflammasome Activation. Int. Immunopharmacol. 2022, 108.
(77) Heid, M. E.; Keyel, P. A.; Kamga, C.; Shiva, S.; Watkins, S. C.; Salter, R. D. Mitochondrial Reactive Oxygen Species Induces NLRP3-Dependent Lysosomal Damage and Inflammasome Activation. J. Immunol. 2013, 191 (10), 5230-5238. https://doi.org/10.4049/jimmunol.1301490. 42
(78) Kumrungsee, T.; Peipei Zhang; Yanaka, N.; Suda, T.; Kato, N. Emerging Cardioprotective Mechanisms of Vitamin B6: A Narrative Review. Eur. J. Nutr. 2022, 61 (2), 605-613. https://doi.org/10.1007/s00394-021-02665-2.
(79) Teng, Y.; Xu, D.; Yang, X.; Tang, H.; Tao, X.; Fan, Y.; Ding, Y. The Emerging Roles of Pyroptosis, Necroptosis, and Ferroptosis in Non-Malignant Dermatoses: A Review. J. Inflamm. Res. 2023, 16 (May), 1967-1977. https://doi.org/10.2147/JIR.S409699.
(80) Mikhaylov, D.; Hashim, P.; Nektalova, T.; Goldenberg, G. Systemic Psoriasis Therapies and Comorbid Disease in Patients with Psoriasis: A Review of Potential Risks and Benefits. J Clin Aesthet Dermatol 2019, 12 (6), 46-54.
(81) Jiao, Y.; Nan, J.; Mu, B.; Zhang, Y.; Zhou, N.; Yang, S.; Zhang, S.; Lin, W.; Wang, F.; Xia, A.; Cao, Z.; Chen, P.; Pan, Z.; Lin, G.; Pan, S.; Bin, H.; Li, L.; Yang, S. Discovery of a Novel and Potent Inhibitor with Differential Species-Specific Effects against NLRP3 and AIM2 Inflammasome-Dependent Pyroptosis. Eur. J. Med. Chem. 2022, 232. https://doi.org/10.1016/j.cjmech.2022.114194.
(82) Liu, T.; Aman, A.; Ainiwaer, M.; Ding, L.; Zhang, F.; Hu, Q.; Song, Y.; Ni, Y.; Tang, X. Evaluation of the Anti-Biofilm Effect of Poloxamer-Based Thermoreversible Gel of Silver Nanoparticles as a Potential Medication for Root Canal Therapy. Sci. Rep. 2021, 11 (1), 1-16. https://doi.org/10.1038/s41598-021-92081-7.
(83) Cook, M. T.; Haddow, P.; Kirton, S. B.; McAuley, W. J. Polymers Exhibiting Lower Critical Solution Temperatures as a Route to Thermoreversible Gelators for Healthcare. Adv. Funct. Mater. 2021, 31 (8). https://doi.org/10.1002/adfm.202008123.
(84) Fakhari, A.; Corcoran, M.; Schwarz, A. Thermogelling Properties of Purified Poloxamer 407. Heliyon 2017, 3 (8), e00390. https://doi.org/10.1016/j.heliyon.2017.c00390.
(85) ter Horst, B.; Moakes, R. J. A.; Chouhan, G.; Williams, R. L.; Moiemen, N. S.; Grover, L. M. A Gellan-Based Fluid Gel Carrier to Enhance Topical Spray Delivery. Acta Biomater. 2019, 89, 166-179. https://doi.org/10.1016/j.actbio.2019.03.036.
(86) Chang, Y.; Chu, W. L.; Chen, W. Y.; Zheng, J.; Liu, L.; Ruaan, R. C.; Higuchi, A. A Systematic SPR Study of Human Plasma Protein Adsorption Behavior on the Controlled Surface Packing of Self-Assembled Poly (Ethylene Oxide) Triblock 43 Copolymer Surfaces. J. Biomed. Mater. Res.-Part A 2010, 93 (1), 400-408. https://doi.org/10.1002/jbm.a.32558.
(87) Binder, L.; Mazál, J.; Petz, R.; Klang, V.; Valenta, C. The Role of Viscosity on Skin Penetration from Cellulose Ether-Based Hydrogels. Ski. Res. Technol. 2019, 25 (5), 725-734. https://doi.org/10.1111/srt.12709.
(88) Liao, W.; Aranson, I. S. Viscoelasticity Enhances Collective Motion of Bacteria. PNAS Nexus 2023, 2 (9), 1-10. https://doi.org/10.1093/pnasnexus/pgad291.
(89) Desmet, E.; Ramadhas, A.; Lambert, J.; Van Gele, M. In vitro Psoriasis Models with Focus on Reconstructed Skin Models as Promising Tools in Psoriasis Research. Exp. Biol. Med. 2017, 242 (11), 1158-1169. https://doi.org/10.1177/1535370217710637.
(90) Bochénska, K.; Smolińska, E.; Moskot, M.; Jakóbkiewicz-Banecka, J.; Gabig-Cimińska, M. Models in the Research Process of Psoriasis. Int. J. Mol. Sci. 2017, 18 (12), 1-17. https://doi.org/10.3390/ijms18122514.
(91) Neil, J. E.; Brown, M. B.; Williams, A. C. Human Skin Explant Model for the Investigation of Topical Therapeutics. Sci. Rep. 2020, 10 (1), 1-8. https://doi.org/10.1038/s41598-020-78292-4.
(92) Ahn, M.; Cho, W. W.; Park, W.; Lee, J. S.; Choi, M. J.; Gao, Q.; Gao, G.; Cho, D. W.; Kim, B. S. 3D Biofabrication of Diseased Human Skin Models in vitro. Biomater. Res. 2023, 27 (1), 1-13. https://doi.org/10.1186/s40824-023-00415-5.
(93) Schön, M. P.; Manzke, V.; Erpenbeck, L. Animal Models of Psoriasis Highlights and Drawbacks. J. Allergy Clin. Immunol. 2021, 147 (2), 439-455. https://doi.org/10.1016/j.jaci.2020.04.034.
(94) Garzorz-Stark, N.; Lauffer, F.; Krause, L.; Thomas, J.; Atenhan, A.; Franz, R.; Roenneberg, S.; Boehner, A.; Jargosch, M.; Batra, R.; Mueller, N. S.; Haak, S.; Groß, C.; Groß, O.; Traidl-Hoffmann, C.; Theis, F. J.; Schmidt-Weber, C. B.; Biedermann, T.; Eyerich, S.; Eyerich, K. Toll-like Receptor 7/8 Agonists Stimulate Plasmacytoid Dendritic Cells to Initiate TH17-Deviated Acute Contact Dermatitis in Human Subjects. J. Allergy Clin. Immunol. 2018, 141 (4), 1320-1333.e11. https://doi.org/10.1016/j.jaci.2017.07.045.
(95) Elias, M.; Zhao, S.; Le, H. T.; Wang, J.; Neurath, M. F.; Neufert, C.; Fiocchi, C.; 44 Rieder, F. IL-36 in Chronic Inflammation and Fibrosis-Bridging the Gap? J. Clin. Invest. 2021, 131 (2). https://doi.org/10.1172/JCI144336.
(96) Gangwar, R. S.; Gudjonsson, J. E.; Ward, N. L. Mouse Models of Psoriasis: A Comprehensive Review. J. Invest. Dermatol. 2022, 142 (3), 884-897. https://doi.org/10.1016/j.jid.2021.06.019.
(97) Bhalerao, J.; Bowcock, A. M. The Genetics of Psoriasis: A Complex Disorder of the Skin and Immune System. Hum. Mol. Genet. 1998, 7 (10), 1537-1545. https://doi.org/10.1093/hmg/7.10.1537.
(98) Bromley, S. K.; Larson, R. P.; Ziegler, S. F.; Luster, A. D. IL-23 Induces Atopic Dermatitis-Like Inflammation Instead of Psoriasis-Like Inflammation in CCR2-Deficient Mice. PLOS One 2013, 8 (3), 1-10. https://doi.org/10.1371/journal.pone.0058196.
(99) Swindell, W. R.; Michaels, K. A.; Sutter, A. J.; Diaconu, D.; Fritz, Y.; Xing, X.; Sarkar, M. K.; Liang, Y.; Tsoi, A.; Gudjonsson, J. E.; Ward, N. L. Imiquimod Has Strain-Dependent Effects in Mice and Does Not Uniquely Model Human Psoriasis. Genome Med. 2017, 9 (1), 1-21. https://doi.org/10.1186/s13073-017-0415-3.
(100) Jiang, W.; Zhu, F. G.; Bhagat, L.; Yu, D.; Tang, J. X.; Kandimalla, E. R.; La Monica, N.; Agrawal, S. A Toll-like Receptor 7, 8, and 9 Antagonist Inhibits Th1 and Th17 Responses and Inflammasome Activation in a Model of IL-23-Induced Psoriasis. J. Invest. Dermatol. 2013, 133 (7), 1777-1784. https://doi.org/10.1038/jid.2013.57.
(101) Miller, L. S. Toll-Like Receptors in Skin. Adv. Dermatol. 2008, 24 (C), 71-87. https://doi.org/10.1016/j.yadr.2008.09.004.
(102) von Stebut, E.; Boehncke, W. H.; Ghoreschi, K.; Gori, T.; Kaya, Z.; Thaci, D.; Schäffler, A. IL-17A in Psoriasis and Beyond: Cardiovascular and Metabolic Implications. Front. Immunol. 2020, 10 (January), 1-15. https://doi.org/10.3389/fimmu.2019.03096.
(103) Brembilla, N. C.; Senra, L.; Boehncke, W. H. The IL-17 Family of Cytokines in Psoriasis: IL-17A and Beyond. Front. Immunol. 2018, 9 (AUG). https://doi.org/10.3389/fimmu.2018.01682.
(104) Mosca, M.; Hong, J.; Hadeler, E.; Hakimi, M.; Liao, W.; Bhutani, T. The Role of IL-45 17 Cytokines in Psoriasis. ImmunoTargets Ther. 2021, 10 (August), 409-418. https://doi.org/10.2147/ITT.S240891.
(105) Branisteanu, D.; Cojocaru, C.; Diaconu, R.; Porumb, E.; Alexa, A.; Nicolescu, A.; Brihan, I.; Bogdanici, C.; Branisteanu, G.; Dimitriu, A.; Zemba, M.; Anton, N.; Toader, M.; Grechin, A.; Branisteanu, D. Update on the Etiopathogenesis of Psoriasis (Review). Exp. Ther. Med. 2022, 23 (3), 1-13. https://doi.org/10.3892/etm.2022.11124.
(106) Chan, J. R.; Blumenschein, W.; Murphy, E.; Diveu, C.; Wiekowski, M.; Abbondanzo, S.; Lucian, L.; Geissler, R.; Brodie, S.; Kimball, A. B.; Gorman, D. M.; Smith, K.; Malefyt, R. D. W.; Kastelein, R. A.; McClanahan, T. K.; Bowman, E. P. IL-23 Stimulates Epidermal Hyperplasia via TNF and IL-20R2-Dependent Mechanisms with Implications for Psoriasis Pathogenesis. J. Exp. Med. 2006, 203 (12), 2577-2587. https://doi.org/10.1084/jem.20060244.
(107) De, R.; Mignogna, C. The Histopathology of Psoriasis. Reumatismo 2007, 59 (SUPPL. 1), 46-48. https://doi.org/10.4081/reumatismo.2007.1s.46.
(108) Lo, Y. H.; Li, C. S.; Chen, H. L.; Chiang, C. Y.; Huang, C. C.; Tu, T. J.; Lo, T. H.; Choy, D. F.; Arron, J. R.; Chen, H. Y.; Liu, F. T. Galectin-8 Is Upregulated in Keratinocytes by IL-17A and Promotes Proliferation by Regulating Mitosis in Psoriasis. J. Invest. Dermatol. 2021, 141 (3), 503-511.e9. https://doi.org/10.1016/j.jid.2020.07.021.
(109) Yazici, A. C.; Tursen, U.; Apa, D. D.; Ikizoglu, G.; Api, H.; Baz, K.; Tasdelen, B. The Changes in Expression of ICAM-3, Ki-67, PCNA, and CD31 in Psoriatic Lesions before and after Methotrexate Treatment. Arch. Dermatol. Res. 2005, 297 (6), 249-255. https://doi.org/10.1007/s00403-005-0602-8.
(110) Lee, H. J.; Kim, M. Challenges and Future Trends in the Treatment of Psoriasis. Int. J. Mol. Sci. 2023, 24 (17). https://doi.org/10.3390/ijms241713313.
(111) Woo, Y. R.; Park, C. J.; Kang, H.; Kim, J. E. The Risk of Systemic Diseases in Those with Psoriasis and Psoriatic Arthritis: From Mechanisms to Clinic. Int. J. Mol. Sci. 2020, 21 (19), 1-27. https://doi.org/10.3390/ijms21197041.
(112) Thatiparthi, A.; Martin, A.; Liu, J.; Wu, J. Risk of Skin Cancer with Phototherapy in 46 Moderate-to-Severe Psoriasis. J. Clin. Aesthet. Dermatol. 2022, 15 (6), 68-75.
(113) Elmets, C. A.; Korman, N. J.; Prater, E. F.; Wong, E. B.; Rupani, R. N.; Kivelevitch, D.; Armstrong, A. W.; Connor, C.; Cordoro, K. M.; Davis, D. M. R.; Elewski, B. E.; Gelfand, J. M.; Gordon, K. B.; Gottlieb, A. B.; Kaplan, D. H.; Kavanaugh, A.; Kiselica, M.; Kroshinsky, D.; Lebwohl, M.; Leonardi, C. L.; Lichten, J.; Lim, H. W.; Mehta, N. N.; Paller, A. S.; Parra, S. L.; Pathy, A. L.; Siegel, M.; Stoff, B.; Strober, B.; Wu, J. J.; Hariharan, V.; Menter, A. Joint AAD-NPF Guidelines of Care for the Management and Treatment of Psoriasis with Topical Therapy and Alternative Medicine Modalities for Psoriasis Severity Measures. J. Am. Acad. Dermatol. 2021, 84 (2), 432-470. https://doi.org/10.1016/j.jaad.2020.07.087.
(114) Coondoo, A.; Phiske, M.; Verma, S.; Lahiri, K. Side-Effects of Topical Steroids: A Long Overdue Revisit. Indian Dermatol. Online J. 2014, 5 (4), 416. https://doi.org/10.4103/2229-5178.142483.
(115) Stacey, S. K.; McEleney, M. Topical Corticosteroids: Choice and Application. Am. Fam. Physician 2021, 103 (6), 337-343.
(116) Hwang, J.; Lio, P. A. Topical Corticosteroid Withdrawal (‘Steroid Addiction’): An Update of a Systematic Review. J. Dermatolog. Treat. 2022, 33 (3), 1293-1298. https://doi.org/10.1080/09546634.2021.1882659.
(117) Barrea, L.; Savanelli, M. C.; Di Somma, C.; Napolitano, M.; Megna, M.; Colao, A.; Savastano, S. Vitamin D and Its Role in Psoriasis: An Overview of the Dermatologist and Nutritionist. Rev. Endocr. Metab. Disord. 2017, 18 (2), 195-205. https://doi.org/10.1007/s11154-017-9411-6.
(118) Nicholas Brownstone, M. Psoriasis Therapies in 2024 and Beyond.
Dermatology Times. https://www.dermatologytimes.com/view/psoriasis-therapies-in-2024- and-beyond (accessed 2024 Apr. 7).
(119) Guo, J.; Zhang, H.; Lin, W.; Lu, L.; Su, J.; Chen, X. Signaling Pathways and Targeted Therapies for Psoriasis. Signal Transduct. Target. Ther. 2023, 8 (1).

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A sprayable polymeric scaffold composition for localized anti-inflammatory therapy comprising
a plurality of lipid nanorods, each nanorod comprising one or more lipid nanoparticles encapsulating an inhibitor selected from the group consisting of NLRP3 inflammasome inhibitors, AIM2 inflammasome inhibitors, and combinations thereof;
a thermogelling agent; and
a thickening agent;
wherein the composition is in a fluid sol state at room temperature and transitions to a semi-solid gel state at body temperature upon topical spraying on skin.

2. The composition of claim 1, wherein the nanoparticles comprises a vitamin B6 or a derivative thereof and a pegylated phospholipid.

3. The composition of claim 2, wherein the vitamin B6 or derivative thereof comprises pyridoxine dipalmitate, pyridoxine hydrochloride, pyridoxal. pyridoxal phosphate or combination thereof.

4. The composition of claim 2, wherein the pegylated phospholipid comprises DSPE PEG (2000) (carboxylic acid), PEG 1000, PEG 3400, mPEG-DSPE, DPPE PEG, PEGylated cholesterol or combination thereof.

5. The composition of claim 1, wherein lipid nanoparticles comprise pyridoxine dipalmitate and DSPE-PEG (2000) carboxylic acid.

6. The composition of claim 1, wherein the thermogelling agent comprises one or more of methylcellulose, hydroxypropyl methylcellulose (HPMC), chitosan and its derivatives, poly(N-isopropylacrylamide) (PNIPAM) and its copolymers, poly(ethylene oxide)-poly (propylene oxide)-poly(ethylene oxide) (PEO-PPO-PEO) block copolymers, xyloglucan, gelatin-based systems, poly(organophosphazenes), poly(ethylene glycol)-poly (lactic acid-co-glycolic acid)-poly(ethylene glycol) (PEG-PLGA-PEG) triblock copolymers, alginate-based systems, poloxamer 188, or poloxamer 407.

7. The composition of claim 1, wherein the thickening agent comprises one or more of methylcellulose, hydroxypropyl methylcellulose (HPMC), carboxymethylcellulose (CMC), microcrystalline cellulose, xanthan gum, guar gum, acacia gum (gum arabic), tragacanth gum, carbomers, polyvinylpyrrolidone (PVP), polyethylene glycol (PEG), pregelatinized starch, modified starch, sodium alginate, propylene glycol alginate, pectin, gelatin, colloidal silicon dioxide, bentonite clay, carrageenan, or mucin.

8. The composition of claim 1, wherein the inflammasome inhibitors comprise one or more of MCC950 (CRID3), parthenolide, bay 11-7082, glyburide, tranilast, CY-09, NT-0796, IC100, obovatol, OLT1177 (dapansutrile), VX-765 (belnacasan), oridonin, reversetrol, colchicine, auranofin, INF39, Fc11a-2, β-Hydroxybutyrate (BHB), NLRP3/AIM2-IN-3 (NA3), NLRP3 Inflammasome Inhibitor I, BAL-0028, or disulfuram.

9. The composition of claim 1, wherein the inhibitor encapsulated within the lipid nanorods is NLRP3/AIM2-IN-3.

10. The composition of claim 1, wherein the composition is packaged in a spraying applicator.

11. A method for treating an inflammatory skin disorder in a subject in need thereof, comprising topically administering to an affected area of the subject's skin a sprayable polymeric scaffold composition comprising
a plurality of lipid nanorods, each nanorod comprising one or more lipid nanoparticles encapsulating an inhibitor selected from the group consisting of NLRP3 inflammasome inhibitors, AIM2 inflammasome inhibitors, and combinations thereof;
a thermogelling agent; and
a thickening agent;
wherein the composition is in a fluid sol state at room temperature and transitions to a semi-solid gel state at body temperature upon application to the skin,
whereby a localized gel film is formed on the skin that provides sustained release of the encapsulated inhibitor, thereby attenuating inflammation in the affected area.

12. The method of claim 11, wherein the inflammatory skin disorder is selected from atopic dermatitis (eczema), psoriasis, contact dermatitis, rosace, seborrheic dermatitis, acne vulgaris, lupus erythematosus, lichen planus, cellulitis, hidradenitis suppurativa, dermatomyositis, scleroderma, urticaria (hives), vasculitis, or folliculitis.

13. The method of claim 11, wherein the nanoparticles comprises a vitamin B6 or a derivative thereof and a pegylated phospholipid.

14. The method of claim 13, wherein the vitamin B6 or derivative thereof comprises pyridoxine dipalmitate, pyridoxine hydrochloride, pyridoxal. pyridoxal phosphate or combination thereof.

15. The method of claim 13, wherein the pegylated phospholipid comprises DSPE PEG (2000) (carboxylic acid), PEG 1000, PEG 3400, mPEG-DSPE, DPPE PEG, PEGylated cholesterol or combination thereof.

16. The method of claim 11, wherein lipid nanoparticles comprise pyridoxine dipalmitate and DSPE-PEG (2000) carboxylic acid.

17. The method of claim 11, wherein the thermogelling agent comprises one or more of methylcellulose, hydroxypropyl methylcellulose (HPMC), chitosan and its derivatives, poly(N-isopropylacrylamide) (PNIPAM) and its copolymers, poly(ethylene oxide)-poly (propylene oxide)-poly(ethylene oxide) (PEO-PPO-PEO) block copolymers, xyloglucan, gelatin-based systems, poly(organophosphazenes), poly(ethylene glycol)-poly (lactic acid-co-glycolic acid)-poly(ethylene glycol) (PEG-PLGA-PEG) triblock copolymers, alginate-based systems, poloxamer 188, or poloxamer 407.

18. The method of claim 11, wherein the thickening agent comprises one or more of methylcellulose, hydroxypropyl methylcellulose (HPMC), carboxymethylcellulose (CMC), microcrystalline cellulose, xanthan gum, guar gum, acacia gum (gum arabic), tragacanth gum, carbomers, polyvinylpyrrolidone (PVP), polyethylene glycol (PEG), pregelatinized starch, modified starch, sodium alginate, propylene glycol alginate, pectin, gelatin, colloidal silicon dioxide, bentonite clay, carrageenan, or mucin.

19. The method of claim 11, wherein the inflammasome inhibitors comprises one or more of MCC950 (CRID3), parthenolide, bay 11-7082, glyburide, tranilast, CY-09, NT-0796, IC100, obovatol, OLT1177 (dapansutrile), VX-765 (belnacasan), oridonin, reversetrol, colchicine, auranofin, INF39, Fc11a-2, β-Hydroxybutyrate (BHB), NLRP3/AIM2-IN-3 (NA3), NLRP3 Inflammasome Inhibitor I, BAL-0028, or disulfuram.

20. The method of claim 11, wherein the inhibitor encapsulated within the lipid nanorods is NLRP3/AIM2-IN-3.
